(19) United States
(12) Patent Application Publication
(10) Pub. No.: US 2024/0335452 A1
(43) Pub. Date: Oct. 10, 2024

(54) COMPOSITIONS AND METHODS OF COMBINATION THERAPY FOR TARGETING LYMPHOMA

(71) Applicant: Cullgen (Shanghai), Inc., Shanghai (CN)

(72) Inventors: Jialiang WANG, Nashville, TN (US); Jing LIU, Oradell, NJ (US); Chengwei ZHANG, Shanghai (CN); Ting YANG, Shanghai (CN)

(21) Appl. No.: 18/294,717

(22) PCT Filed: Aug. 3, 2022
(86) PCT No.: PCT/CN2022/109992

§ 371 Date: Feb. 2, 2024

(30) Foreign Application Priority Data
Aug. 4, 2021 (WO) PCT/CN2021/110566

(51) Int. Cl. A61K 31/5377; A61K 31/4545; A61K 31/496; A61K 31/517; A61P 35/00

(52) U.S. Cl. CPC A61K 31/5377; A61K 31/4545; A61K 31/496; A61K 31/517; A61P 35/00

(57) ABSTRACT

Provided are compositions, their combinations, and their uses thereof, for example in treating a cancer, such as a lymphoma. Some embodiments include providing an Enhancer of Zeste Homolog 2 (EZH2) inhibitor and an immunomodulatory drug to a subject having the cancer.

### BACKGROUND

Lymphoma is a common form of blood cancer and may be the most common cancer of the hematopoietic system. The standard of care is chemotherapy and radiotherapy. Lymphomas may rise from lymphocytes or their progenitor cells and may affect any organ in the body or present a wide range of symptoms. According to data from National Cancer Institute, approximately 85,000 people are diagnosed with lymphoma annually and roughly 20,000 people are expected to die from this disease. Lymphoma may be divided into Hodgkin lymphoma (HL) and non-Hodgkin lymphoma (NHL). HL may account for approximately 10% of cases. HL may be characterized by the presence of Hodgkin and Reed-Sternberg (HRS) cells. Almost 90% of HL cases can be cured with a combination of surgery and intensive chemotherapy followed by stem cell transplantation. Other lymphomas may be categorized as non-Hodgkin lymphoma, which may include indolent follicular lymphoma (FL) or diffuse large B-cell lymphoma (DLBCL).

Some lymphomas have B cell origin. The germ center may support proliferation and survival during maturation of B cells. EZH2, a histone methyltransferase, may be upregulated in B cells in germ centers. EZH2 may include the catalytic subunit of the polycomb repressive complex 2 (PRC2). PRC2 may catalyze histone methylation, for example, a lysine at residue 27 of histone 3 (H3K27), leading to transcriptional silencing of surrounding chromatin. Transcriptional repressive activity of EZH2 may be involved in formation of germ centers and expansion and maturation of B cells through downregulation of genes involved in anti-proliferation, apoptosis, or lineage commitment.

Aberrant activation of EZH2 through mutations or overexpression may be found in lymphoma, particularly DLBCL, FL, or Burkitt's lymphoma. Somatic mutations of EZH2 at any of residues Y641, A677, or A687 may be frequent. These mutations may include gain-of-function mutations that increase levels of trimethylation at H3K27 (H3K27me3) in a cancer cell or malignant tissue. EZH2 activation may be associated with poor prognosis and therapeutic resistance. Because of this oncogenic role, small molecule inhibitors of EZH2 have been of interest for treating cancers such as lymphoma or other blood cancers.

Tazemetostat (EPZ-6438), a selective EZH2 methyltransferase inhibitor, has been approved for treating relapsed or refractory follicular lymphoma (FL) in adult patients who have received at least 2 prior systemic therapies and whose tumors are positive for an EZH2 mutation as detected by an FDA-approved test. Tazemetostat was reported to have a limited response rate and clinical benefit in patients with relapsed or refractory (R/R) DLBCL, irrespective of EZH2 mutational status. Additional signaling pathways may be required to compensate for a loss of EZH2 activity in some more aggressive blood cancers such as DLBCL.

Combination strategies have been recently described by Park et al., Combination Treatment with GSK126 and Pomalidomide Induces B-Cell Differentiation in EZH2 Gain-of-Function Mutant Diffuse Large B-Cell Lymphoma, Cancers 2020, 12:2541; Tong et al., Combined EZH2 Inhibition and IKAROS Degradation Leads to Enhanced Antitumor Activity in Diffuse Large B-cell Lymphoma, Clin. Cancer Res 2021, 27:5401-14; WO 2021/262962; and Carrancio et al., Abstract 3932: Pathway interaction and mechanistic synergy of CC-99282, a novel cereblon E3 ligase modulator (CELMoD) agent, with enhancer of zeste homolog 2 inhibitors (EZH2is), Cancer Res 2022, 82:(12_Supplement): 3932.

Despite recent advances, such as BTK inhibitors and CAR-T therapy, nearly a quarter of patients with lymphoma will eventually succumb to the disease due to primary and acquired resistance. Thus, there remains a need for innovative therapies.

### SUMMARY

The present invention demonstrates that combinations of EZH2 inhibitors and IMiDs are highly potent in lymphoma cell lines, including those that are refractory or resistant to either compound alone. The methods, compositions, and combinations provided herein may be useful to treat a range of blood cancers, including lymphomas in patients who are not responsive to either EZH2 inhibitors or IMiDs alone.

Provided herein are methods of treatment, comprising administering to a subject in need of a cancer treatment, a first compound comprising the structure of Formula I:

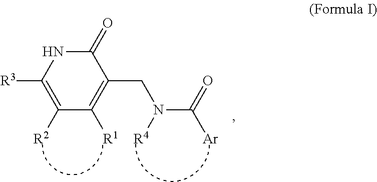

or a pharmaceutically acceptable salt, hydrate, solvate, prodrug, stereoisomer, tautomer, or analog thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
administering to the subject a second compound comprising an immunomodulatory drug.

The present disclosure also provides a composition for use in treating a subject in need of a cancer treatment, comprising: a first compound comprising the structure of Formula I:

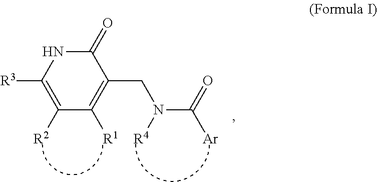

or a pharmaceutically acceptable salt, hydrate, solvate, prodrug, stereoisomer, tautomer, or analog thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and a second compound comprising an immunomodulatory drug.

The present disclosure also provides a method of treatment, comprising administering to a subject in need of a cancer treatment, a first compound comprising an EZH2 inhibitor; and administering to the subject a second compound comprising the structure of Formula II:

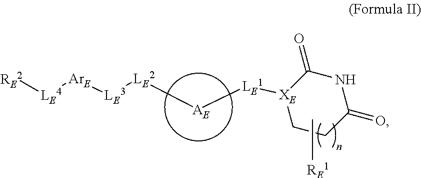

or a pharmaceutically acceptable salt, hydrate, solvate, prodrug, stereoisomer, tautomer, or analog thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl; R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

The present disclosure also provides a composition for use in treating a subject in need of a cancer treatment, comprising: a first compound comprising an EZH2 inhibitor; and a second compound comprising the structure of Formula II:

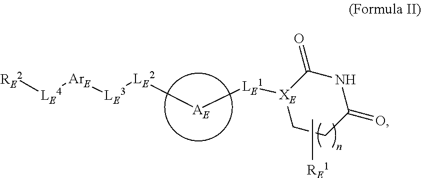

or a pharmaceutically acceptable salt, hydrate, solvate, prodrug, stereoisomer, tautomer, or analog thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}?- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring; Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

The present disclosure also provides a combination comprising: a first compound comprising an EZH2 inhibitor, and a second compound comprising an IMiD. In some embodiments of the combination, the EZH2 inhibitor comprises a structure of Formula I, as further described herein, or a pharmaceutically acceptable salt thereof. In some embodiments of the combination, the IMiD comprises a structure of Formula II, as further described herein, or a pharmaceutically acceptable salt thereof. In other embodiments of the combination, the EZH2 inhibitor comprises a structure of Formula I, or a pharmaceutically acceptable salt thereof, and the IMiD comprises a structure of Formula II, or a pharmaceutically acceptable salt thereof.

In some embodiments, the method, composition, or combination further comprises administering an additional cancer treatment to the subject. In some embodiments, the additional cancer treatment comprises chemotherapy, immunotherapy, radiation therapy, stem cell transplantation, targeted therapy. In some embodiments, the subject is a mammal. In some embodiments, the subject is a human.

The present disclosure also provides a method of treatment, comprising administering to a subject identified as having a relapsed or refractory cancer resistant to one or more EZH2 inhibitors or IMiDs, a first compound comprising an EZH2 inhibitor, and a second compound comprising an IMiD.

The present disclosure also provides a composition for use in treating a subject identified as having a relapsed or refractory cancer resistant to one or more EZH2 inhibitors or IMiDs, comprising a first compound comprising an EZH2 inhibitor, and a second compound comprising an IMiD.

The present disclosure also provides a combination for use in treating a subject identified as having a relapsed or refractory cancer resistant to one or more EZH2 inhibitors or IMiDs, comprising a first compound comprising an EZH2 inhibitor, and a second compound comprising an IMiD.

In some embodiments, the first compound is an Enhancer of Zeste Homolog 2 (EZH2) inhibitor. In some embodiments, the EZH2 inhibitor comprises a structure of Formula I, as further described herein, or a pharmaceutically acceptable salt thereof.

In some embodiments, the first compound is an EZH2 inhibitor comprising a structure of Formula I:

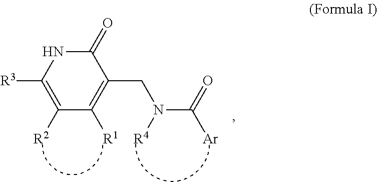

or a pharmaceutically acceptable salt, hydrate, solvate, prodrug, stereoisomer, tautomer, or analog thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.

In some embodiments, the first compound is an EZH2 inhibitor. In some embodiments, the EZH2 inhibitor is selected from the group consisting of EPZ-6438 (tazemetostat), CPI-1205 (lirametostat), C24 (CAS No. 1979157-17-9), CPI-0209 (CAS No. 2567686-02-4), CPI-1328 (CAS No. 2390367-27-6), PF-06821497 (CAS No. 1844849-10-1), DS-3201 (valemetostat), UNC1999 (CAS No. 1431612-23-5), SHR2254 (CAS No. 2098545-98-1), and GSK126 (CAS No. 1346574-57-9), or a pharmaceutically acceptable salt thereof. In some embodiments, the EZH2 inhibitor is EPZ-6438, CPI-1205, or C24. In some embodiments, the EZH2 inhibitor is GSK126. In some embodiments, the EZH2 inhibitor is CPI-0209 or SHR2554.

In some embodiments of the methods, compositions and combinations herein, the immunomodulatory drug is an immunomodulatory imide drug (IMiD). In some embodiments, the IMiD comprises a cereblon modulator (i.e., CRBN modulator). In some embodiments, the cereblon modulator is an IKZF1 and/or IZKF3 degrader or modulator. In some embodiments, the cereblon modulator is an IKZF1/3 degrader or modulator. In some embodiments, the IMiD is an IKZF1/3 degrader or modulator.

In some embodiments, the cereblon modulator comprises the structure of Formula II, as further described herein, or a pharmaceutically acceptable salt thereof.

In some embodiments, the second compound is a cereblon modulator comprising the structure of Formula II:

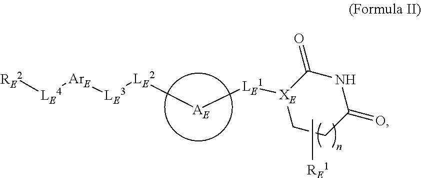

or a pharmaceutically acceptable salt, hydrate, solvate, prodrug, stereoisomer, tautomer, or analog thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}⁷ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of —R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

In some embodiments, the second compound is an IMiD, preferably a cereblon modulator. In some embodiments, the cereblon modulator is selected from the group consisting of CC-220 (iberdomide), CC-92480 (mezigdomide), CC-99282 (CAS No.: 2379572-34-4), DKY709 (CAS No.: 2291360-73-9), CFT7455 (CAS No.: 2504235-67-8), and CC-122 (avadomide), or a pharmaceutically acceptable salt thereof. In some embodiments, the second compound is CC-122, CC-220, or CC-92480. In some embodiments, the second compound is CC-99282 or CFT7455. In some embodiments, the second compound is thalidomide, lenalidomide, or pomalidomide.

In some embodiments, the first or second compound reduces cancer cell viability. In some embodiments, the first and second compound synergistically reduce cancer cell viability. In some embodiments, the cancer cell viability is reduced by at least 10%, at least 20%, or at least 50%. In some embodiments, the administration of the first and second compounds decreases viability of the cancer cells in the subject.

In some embodiments, the administration of the first and second compounds improves a cancer symptom in the subject, relative to a baseline cancer symptom. In some embodiments, the cancer symptom includes uncontrolled cell growth or division. In some embodiments, the cancer symptom includes unexplained pain, fever, night sweats, fatigue, unexplained weight loss, weakness, anorexia, or unexplained bleeding.

In some embodiments, the cancer is a blood cancer. In some embodiments, the blood cancer is a lymphoma, multiple myeloma, or leukemia. In some embodiments, the cancer is a lymphoma. In some embodiments, the lymphoma is a non-Hodgkin's lymphoma (NHL). In some embodiments, the non-Hodgkin's lymphoma (NHL) is follicular lymphoma (FL) or diffuse large B-cell lymphoma (DLBCL).

In some embodiments, the cancer is resistant to an EZH2 inhibitor or an IMiD. In some embodiments, the cancer in a blood cancer resistant to an EZH2 inhibitor or an IMiD. In some embodiments, the cancer in a lymphoma resistant to an EZH2 inhibitor or an IMiD.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A shows a graph of cell viability of SU-DHL-10 cells treated with EPZ-6438 in the presence or absence of 1 μM CC-122, 1 μM CC-220 or 1 nM CC-92480.

FIG. 1B shows a graph of cell viability of OCI-LY7 cells treated with EPZ-6438 in the presence or absence of 1 μM CC-122, 1 μM CC-220 or 1 nM CC-92480.

FIG. 1C shows a graph of cell viability of SU-DHL-10 cells treated with GSK126 in the presence or absence of 1 μM CC-122, 1 μM CC-220 or 1 nM CC-92480.

FIG. 1D shows a graph of cell viability of OCI-LY7 cells treated with GSK126 in the presence or absence of 1 μM CC-122, 1 μM CC-220 or 1 nM CC-92480.

FIG. 1E shows a graph of cell viability of SU-DHL-10 cells treated with CPI-1205 in the presence or absence of 1 μM CC-122, 1 μM CC-220 or 1 nM CC-92480.

FIG. 1F shows a graph of cell viability of OCI-LY7 cells treated with CPI-1205 in the presence or absence of 1 μM CC-122, 1 μM CC-220 or 1 nM CC-92480.

FIG. 2A shows a graph of cell viability of SU-DHL-10 cells treated with CC-122 in the presence or absence of a selection of 1 μM EPZ6438, C24, GSK126 or CPI-1205.

FIG. 2B shows a graph of cell viability of OCI-LY7 cells treated with CC-122 in the presence or absence of 1 μM EPZ6438, C24, GSK126 or CPI-1205.

FIG. 2C shows a graph of cell viability of SU-DHL-10 cells treated with CC-220 in the presence or absence of 1 μM EPZ6438, GSK126 or CPI-1205 or 2 μM C24.

FIG. 2D shows a graph of cell viability of OCI-LY7 cells treated with CC-220 in the presence or absence of 1 μM EPZ6438, C24, GSK126 or CPI-1205.

FIG. 3A shows a graph of cell viability of OCI-LY7 cells treated with EZH2 inhibitors CPI-0209 or SHR2554, or IMiDs CC-99282 or CFT7455.

FIG. 3B shows a graph of cell viability of SU-DHL-10 cells treated with EZH2 inhibitors CPI-0209 or SHR2554, or IMiDs CC-99282 or CFT7455.

FIG. 4A shows a graph of cell viability of OCI-LY7 cells treated with EZH2 inhibitor CPI-0209 in the presence or absence of 10 nM CC-99282 or 1 nM CFT7455.

FIG. 4B shows a graph of cell viability of OCI-LY7 cells treated with EZH2 inhibitor SHR2554 in the presence or absence of 10 nM CC-99282 or 1 nM CFT7455.

FIG. 4C shows a graph of cell viability of SU-DHL-10 cells treated with EZH2 inhibitor CPI-0209 in the presence or absence of 10 nM CC-99282 or 1 nM CFT7455.

FIG. 4D shows a graph of cell viability of SU-DHL-10 cells treated with EZH2 inhibitor SHR2554 in the presence or absence of 10 nM CC-99282 or 1 nM CFT7455.

FIG. 5A shows a graph of cell viability of OCI-LY7 cells treated with CC-99282 in the presence or absence of 100 nM CPI-0209 or SHR2554.

FIG. 5B shows a graph of cell viability of OCI-LY7 cells treated with CFT7455 in the presence or absence of 100 nM CPI-0209 or SHR2554.

FIG. 5C shows a graph of cell viability of SU-DHL-10 cells treated with CC-99282 in the presence or absence of 100 nM CPI-0209 or SHR2554.

FIG. 5D shows a graph of cell viability of SU-DHL-10 cells treated with CFT7455 in the presence or absence of 100 nM CPI-0209 or SHR2554.

### DETAILED DESCRIPTION

Disclosed herein are methods, compositions, and combinations used to treat a cancer in a subject in need thereof. The cancer may include but is not limited to lymphoma, a common form of blood cancer. Lymphoma commonly affects children or adolescents. Advances in targeted therapies have improved prognosis over the past two decades. However, nearly a quarter of patients with lymphoma will eventually die of the disease due to primary and acquired resistance. Enhancer of Zeste Homolog 2 (EZH2) may include a histone methyltransferase that plays a central role in regulation differentiation of B cell development. Aberrant activation of EZH2 impairs differentiation of B cells and promotes cellular transformation. Recurrent EZH2 gain-of-function mutations are found in a significant subset of follicular lymphoma (FL) and diffuse large B-cell lymphoma (DLBCL).

IKZF1 and IKZF3 are potential targets for immunomodulatory drugs (IMiDs). Thalidomide is an example of an IMiD. Thalidomide resulted in tens of thousands of birth defects in the 1950s. Despite these side effects, thalidomide or another IMiD may be useful for treating a blood cancer or immunological disease, e.g., multiple myeloma. Other IMiDs including but not limited to lenalidomide or pomalidomide may also be useful for treatment of multiple myeloma. Thalidomide or other IMiDs may kill myeloma cells through inducing degradation of IKZF1 and IKZF3. Some IMiDs may have improved IKZF1 or IKZF3 degradation efficiency. Some such improved IMiDs may include but is not limited to CC-122, CC-220, CC-92480 and CFT4755. For example, CC-92480 may be 1000-fold more potent than older IMiDs with regard to IKZF1 or IKZF3 degradation. In some cases, solid cancers are insensitive to IMiDs. Lenalidomide may be useful for the treatment of relapsed/refractory (R/R) mantle cell lymphoma as monotherapy, or also for R/R follicular lymphoma or marginal zone lymphoma in combination with rituximab. The use of lenalidomide in combination with tafasitamab, a humanized CD19 monoclonal antibody, may also be used to treat R/R DLBCL. In contrast, pomalidomide may sometimes have limited efficacy in lymphoma when administered alone. IMiDs such as CC-122 or CC-220 may be useful for treating lymphoma or myeloma.

The methods, compositions, and combinations provided herein may be useful to treat a range of blood cancers, including lymphomas in patients who are not responsive to either EZH2 inhibitors or IMiDs alone.

The combination of EZH2 inhibitors and IMiDs provided synergistic effects in EZH2-mutant and wild type cell lines. Co-treatment was significantly more potent than either class of agents alone, suggesting such combinations may be useful for the treatment of cancers, such as lymphoma, irrespective of EZH2 mutational status. In particular, the combination of an EZH2 inhibitor and an IMiD lowered the viability of cells comprising B cell lymphoma cells, suggesting such combinations may be useful in treating cancers, such as lymphoma or another blood cancer. Combinations of EZH2 inhibitors and IMiDs were found to sensitize the cancer cells to treatment and improved the therapeutic effects of either compound alone. Thus, such combinations may provide a useful treatment option for cancers that are refractory or resistant to treatment, such as with a previous treatment with an EZH2 inhibitor or an IMiD alone.

Each of the embodiments described herein envisions within its scope pharmaceutically acceptable salts of the small molecule compounds described herein. Accordingly, the phrase “or a pharmaceutically acceptable salt thereof” is implicit in the description of all small molecule compounds described herein.

The invention described herein suitably may be practiced in the absence of any element(s) not specifically disclosed herein. Thus, for example, in each instance herein any of the terms “comprising,” “consisting essentially of,” and “consisting of” may be replaced with either of the other two terms.

### First Compound

Disclosed herein are methods, compositions, and combinations comprising a first compound. The first compound may be used in any of the methods described herein, such as in a method of treatment. The first compound may be included in a composition for use in a method of treatment. The first compound may be included in a combination for use in the treatment of cancer. In some embodiments, the first compound comprises an EZH2 inhibitor. In some embodiments, the EZH2 inhibitor is included in a method of treatment, combination, or in a composition with a second compound.

In some embodiments, the first compound comprises a compound of Formula I:

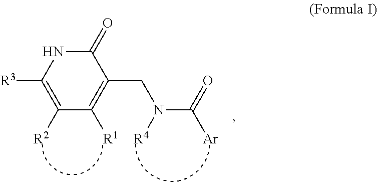

or a pharmaceutically acceptable salt, hydrate, solvate, prodrug, stereoisomer, tautomer, or analog thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.

In some embodiments, Ar is selected from optionally substituted mono-cyclic aryl. In some embodiments, Ar is selected from optionally substituted mono-cyclic heteroaryl. In some embodiments, Ar is selected from optionally substituted bi-cyclic aryl. In some embodiments, Ar is selected from optionally substituted bi-cyclic heteroaryl. In some embodiments, the first compound (e.g., an EZH2 inhibitor) is selected from the group consisting of a compound of Formulae Ia-1, Ia-2, Ia-3, Ia-4, Ia-5, lb-1, Ib-2, Ib-3, and Ib-4:

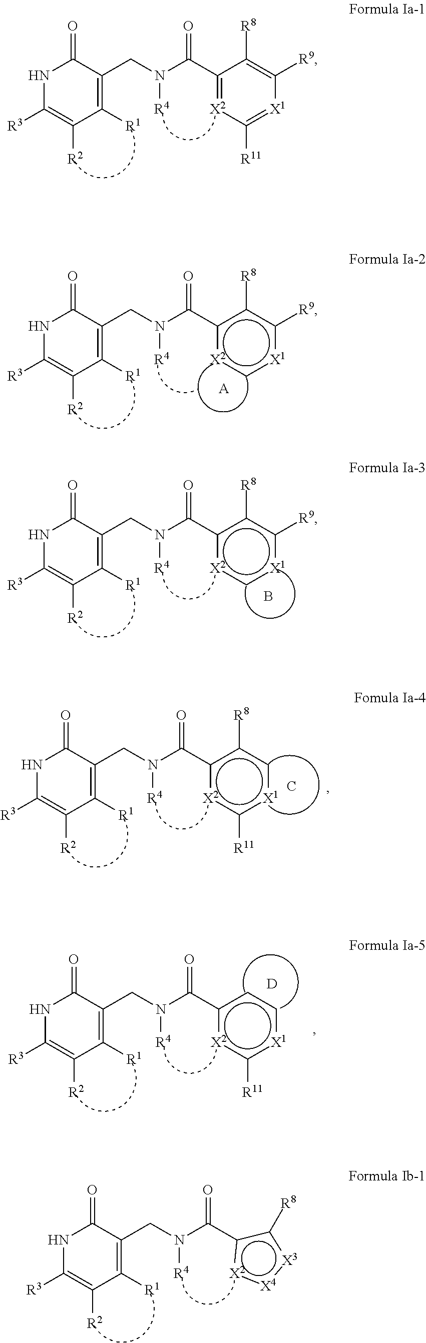

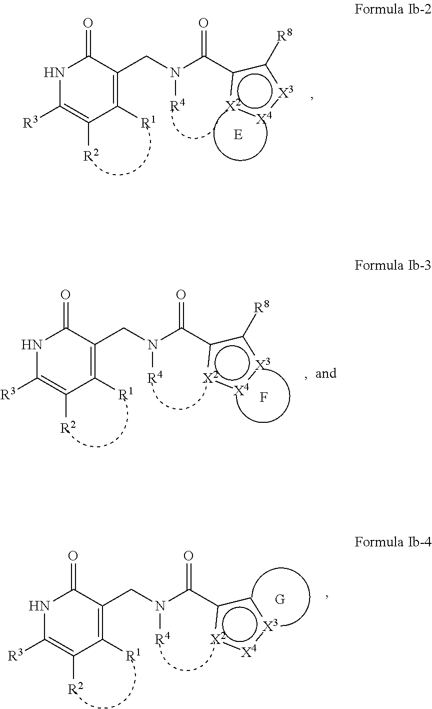

wherein R¹, R², R³, and R⁴, are defined in Formula I; Ring A, Ring B, Ring C, Ring D, Ring E, Ring F, and Ring G are independently selected from optionally substituted cycloalkyl, optionally substituted heterocyclyl, optionally substituted aryl, or optionally substituted heteroaryl; X¹ is selected from N or CR¹⁰; X² is selected from N, NR¹² or CR¹²; X³ is selected from S, N, NR⁹ or CR⁹; X⁴ is selected from S, N, NR¹¹ or CR¹¹; R, R¹⁰, and R¹², are independently selected from a bond, hydrogen, halogen, hydroxy, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ cycloalkyl, optionally substituted 3-12 membered heterocyclyl, optionally substituted C₁-C₈ alkoxy, optionally substituted C₃-C₁₂ cycloalkoxy, optionally substituted C₁-C₈ alkylamino, and optionally substituted C₃-C₁₂ cycloalkylamino; R⁹ is selected from groups consisting of -L^{9a}-R^{9a}-L^{9b}-R^{9b}, wherein L^{9a} and L^{9b} are independently selected from null, —O—, —S—, —C(O)—, —S(O)—, —S(O)₂—, —NR^{9′}—, —CR^{9′}R^{9″}—, —C(O)—NR^{9′}—, and —S(O)₂—NR^{9′}—; R^{9a} is selected from null, and optionally substituted divalent groups consisting of C₁-C₈ alkylene, C₂-C₈ alkenylene, C₂-C₈ alkynylene, C₁-C₈ heteroalkylene, C₂-C₈ heteroalkenylene, C₂-C₈ heteroalkynylene, C₃-C₁₂ membered cycloalkylene, 3-12 membered heterocyclylene, aryl, and heteroaryl; R^{9b} is selected from hydrogen, halogen, hydroxy, amino, cyano, nitro, and optionally substituted groups consisting of C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₃-C₁₂ membered cycloalkyl, 3-12 membered heterocyclyl, aryl, and heteroaryl; and R^{9′} and R^{9″}, at each occurrence, are independently selected from null, hydrogen, halogen, cyano, hydroxyl, amino, and optionally substituted groups consisting of C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, C₁-C₈ alkylamino, C₁-C₈ alkoxyC₁-C₈alkyl, C₁-C₈ haloalkyl, C₁-C₈ hydroxyalkyl, C₁-C₈alkylaminoC₁-C₈alkyl, C₃-C₁₂ cycloalkyl, C₃-C₁₂ cycloalkoxy, C₃-C₁₂ cycloalkylamino, 3-12 membered heterocyclyl, 3-12 membered heterocyclyloxy, 3-12 membered heterocyclylamino, arylalkyl, aryl-O—, aryl-amino-, heteroarylalkyl, heteroaryl-O—, heteroarylalkyl-amino-, aryl, and heteroaryl; or two of independent R^{9a}, R^{9b}, R^{9′} and R^{9″}, together with the atoms to which they are connected, can optionally form a C₃-C₁₂ membered cycloalkyl ring, or a 3-12 membered heterocyclyl ring; R¹¹ is selected from groups consisting of -L^{11a}-R^{11a}-L^{11b}R^{11b}, wherein L^{11a} and L^{11b} are independently selected from null, —O—, —S—, —C(O)—, —S(O)—, —S(O)₂—, —NR^{11′}—, —CR^{11′}R^{11″}—, —C(O)—NR^{11′}—, and —S(O)₂—NR^{11′}—; R^{11a} is selected from null, and optionally substituted divalent groups consisting of C₁-C₈ alkylene, C₂-C₈ alkenylene, C₂-C₈ alkynylene, C₁-C₈ heteroalkylene, C₂-C₈ heteroalkenylene, C₂-C₈ heteroalkynylene, C₃-C₁₂ membered cycloalkylene, 3-12 membered heterocyclylene, aryl, and heteroaryl; R^{11b} is selected from hydrogen, halogen, hydroxy, amino, cyano, nitro, and optionally substituted groups consisting of C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₃-C₁₂ membered cycloalkyl, 3-12 membered heterocyclyl, aryl, and heteroaryl; and R^{11b′} and R^{11″}, at each occurrence, are independently selected from null, hydrogen, halogen, cyano, hydroxyl, amino, and optionally substituted groups consisting of C₁-C₈ alkyl, C₂-C₈ alkenyl, C₂-C₈ alkynyl, C₁-C₈ heteroalkyl, C₂-C₈ heteroalkenyl, C₂-C₈ heteroalkynyl, C₁-C₈ alkoxy, C₁-C₈ alkylamino, C₁-C₈ alkoxyC₁-C₈ alkyl, C₁-C₈ haloalkyl, C₁-C₈ hydroxyalkyl, C₁-C₈alkylaminoC₁-C₈alkyl, C₃-C₁₂ cycloalkyl, C₃-C₁₂ cycloalkoxy, C₃-C₁₂ cycloalkylamino, 3-12 membered heterocyclyl, 3-12 membered heterocyclyloxy, 3-12 membered heterocyclylamino, arylalkyl, aryl-O—, aryl-amino-, heteroarylalkyl, heteroaryl-O—, heteroarylalkyl-amino-, aryl, and heteroaryl; or two of independent Ru^{11a}, R^{11b}, R^{11′} and R^{11″}, together with the atoms to which they are connected, can optionally form a C₃-C₁₂ membered cycloalkyl ring, or a 3-12 membered heterocyclyl ring; and R⁴ is selected from a bond, hydrogen, optionally substituted C₁-C₈ alkyl, optionally substituted C₁-C₈ heteroalkyl, optionally substituted 3-12 membered cycloalkyl, and optionally substituted 3-12 membered heterocyclyl, or R⁴ is optionally connected to Ar through X², R¹², Ring A, or Ring E, and together with the atoms to which they are connected, can optionally form optionally substituted 4-7 membered heterocyclyl.

In some embodiments, Ring A, Ring B, Ring D, Ring E are independently selected from optionally substituted aryl, or optionally substituted heteroaryl. Ring A is selected from optionally substituted aryl, or optionally substituted heteroaryl. Ring B is selected from optionally substituted aryl, or optionally substituted heteroaryl. In some embodiments, Ring C is selected from optionally substituted cycloalkyl, optionally substituted heterocyclyl. Ring D is selected from optionally substituted aryl, or optionally substituted heteroaryl. Ring E is selected from optionally substituted aryl, or optionally substituted heteroaryl. In some embodiments, X¹ is N. In some embodiments, X¹ is CR¹⁰. In some embodiments, X² is N. In some embodiments, X² is NR¹². In some embodiments, X² is CR¹². In some embodiments, X³ is S. In some embodiments, X³ is N. In some embodiments, X³ is NR⁹. In some embodiments, X³ is CR⁹. In some embodiments, X⁴ is S. In some embodiments, X⁴ is N. In some embodiments, X⁴ is NR¹¹. In some embodiments, X⁴ is CR¹¹. In some embodiments, R⁴ is selected from hydrogen, or optionally substituted C₁-C₈ alkyl. In some embodiments, R⁴ is H. In some embodiments, R⁴ and X², together with the atoms to which they are connected, can optionally form 4-7 membered heterocyclyl. In some embodiments, R⁴ and R¹², together with the atoms to which they are connected, can optionally form 4-7 membered heterocyclyl. In some embodiments, R⁴ is a bivalent group and connected to Ar group at the X² or R¹² position, and R⁴ is an optionally substituted C₁-C₈ alkylene group. In some embodiments, R⁴ is (CH₂)₂ or (CH₂)₃ and connected to Ar group at the X², and X² is C. In some embodiments, R⁴ is (CH₂)₂ or (CH₂)₃ and connected to Ar group at the R¹². In some embodiments, R⁴ is a bivalent group and connected to Ar group though Ring A or Ring E. In some embodiments, the first compound (e.g., EZH2 inhibitor) is selected from a compound of Formulae Ia-1a, Ia-1b, Ia-2a, Ia-2b, Ia-3a, Ia-3b, Ia-3c, Ia-3d, Ia-4a, Ia-4b, Ia-4c, Ia-4d, Ia-5a, Ia-5b, Ib-1a, Ib-1b, Ib-1c, Ib-1d, Ib-2a, or Ib-2b:

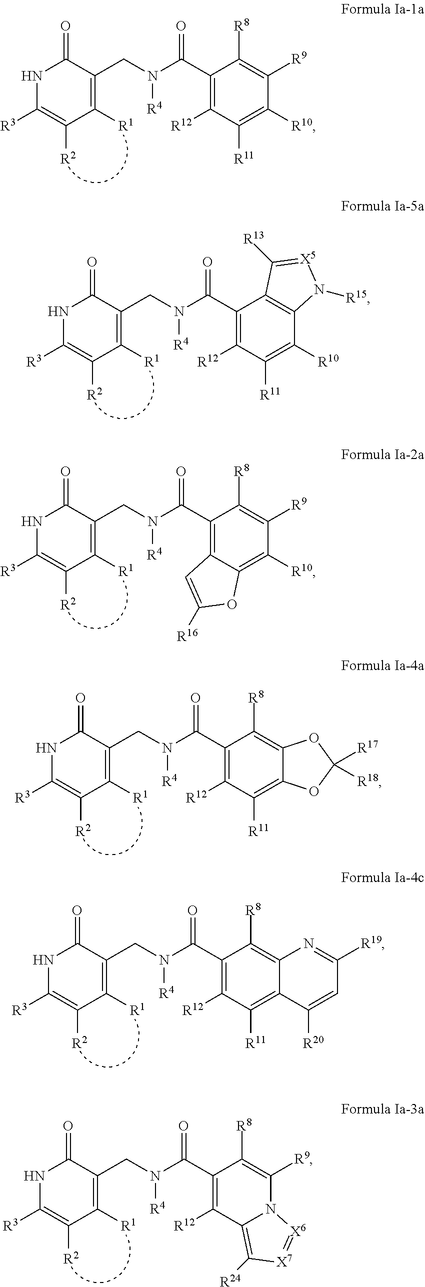

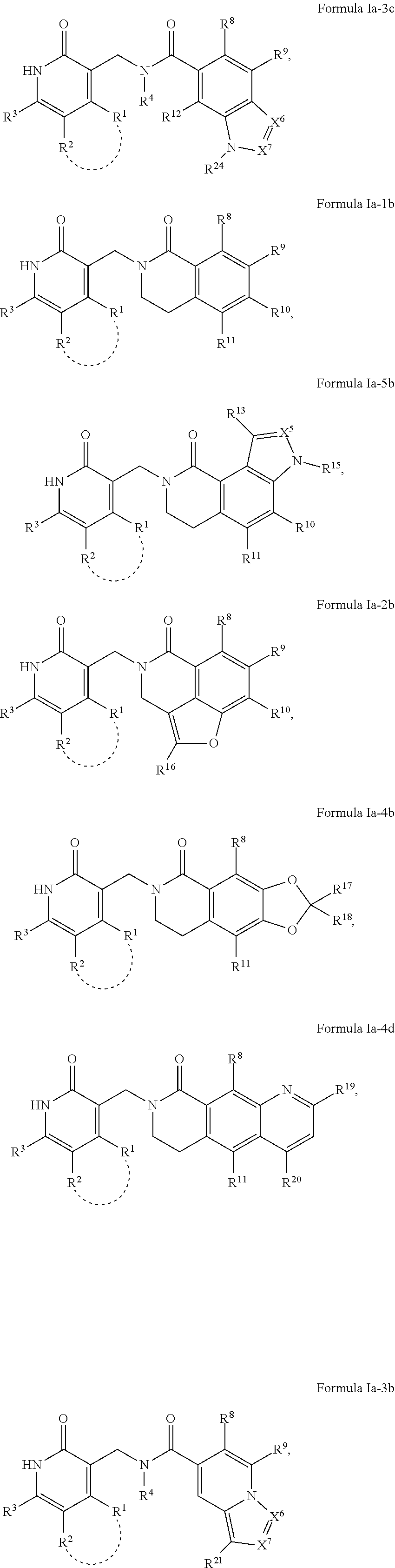

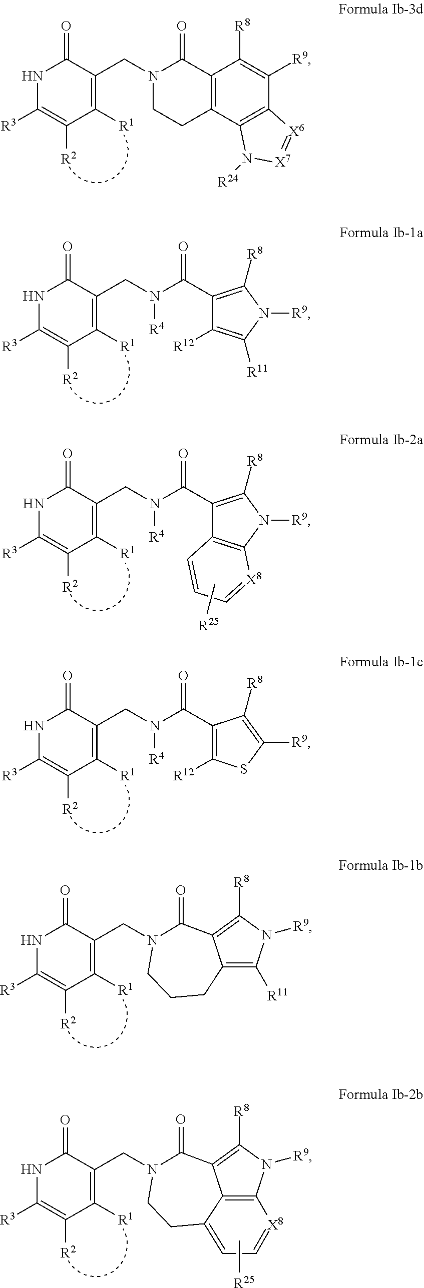

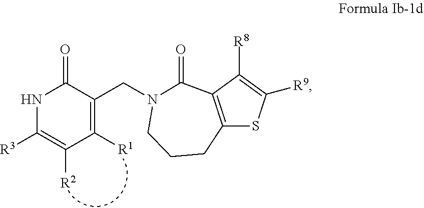

wherein R¹, R², R³, and R⁴ are defined in Formula I; R⁸, R⁹, R¹⁰, R¹¹, and R¹² are defined in Formulae Ia-1, Ia-2, Ia-3, Ia-4, Ia-5, lb-1, Ib-2, Ib-3, and Ib-4; X⁵ is selected from N or CR¹⁴; X⁶ is selected from N or CR²²; X⁷ is selected from N or CR²³; R¹³, R¹⁴, R¹⁹, R²¹, R²², R²³, and R²⁵ are independently selected from selected from hydrogen, halogen, hydroxy, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ cycloalkyl, optionally substituted C₃-C₁₂ heterocycloalkyl, optionally substituted 3-12 membered heterocyclyl, optionally substituted C₁-C₈ alkoxy, optionally substituted C₃-C₁₂ cycloalkoxy, optionally substituted C₁-C₈ alkylamino, optionally substituted C₃-C₁₂ cycloalkylamino, optionally substituted aryl, and optionally substituted heteroaryl;
R¹, R¹⁷, and R²⁴ are independently selected from selected from hydrogen, halogen, hydroxy, cyano, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ cycloalkyl, optionally substituted 3-12 membered heterocyclyl, optionally substituted aryl, and optionally substituted heteroaryl; R¹⁶ is selected from groups consisting of -L^{16b}-R^{16b}, wherein L^{16b} is selected from null, —O—, —S—, —C(O)—, —S(O)—, —S(O)₂—, —NR^{16′}—, —CR^{16′}R^{6″}—, —C(O)—NR^{16′}—, and —S(O)₂—NR^{16′}—; R^{16b} is selected from hydrogen, halogen, hydroxy, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ membered cycloalkyl, optionally substituted 3-12 membered heterocyclyl, optionally substituted aryl, and optionally substituted heteroaryl; and R^{16′} and R^{16″}, at each occurrence, are independently selected from null, hydrogen, halogen, cyano, hydroxyl, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted C₃-C₁₂ cycloalkoxy, optionally substituted C₃-C₁₂ cycloalkylamino, optionally substituted 3-12 membered heterocyclyl, optionally substituted 3-12 membered heterocyclyloxy, optionally substituted 3-12 membered heterocyclylamino, optionally substituted arylalkyl, optionally substituted aryl-O—, optionally substituted aryl-amino-, optionally substituted heteroarylalkyl, optionally substituted heteroaryl-O—, optionally substituted heteroarylalkyl-amino-, optionally substituted aryl, and optionally substituted heteroaryl; or two of each R^{16b}, R^{16′} and R^{16″}, together with the atoms to which they are connected, can optionally form an optionally substituted C₃-C₁₂ membered cycloalkyl ring, or an optionally substituted 3-12 membered heterocyclyl ring; R¹⁸ is selected from groups consisting of —R^{1a}-L^{18b}-R^{18b}, wherein L^{18b} is selected from null, —O—, —S—, —C(O)—, —S(O)—, —S(O)₂—, —NR^{18′}—, —CR^{18′}R^{18″}—, —C(O)—NR^{18′}—, and —S(O)₂—NR^{18′}—; R^{18a} is selected from null, and divalent groups consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl; R^{18b} is selected from hydrogen, halogen, hydroxy, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ membered cycloalkyl, optionally substituted 3-12 membered heterocyclyl, optionally substituted aryl, and optionally substituted heteroaryl; and R^{18′} and R^{18″}, at each occurrence, are independently selected from null, hydrogen, halogen, cyano, hydroxyl, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted C₃-C₁₂ cycloalkoxy, optionally substituted C₃-C₁₂ cycloalkylamino, optionally substituted 3-12 membered heterocyclyl, optionally substituted 3-12 membered heterocyclyloxy, optionally substituted 3-12 membered heterocyclylamino, optionally substituted arylalkyl, optionally substituted aryl-O—, optionally substituted aryl-amino-, optionally substituted heteroarylalkyl, optionally substituted heteroaryl-O—, optionally substituted heteroarylalkyl-amino-, optionally substituted aryl, and optionally substituted heteroaryl; or two of each R^{18a}, R^{18b}, R^{18′} and R^{18″}, together with the atoms to which they are connected, can optionally form an optionally substituted C₃-C₁₂ membered cycloalkyl ring, or an optionally substituted 3-12 membered heterocyclyl ring; R²⁰ is selected from groups consisting of -L^{20a}-R^{20a}-L^{20b}R^{20b}, wherein L^{20a} and L^{20b} are independently selected from null, —O—, —S—, —C(O)—, —S(O)—, —S(O)₂—, —NR^{20′}—, —CR^{20′}R^{20″}—, —C(O)—NR^{20′}—, and —S(O)₂—NR^{20′}—; R^{20a} is selected from null, optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl; R^{20b} is selected from hydrogen, halogen, hydroxy, amino, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₃-C₁₂ membered cycloalkyl, optionally substituted 3-12 membered heterocyclyl, optionally substituted aryl, and optionally substituted heteroaryl; R^{20′} and R^{20″}, at each occurrence, are independently selected from null, hydrogen, halogen, cyano, hydroxyl, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, C₁-C₈ alkoxy, optionally substituted C₁-C₈ alkylamino, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted C₃-C₁₂ cycloalkoxy, optionally substituted C₃-C₁₂ cycloalkylamino, optionally substituted 3-12 membered heterocyclyl, optionally substituted 3-12 membered heterocyclyloxy, optionally substituted 3-12 membered heterocyclylamino, optionally substituted arylalkyl, optionally substituted aryl-O—, optionally substituted aryl-amino-, optionally substituted heteroarylalkyl, optionally substituted heteroaryl-O—, optionally substituted heteroarylalkyl-amino-, optionally substituted aryl, and optionally substituted heteroaryl, or two of independent R^{20a}, R^{20b}, R^{20′} and R^{20″}, together with the atoms to which they are connected, can optionally form an optionally substituted C₃-C₁₂ membered cycloalkyl ring, or an optionally substituted 3-12 membered heterocyclyl ring.

In some embodiments, R¹ is selected from hydrogen, optionally substituted C₁-C₈ alkyl, optionally substituted C₁-C₈ alkoxy, and optionally substituted C₁-C₈ alkylthio. In some embodiments, R¹ is selected from H, CH₃, CH₂CH₃, CH₂CH₂CH₃, OCH₃, OCH₂CH₃, OCH(CH₃)₂, OCH₂CH₂CH₃, SCH₃, SCH₂CH₃, and SCH₂CH₂CH₃. In some embodiments, R¹ is selected from CH₃, CH₂CH₂CH₃, OCH₃, and SCH₃.

In some embodiments, R² is selected from hydrogen, halogen, and optionally substituted C₁-C₈ alkyl. In some embodiments, R² is selected from H, F, Cl, and CH₃. In some embodiments, R² is H.

In some embodiments, R¹ and R², together with the atom to which they are connected, form an optionally substituted 3-6 membered carbocyclyl, optionally substituted 3-6 membered heterocyclyl, 6 membered aryl, or optionally substituted 5-6 membered heteroaryl ring.

In some embodiments, R³ is selected from hydrogen, halogen, and optionally substituted C₁-C₈ alkyl. In some embodiments, R³ is selected from H, F, Cl, and CH₃. In some embodiments, R³ is CH₃.

In some embodiments, the optionally substituted 2-pyridone moiety,

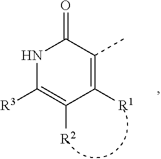

of the EZH2 inhibitor includes a moiety of Formula P-1, P-2, P-3, P-4, P-5, P-6, P-7, P-8, or P-9:

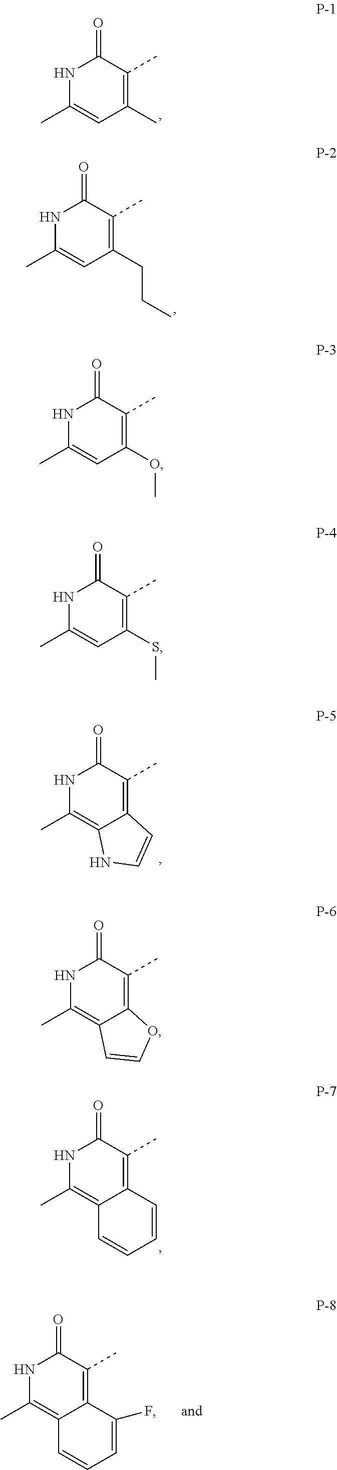

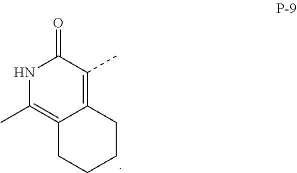

In some embodiments, X⁵ is N. In some embodiments, X⁵ is CR¹⁴. In some embodiments, X⁶ is N. In some embodiments, X⁶ is CR²². In some embodiments, X⁷ is N. In some embodiments, X⁷ is CR²³.

In some embodiments, R⁸ is selected from hydrogen, halogen, cyano, and optionally substituted C₁-C₈ alkyl. In some embodiments, R⁸ is selected from H, CH₃, CH₂CH₃, CN, F, Cl, and Br. In some embodiments, R⁸ is selected from CH₃, CH₂CH₃, and Cl.

In some embodiments, L^{9a} and L^{9b} are independently selected from null, —O—, —NR^{9′}—, —CR^{9′}R^{9″}—, —C(O)—, and —C(O)—NR^{9′}—. In some embodiments, L^{18b} is selected from null, —O—, —NR^{18′}—, —CR^{18′}R^{18″}—, —C(O)—, and —C(O)—NR^{18′}—. In some embodiments, R^{9′}, R^{9″}, R^{18′} and R^{18″} at each occurrence, are independently selected from null, hydrogen, halogen, optionally substituted C₁-C₈ alkyl, optionally substituted C₂-C₈ alkenyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, optionally substituted 3-12 membered heterocyclyl, optionally substituted 3-12 membered heterocyclyloxy, and optionally substituted 3-12 membered heterocyclylamino. In some embodiments, when R^{9″} is null, —CR^{9′}R^{9″}— is —C(═R^{9′})—. In some embodiments, when R^{9″} is null, —CR^{9′}R^{9″}—R^{9a}— is —C(R^{9′})═R^{9a}-. In some embodiments, when R^{9″} is null, —CR^{9′}R^{9″}—R^{9b} is —C(R^{9′})═R^{9b}. In some embodiments, R^{9a} and R^{18a} are independently selected from null, optionally substituted C₁-C₈ alkylene, C₂-C₈ alkynylene, optionally substituted C₃-C₁₂ membered cycloalkylene, optionally substituted 3-12 membered heterocyclylene, optionally substituted aryl, and optionally substituted heteroaryl. In some embodiments, R^{9a} and R^{18a} are independently selected from null, optionally substituted C₃-C₁₂ membered cycloalkylene, and optionally substituted 3-12 membered heterocyclylene.

In some embodiments, R^{9a} and R^{18a} are independently selected from null, or optionally substituted groups consisting of

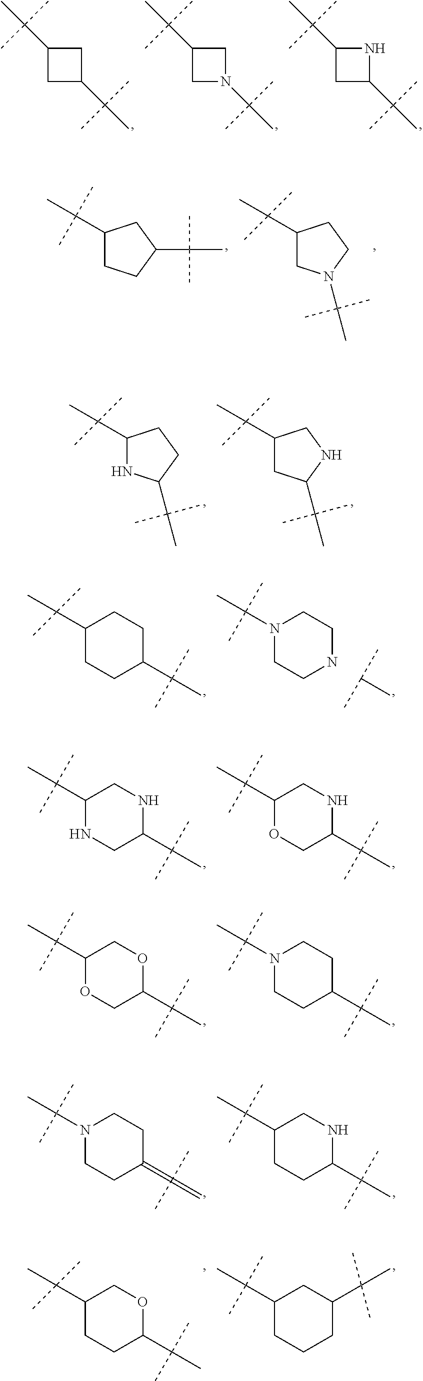

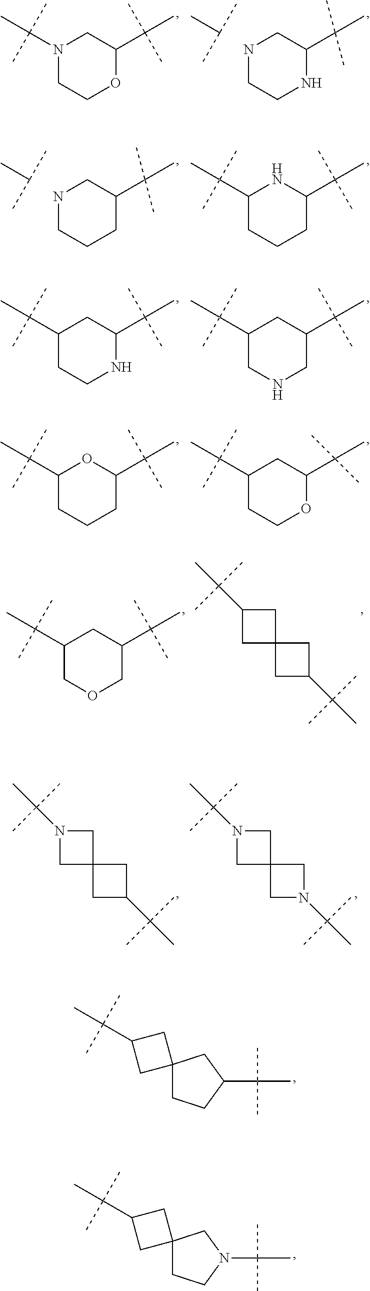

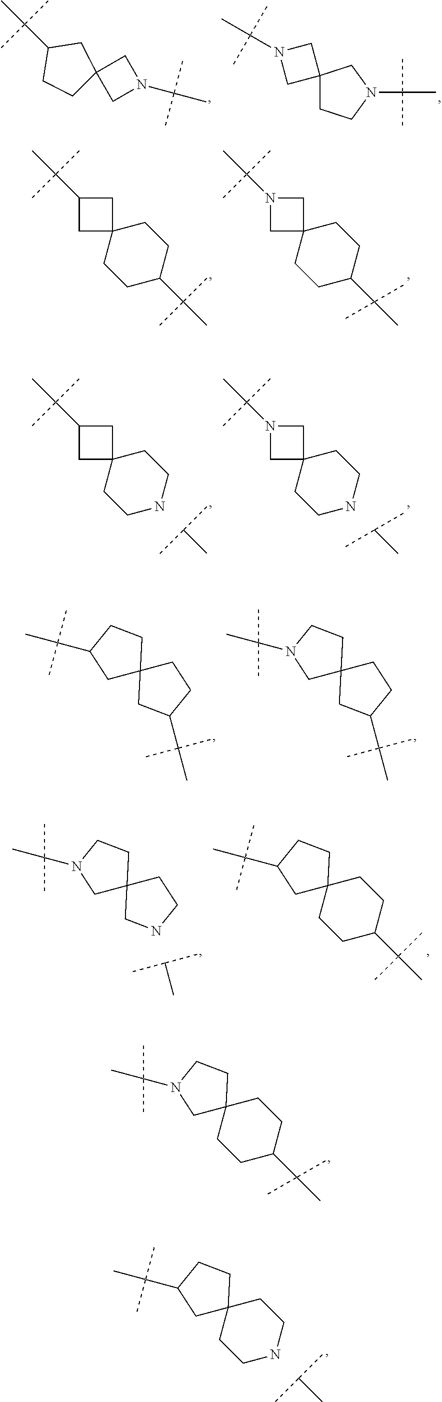

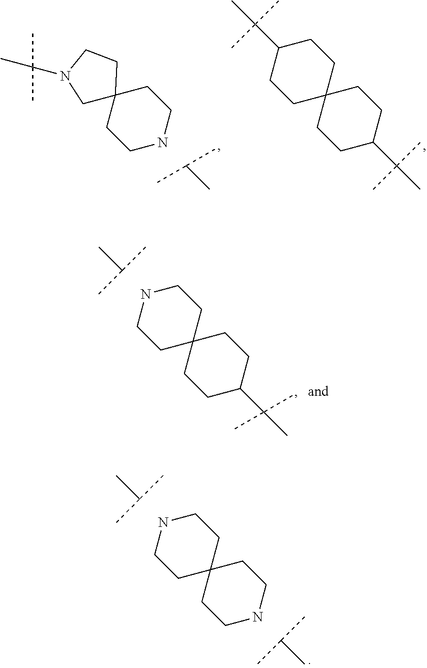

In some embodiments, R^{9a} and R^{18a} are independently selected from null,

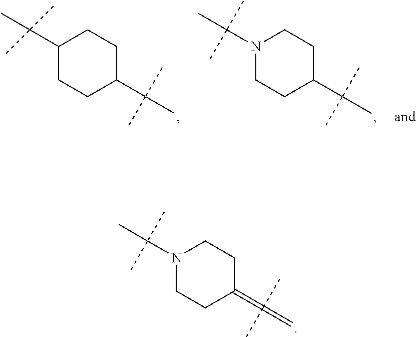

In some embodiments, R^{9b} and R^{18b} are independently selected from hydrogen, halogen, cyano, optionally substituted C₁-C₈ alkyl, optionally substituted C₂-C₈ alkenyl, optionally substituted C₂-C₈ alkynyl, optionally substituted C₃-C₁₂ membered cycloalkyl, optionally substituted 3-12 membered heterocyclyl, optionally substituted aryl, and optionally substituted heteroaryl. In some embodiments, R^{9b} and R^{18b} are independently selected from hydrogen, halogen, optionally substituted C₁-C₈ alkyl, optionally substituted C₂-C₈ alkenyl, optionally substituted C₂-C₈ alkynyl, and optionally substituted groups consisting of

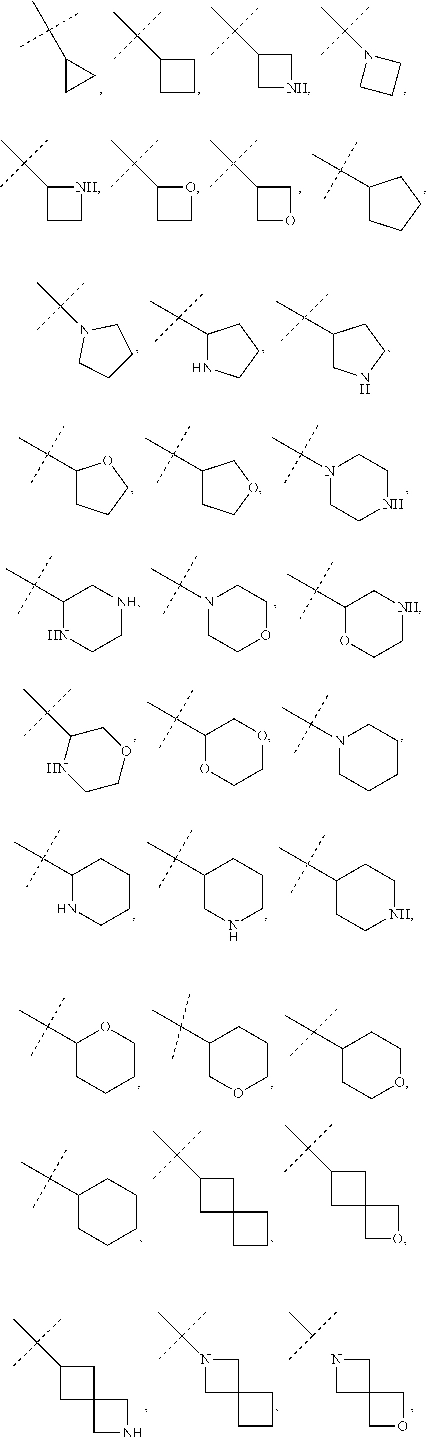

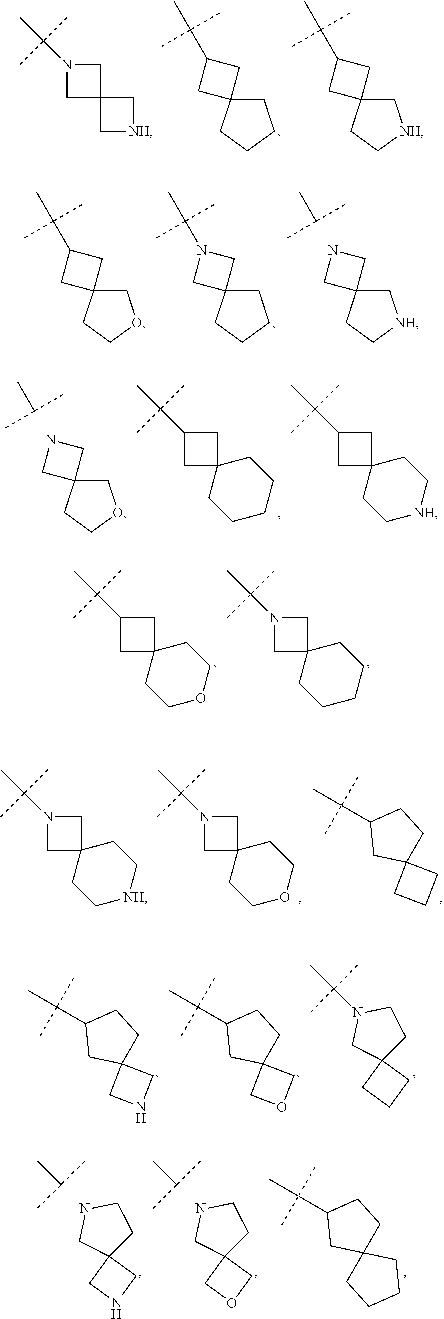

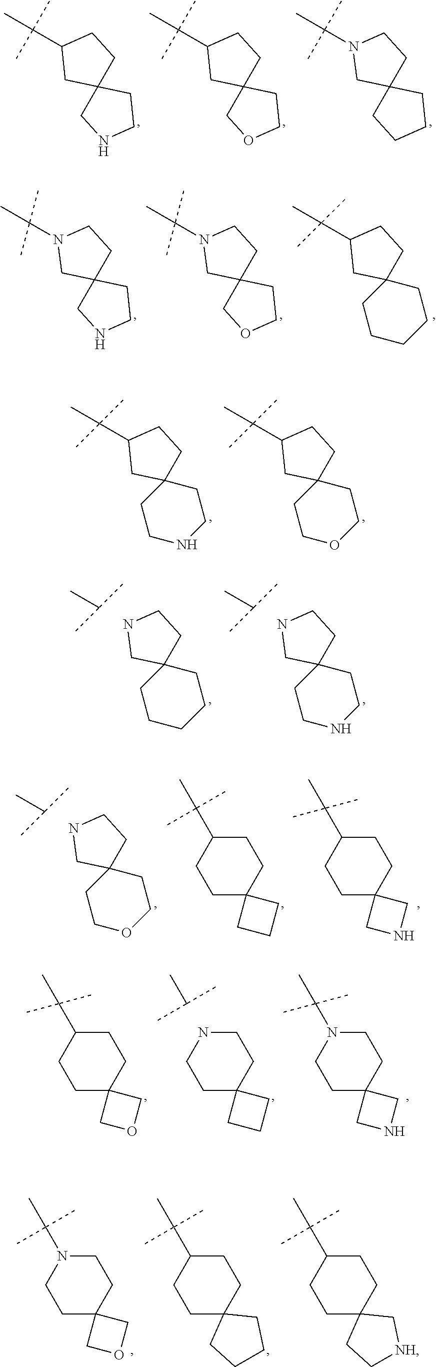

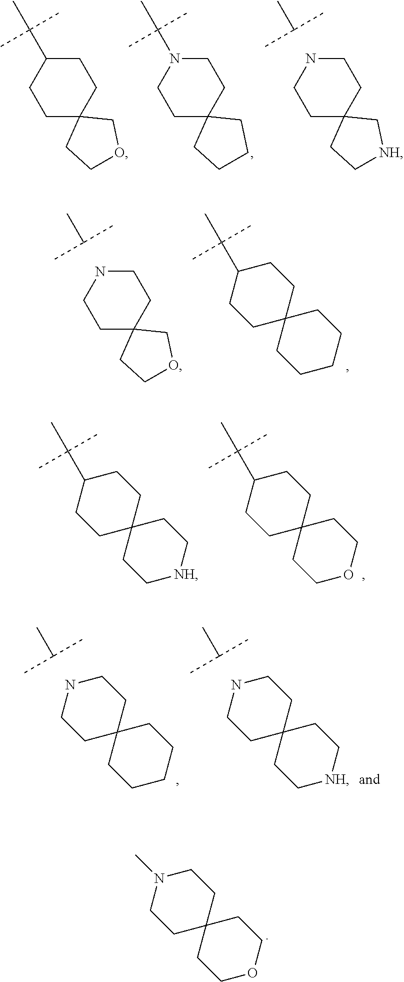

In some embodiments, R^{9b} and R^{18b} are independently selected from hydrogen, halogen, optionally substituted C₁-C₈ alkyl, optionally substituted C₂-C₈ alkenyl, optionally substituted C₂-C₈ alkynyl, and optionally substituted groups consisting of

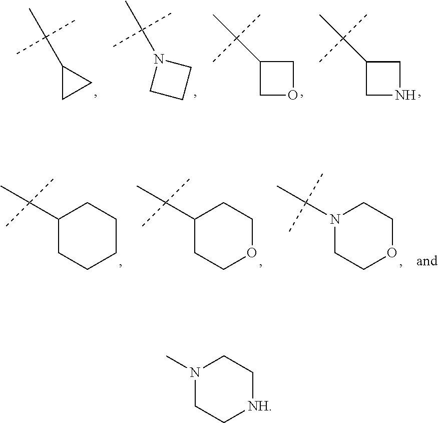

In some embodiments, R^{9b} and R^{18a} are independently selected from H, F, Cl, CH₃, CF₃, CH₂CF₃, CH₂C(CH₃)₃, CH₂CF(CH₃)₃,

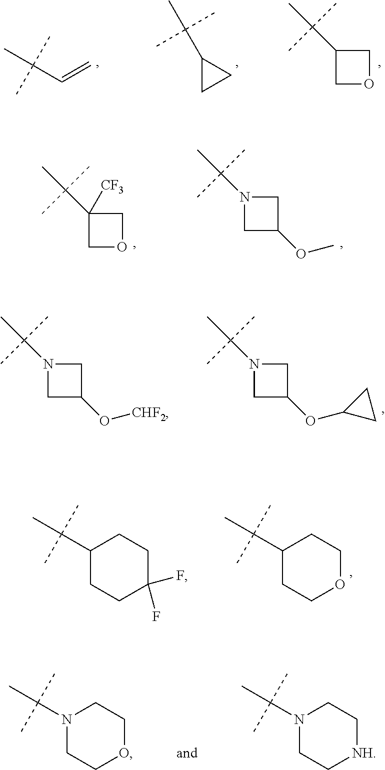

In some embodiments, two of independent R^{9a}, R^{9b}, R^{9′} and R^{9″}, together with the atoms to which they are connected, can optionally form an optionally substituted C₃-C₁₂ membered cycloalkyl ring, or an optionally substituted 3-12 membered heterocyclyl ring. In some embodiments, R^{9′} and R^{9″}, together with the atoms to which they are connected, can optionally form an optionally substituted C₃-C₁₂ membered cycloalkyl ring, or an optionally substituted 3-12 membered heterocyclyl ring. In some embodiments, two of each R^{18a}, R^{18b}, R^{18′} and R^{18″}, together with the atoms to which they are connected, can optionally form a C₃-C₁₂ membered cycloalkyl ring, or a 3-12 membered heterocyclyl ring. In some embodiments, R^{18″} and R^{18″}, together with the atoms to which they are connected, can optionally form a C₃-C₁₂ membered cycloalkyl ring, or a 3-12 membered heterocyclyl ring.

In some embodiments, R⁹ is selected from

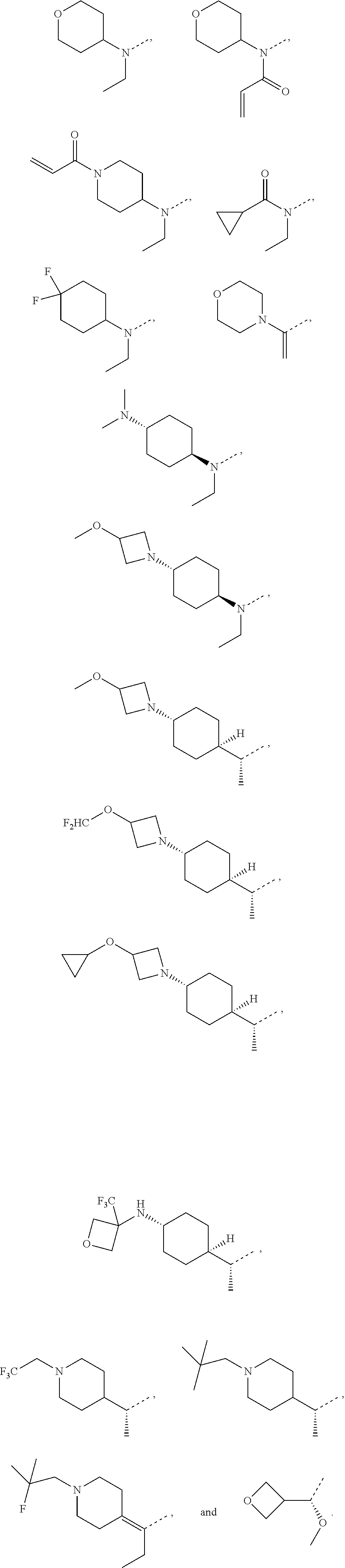

In some embodiments, R¹⁸ is selected from

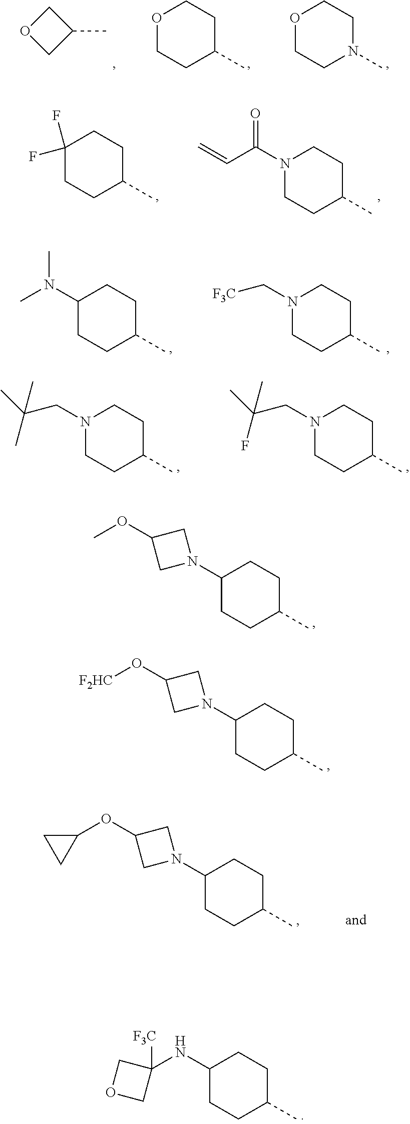

In some embodiments, R¹⁸ is selected from

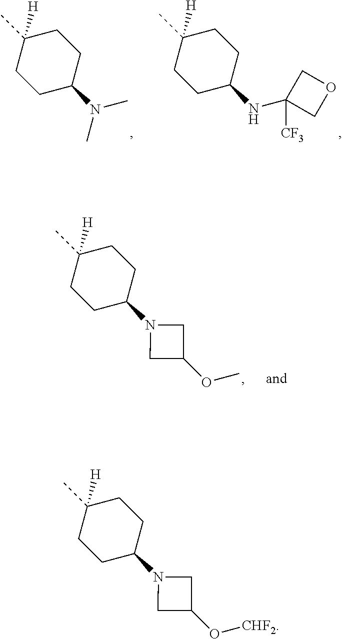

In some embodiments, R¹⁰ is selected from hydrogen, halogen, optionally substituted C₁-C₈ alkyl, and optionally substituted C₁-C₈ alkoxy. In some embodiments, R¹⁰ is H. In some embodiments, L^{11a} and L^{11b} are independently selected from null, —O—, —NR^{11′}—, —CR^{11′}R^{11″}—, —C(O)—, and —C(O)—NR^{11′}—. In some embodiments, L^{11a} is null, and L^{11b} is selected from null, —O—, —NR^{11′}—, —CR^{11′}R^{11″}—, —C(O)—, and —C(O)—NR^{11′}—. In some embodiments, L^{16b} is selected from null, —O—, —NR^{16′}—, —CR^{16′}R^{16″}—, —C(O)—, and —C(O)—NR^{16′}—. In some embodiments, L^{20a} and L^{20b} are independently selected from null, —O—, —NR^{20′}—, —CR^{20′}R^{20″}—, —C(O)—, and —C(O)—NR^{20′}—. In some embodiments, L²⁰a is null, and L^{20b} is selected from null, —O—, —NR^{20′}—, —CR^{20′}R^{20″}—, —C(O)—, and —C(O)—NR^{20′}—.

In some embodiments, R^{11′}, R^{11″}, R^{16′}, R^{16″}, R^{20′} and R^{20″}, at each occurrence, are independently selected from hydrogen, halogen, and optionally substituted groups consisting of C₁-C₈ alkyl, C₂-C₈ alkenyl, C₁-C₈ heteroalkyl, C₁-C₈ alkoxy, C₁-C₈ alkylamino, C₃-C₁₂ cycloalkyl, C₃-C₁₂ cycloalkoxy, C₃-C₁₂ cycloalkylamino, 3-12 membered heterocyclyl, 3-12 membered heterocyclyloxy, and 3-12 membered heterocyclylamino. In some embodiments, R^{11a} and R^{20a} are independently selected from null, and optionally substituted divalent groups consisting of C₁-C₈ alkylene, C₂-C₈ alkenyl, C₂-C₈ alkynylene, C₃-C₁₂ membered cycloalkylene, 3-12 membered heterocyclylene, aryl, and heteroaryl.

In some embodiments, R^{11a} and R^{20a} are independently selected from null, —C≡C—, or optionally substituted groups consisting of

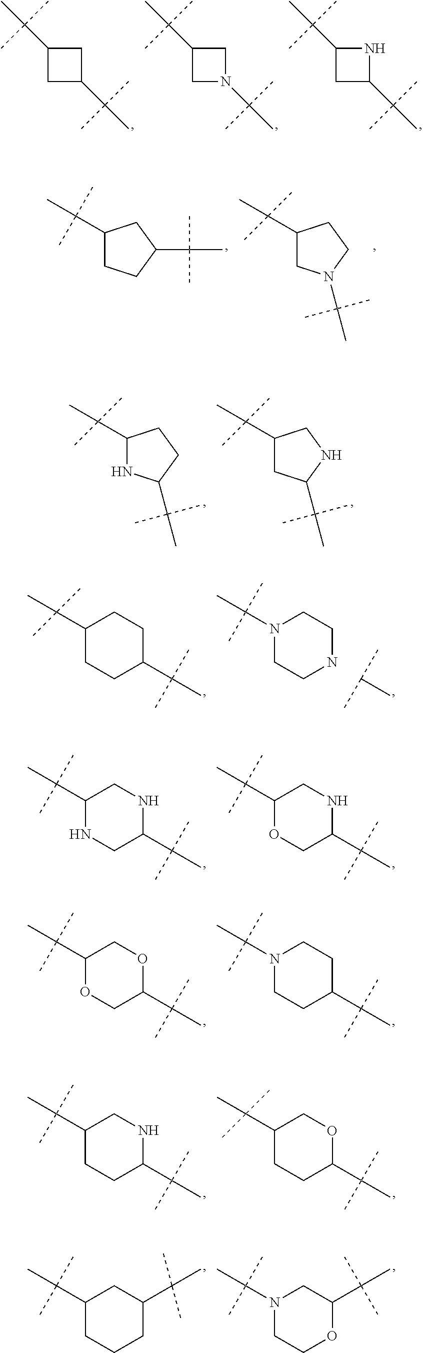

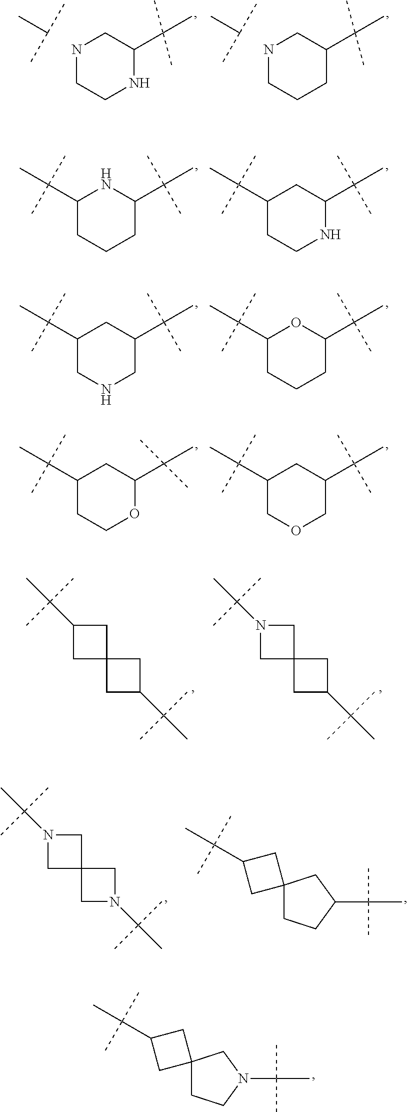

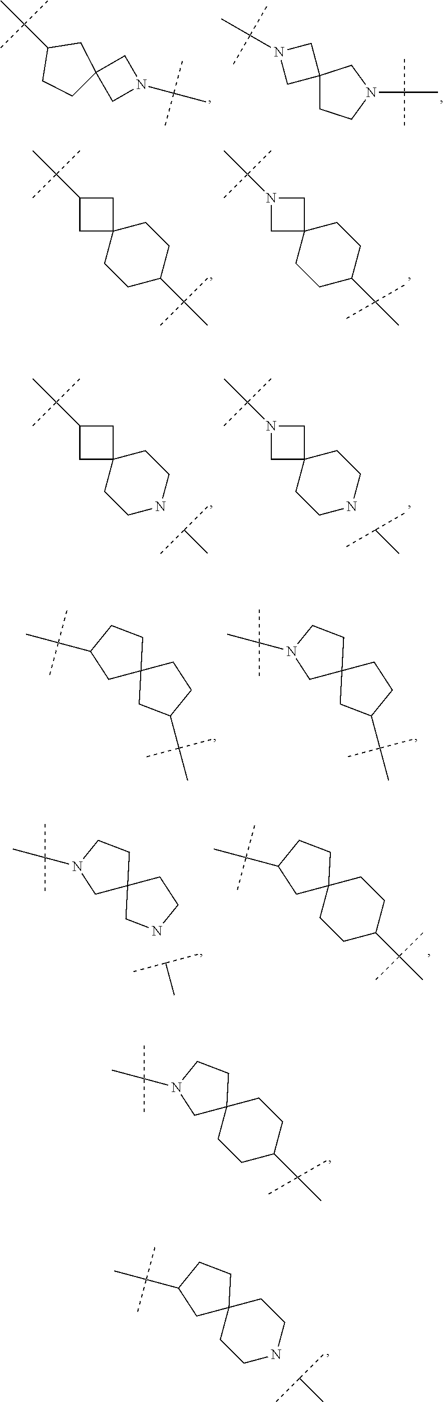

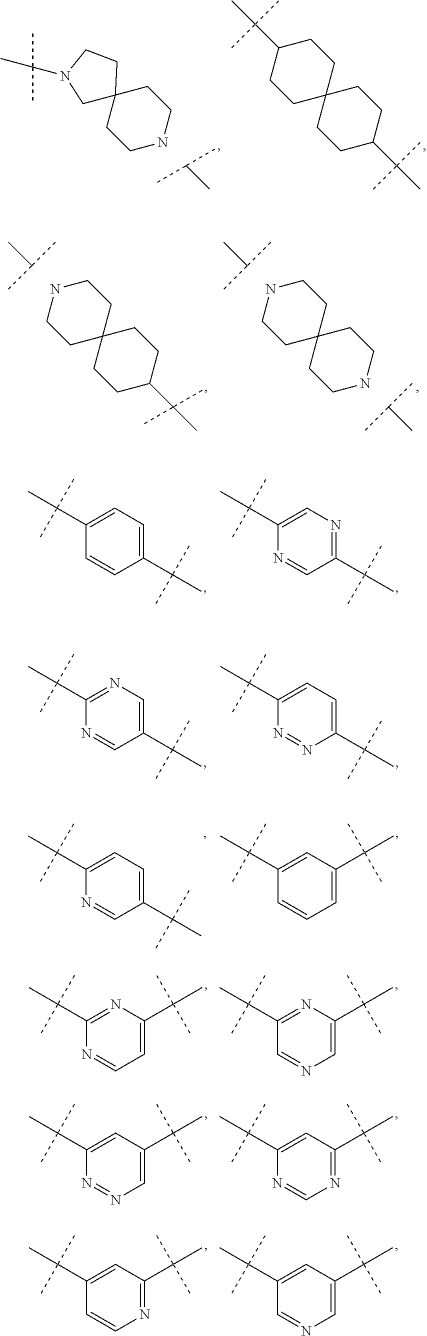

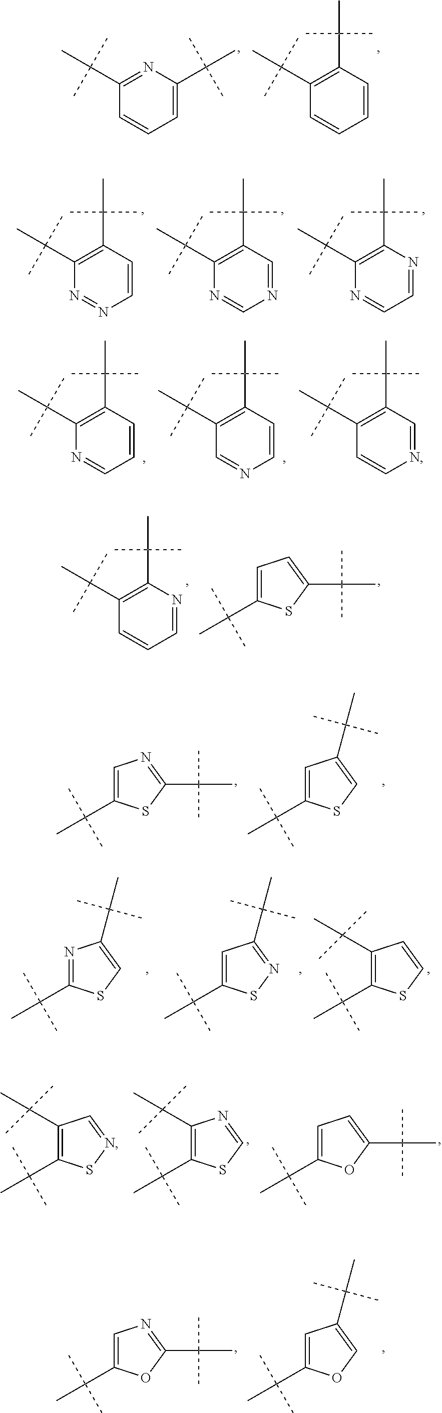

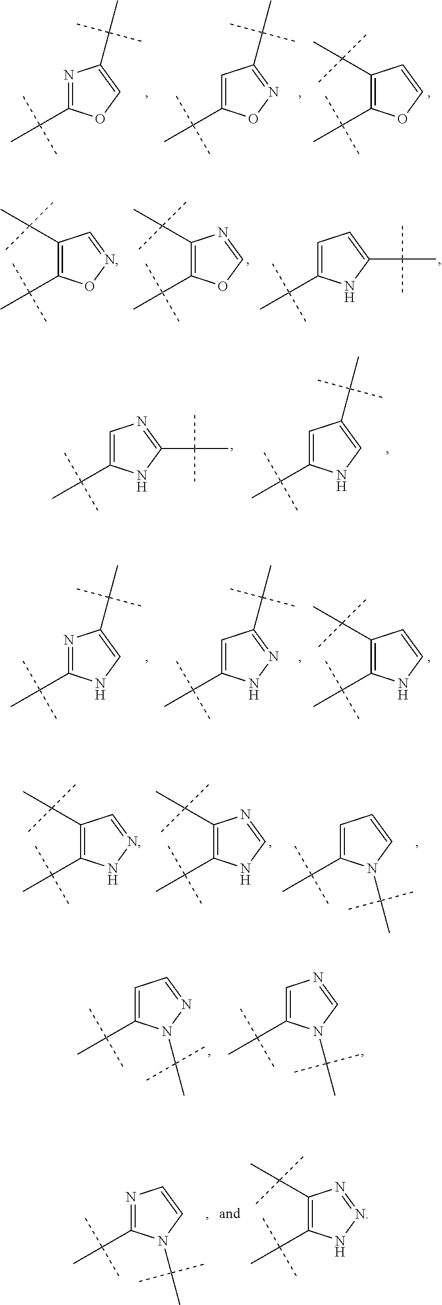

In some embodiments, R^{11a} and R^{20a} are independently selected from null, and optionally substituted divalent groups consisting of C₂-C₈ alkynylene, aryl, and heteroaryl.

In some embodiments, R^{11a} and R^{20a} are independently selected from null, —C≡C—, or optionally substituted groups consisting of

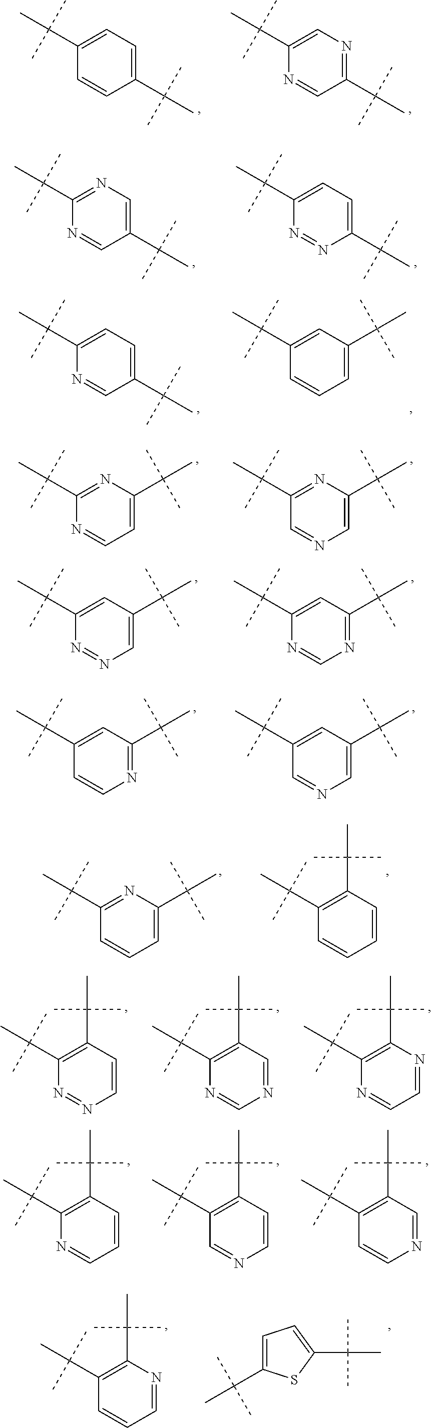

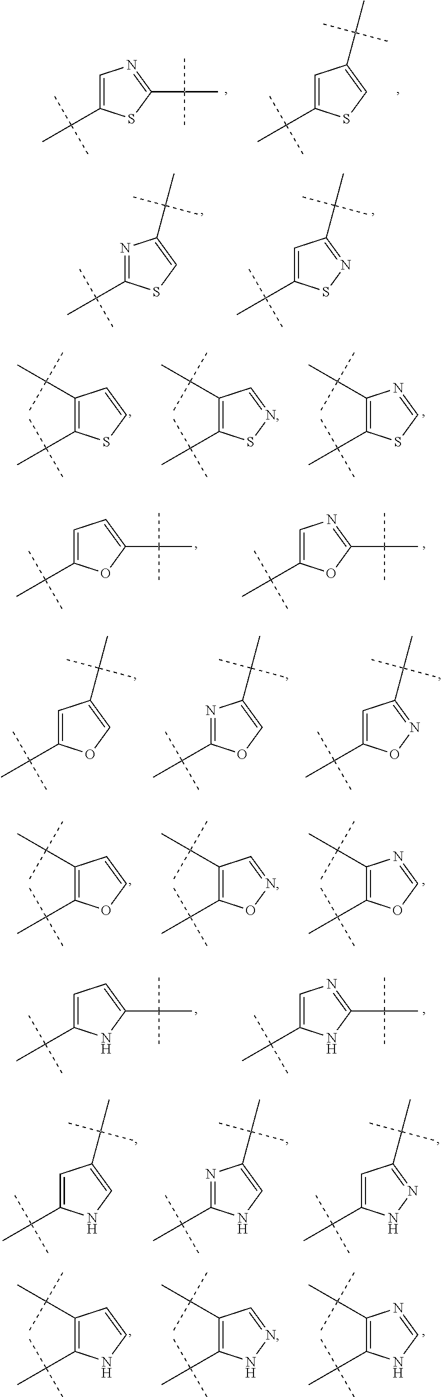

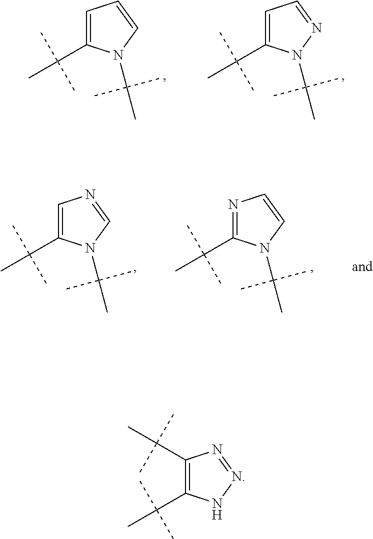

In some embodiments, R^{11a} and R^{20a} are independently selected from null, —C≡C—, or optionally substituted groups consisting of

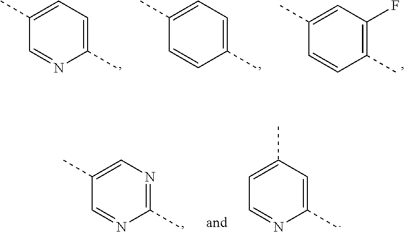

In some embodiments, R^{11b}, R^{16b} and R^{20b} are independently selected from hydrogen, halogen, cyano, and optionally substituted groups consisting of C₁-C₈ alkyl, C₂-C₈ alkenyl, C₂-C₈ alkynyl, C₃-C₁₂ membered cycloalkyl, 3-12 membered heterocyclyl, aryl, and heteroaryl. In some embodiments, R^{11b}, R^{16b} and R^{20b} are independently selected from hydrogen, halogen, cyano, and optionally substituted groups consisting of C₁-C₈ alkyl, C₃-C₁₂ membered cycloalkyl, and 3-12 membered heterocyclyl.

In some embodiments, R^{1b}, R^{16b} and R^{20b} are independently selected from H, F, Cl, Br, CN, or optionally substituted groups consisting of

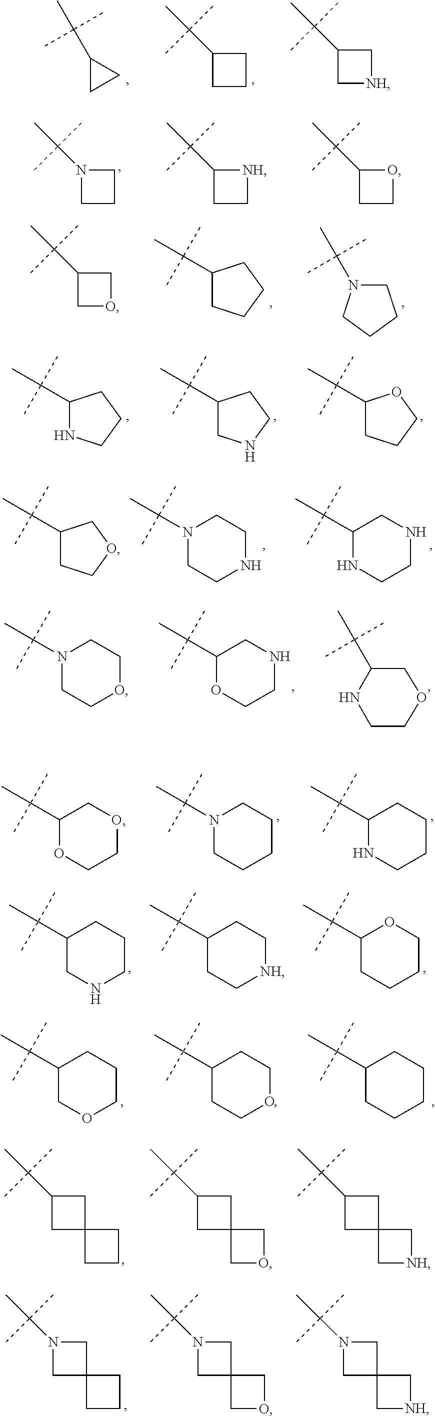

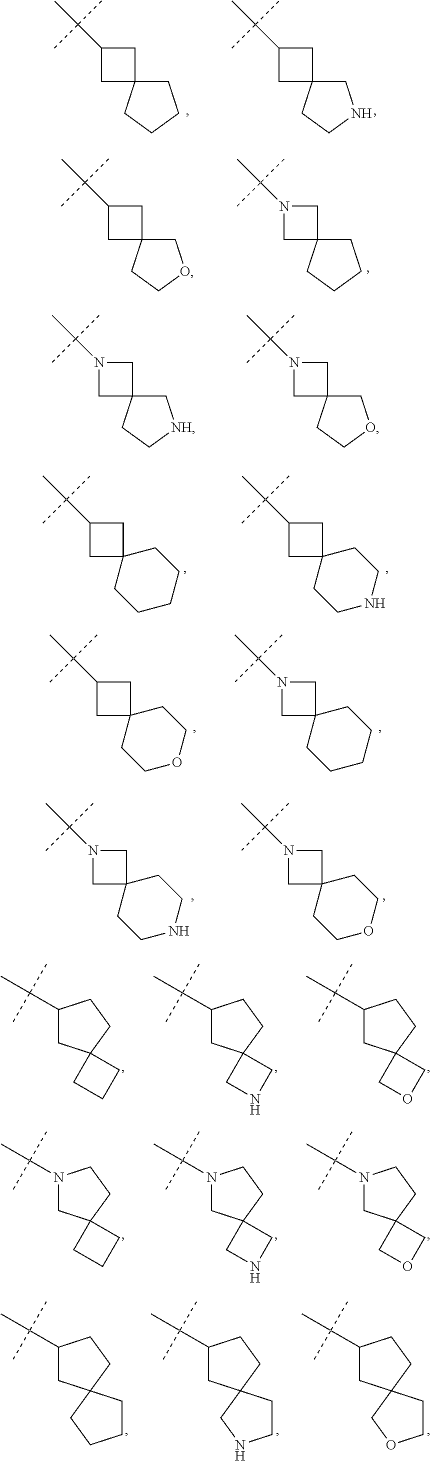

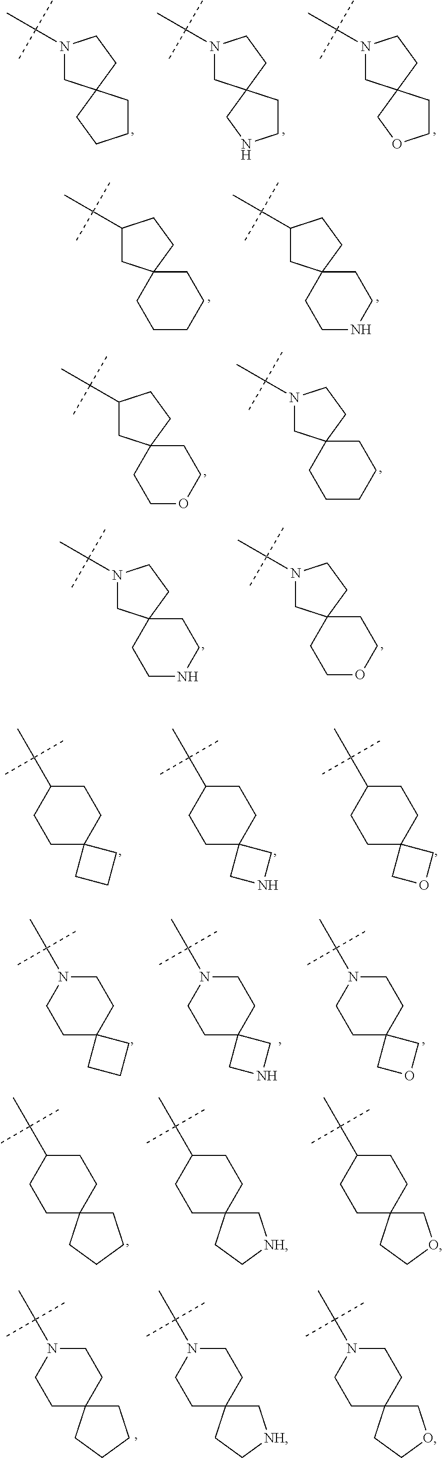

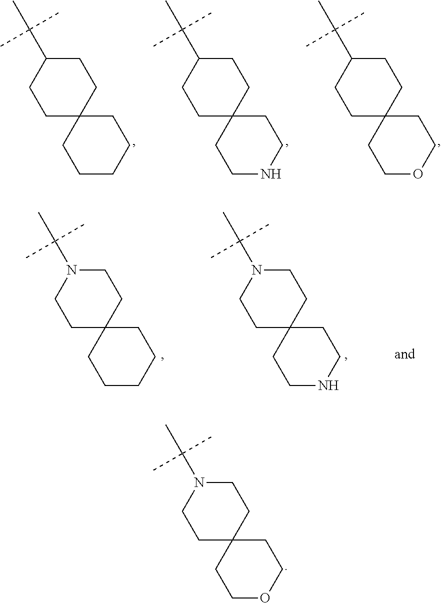

In some embodiments, R^{11b}, R^{16b} and R^{20b} are independently selected from H, F, Cl, Br, or optionally substituted groups consisting of

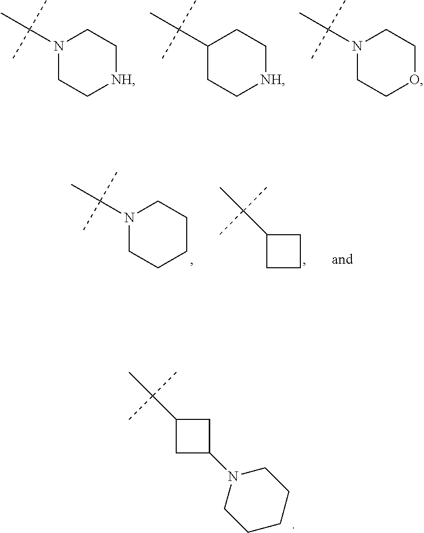

In some embodiments, R^{11b}, R^{16b} and R^{20b} are independently selected from H, F, Cl, Br, CN,

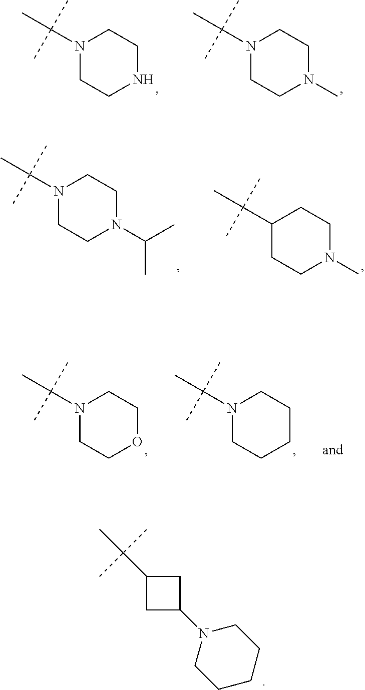

In some embodiments, R¹¹ and R²⁰ are independently selected from groups consisting of:

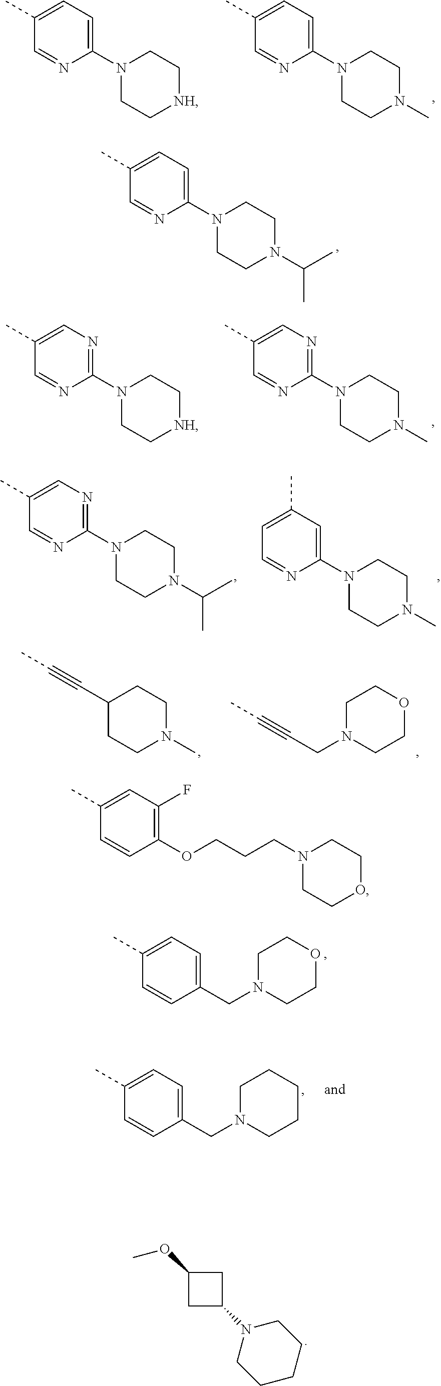

In some embodiments R¹⁶ is independently selected from groups consisting of

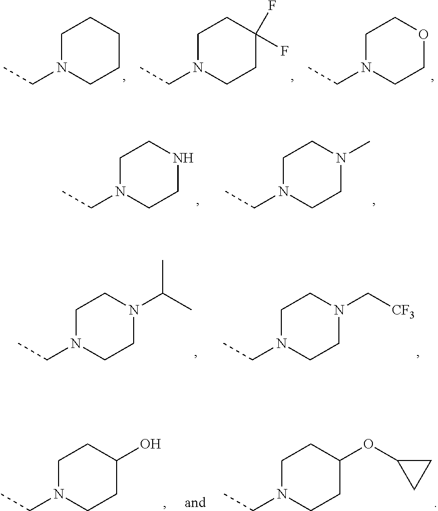

In some embodiments, R¹², R¹³, R¹⁴, R¹⁵, R¹⁹, R²², R²³, and R²⁵ are independently selected from hydrogen, halogen, optionally substituted C₁-C₈ alkyl, and optionally substituted C₁-C₈ alkenyl. In some embodiments, R¹², is selected from null and H. In some embodiments, R¹³, R¹⁴, R¹⁹, R²², and R²³ are independently selected from H and CH₃. In some embodiments, R¹⁵ is selected from CH₃, CH(CH₃)₂, and

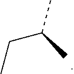

In some embodiments, R²⁵, and each occurrence, is selected from H and F. In some embodiments, R¹⁶, R¹⁷ and R¹⁸ is selected from optionally substituted C₁-C₈ alkyl, optionally substituted C₁-C₈ heteroalkyl, optionally substituted C₃-C₁₂ cycloalkyl, optionally substituted C₃-C₁₂ heterocycloalkyl, and optionally substituted 3-12 membered heterocyclyl. In some embodiments, R¹⁶

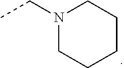

In some embodiments, R¹⁷ is optionally substituted C₁-C₈ alkyl. In some embodiments, R¹⁷ is CH₃. In some embodiments, R¹⁸ is optionally substituted C₃-C₁₂ cycloalkyl, optionally substituted C₃-C₁₂ heterocycloalkyl, and optionally substituted 3-12 membered heterocyclyl.

In some embodiments, R¹⁸ is selected from groups consisting of

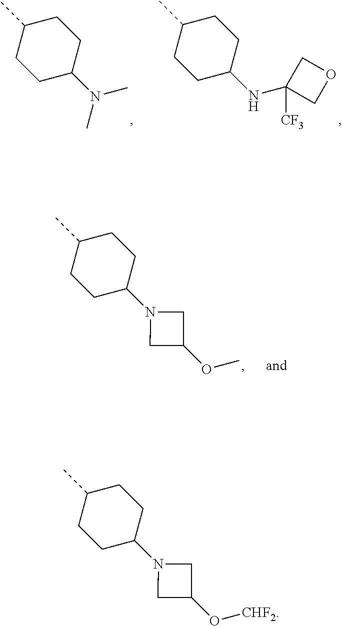

In some embodiments, R¹⁸ is selected from groups consisting of

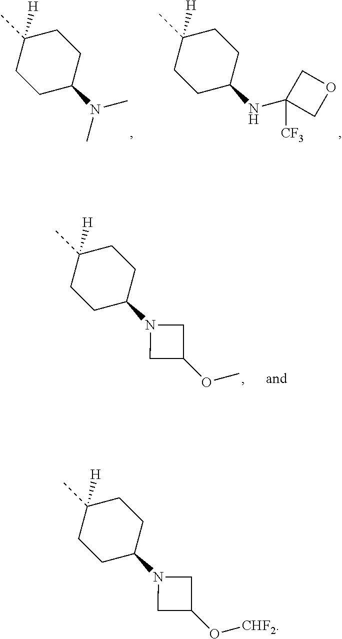

In some embodiments, R²¹ and R²⁴ are independently selected from optionally substituted C₁-C₈ alkyl, optionally substituted C₁-C₈ heteroalkyl, optionally substituted C₃-C₁₂ cycloalkyl, optionally substituted C₃-C₁₂ heterocycloalkyl, and optionally substituted 3-12 membered heterocyclyl, optionally substituted C₃-C₁₂ cycloalkyl, optionally substituted C₃-C₁₂ heterocycloalkyl, and optionally substituted 3-12 membered heterocyclyl, optionally substituted aryl, and optionally substituted heteroaryl. In some embodiments, R²¹ is selected from optionally substituted aryl, and optionally substituted heteroaryl. In some embodiments, R²¹ is

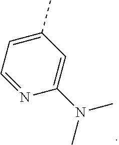

In some embodiments, R²⁴ is selected from optionally substituted C₁-C₈ alkyl. In some embodiments, R²⁴ is CH₃.

In some embodiments, the first compound (e.g., an EZH2 inhibitor) is selected from the group consisting of

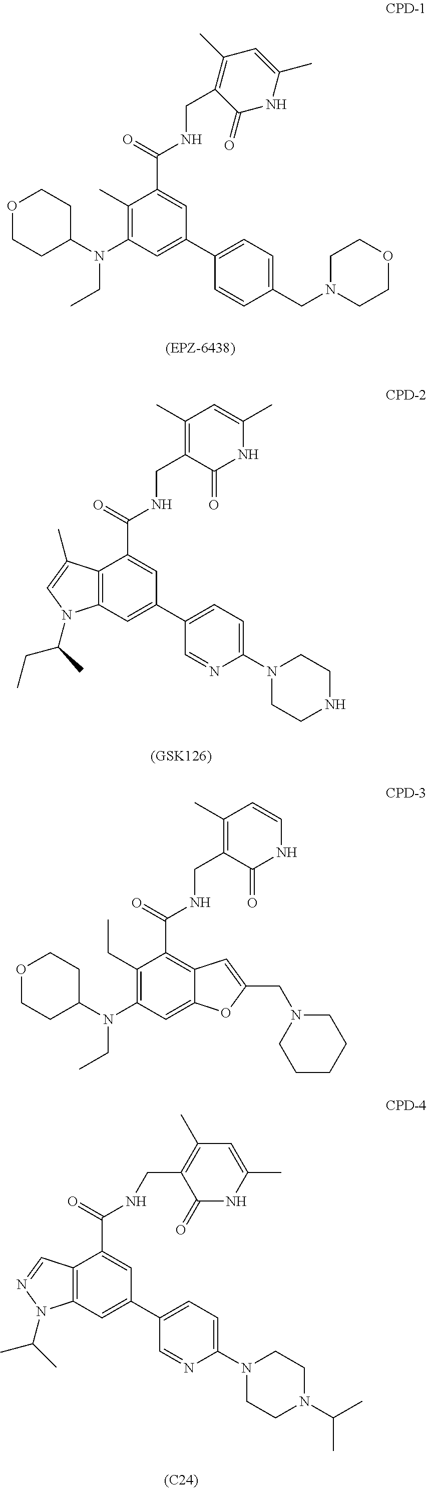 [SMILES: CCN(c1cc(-c2ccc(CN3CCOCC3)cc2)cc(C(=O)NCc2c(C)cc(C)[nH]c2=O)c1C)C1CCOCC1 ; CC[C@H](C)n1cc(C)c2c(C(=O)NCc3c(C)cc(C)[nH]c3=O)cc(-c3ccc(N4CCNCC4)nc3)cc21 ; CCc1c(N(CC)C2CCOCC2)cc2oc(CN3CCCCC3)cc2c1C(=O)NCc1c(C)cc[nH]c1=O ; Cc1cc(C)c(CNC(=O)c2cc(-c3ccc(N4CCN(C(C)C)CC4)nc3)cc3c2cnn3C(C)C)c(=O)[nH]1 ; "(C24)"]

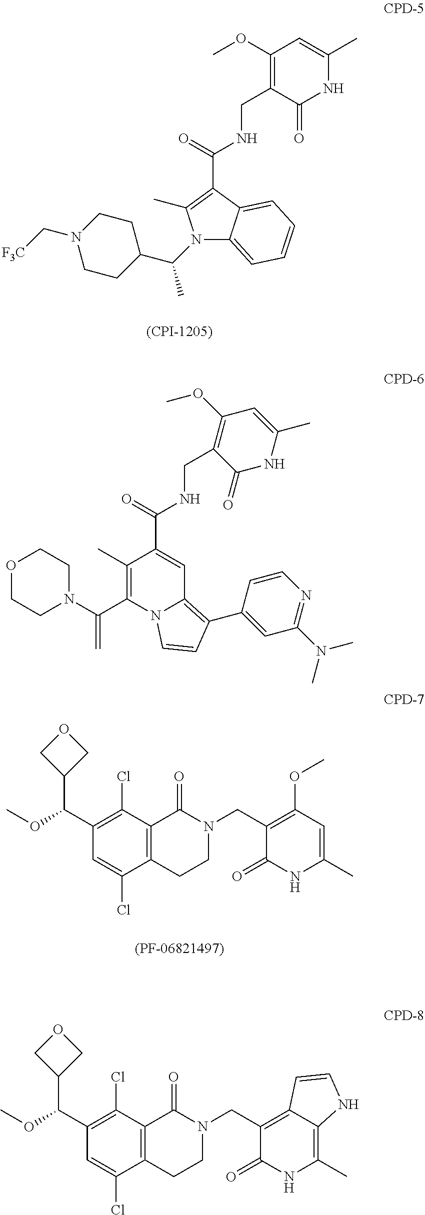

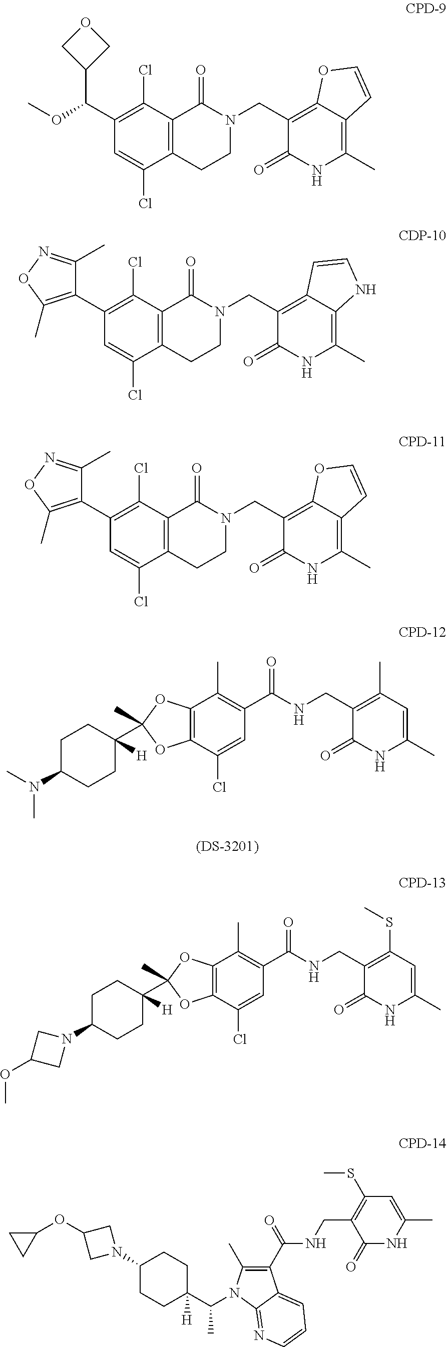

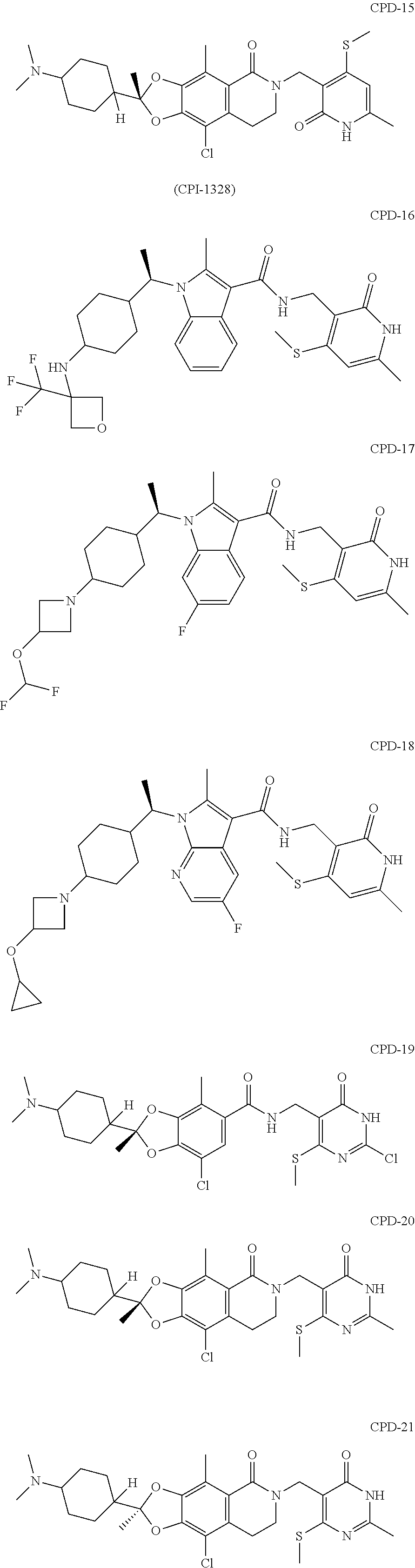

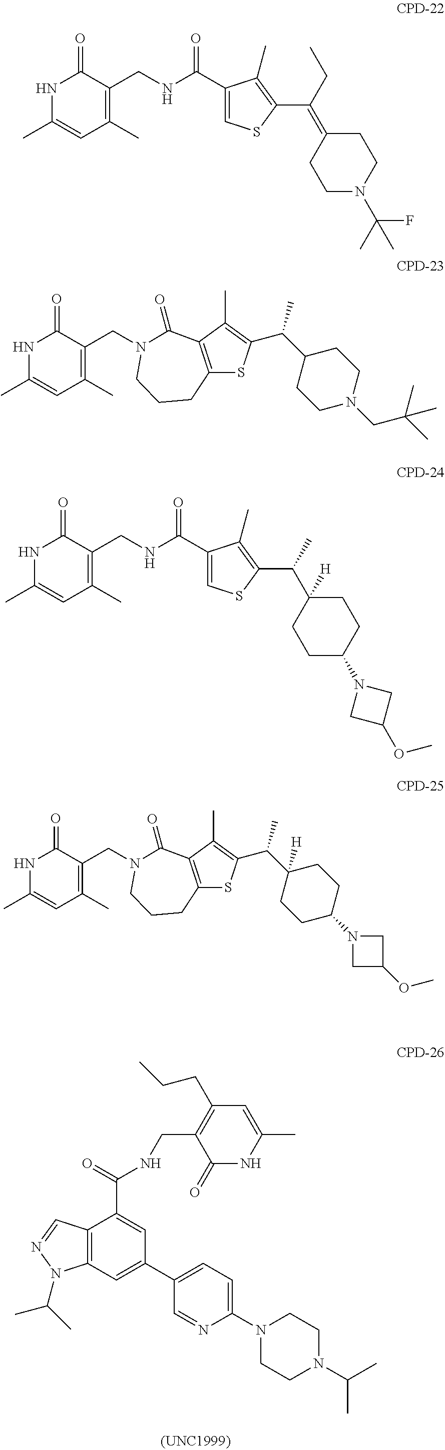

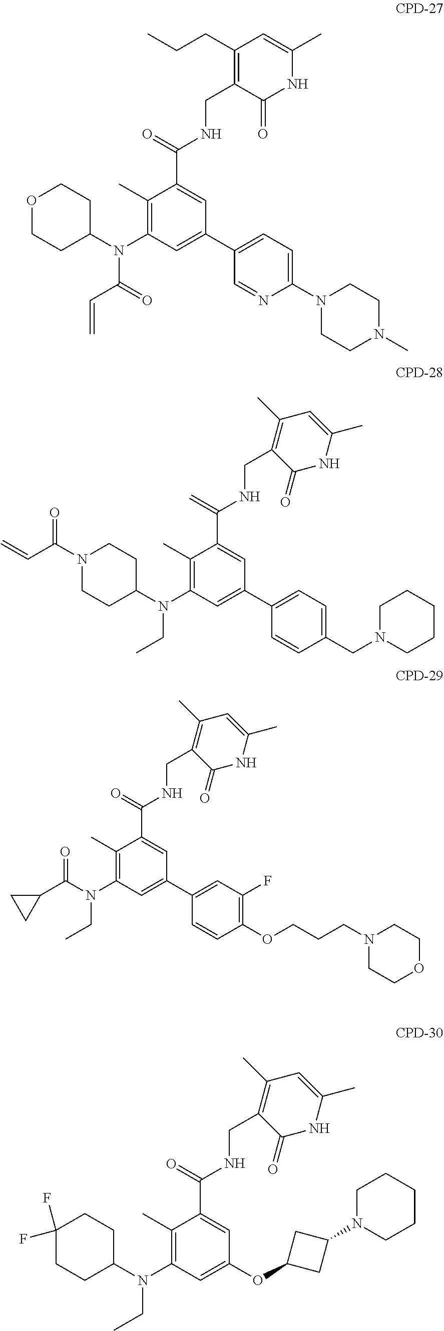

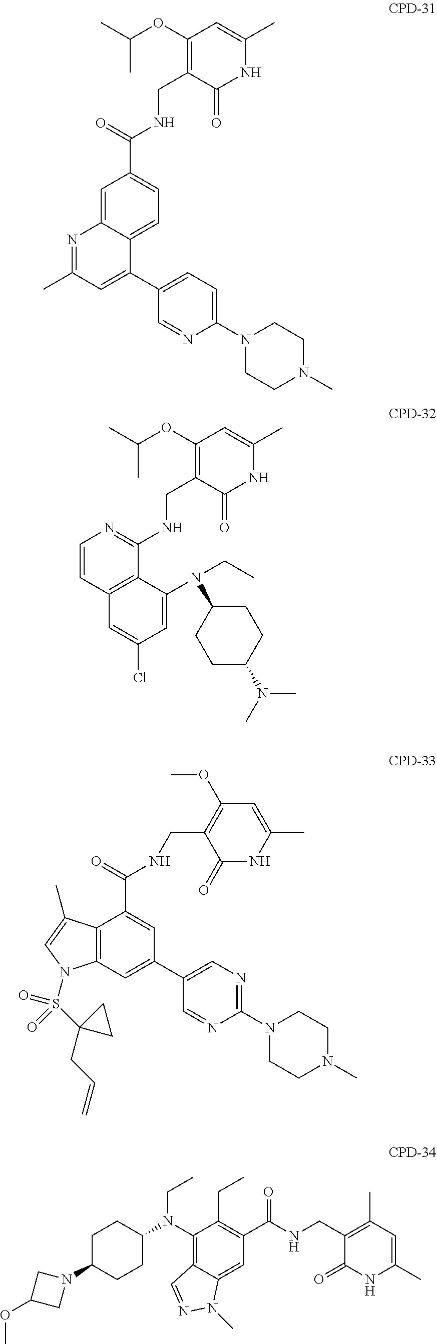

In some embodiments, the first compound is an EZH2 inhibitor. In some embodiments, the EZH2 inhibitor is selected from the group consisting of EPZ-6438 (tazemetostat), CPI-1205 (lirametostat), C24 (N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; CAS No. 1979157-17-9), CPI-0209 ((2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d][1,3]dioxole-5-carboxamide; CAS No. 2567686-02-4), CPI-1328 ((R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; CAS No. 2390367-27-6), PF-06821497 ((S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; CAS No. 1844849-10-1), DS-3201 (valemetostat), UNC1999 (1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; CAS No. 1431612-23-5), SHR2254 (N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; CAS No. 2098545-98-1), and GSK126 ((S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; CAS No. 1346574-57-9), or a pharmaceutically acceptable salt thereof.

In some embodiments, the first compound (e.g., EZH2 inhibitor) is EPZ-6438, CPI-1205, CPI-0209, DS-3201, PF-06821497, SHR2554, GSK126, CPI-1328, C24, or UNC1999. In some embodiments, the first compound is EPZ-6438, GSK126, CPI-1205, C24, CPI-0209, or SHR2554. In some embodiments, the first compound is EPZ-6438. In some embodiments, the first compound is CPI-1205. In some embodiments, the first compound is CPI-0209. In some embodiments, the first compound is DS-3201. In some embodiments, the first compound is PF-06821497. In some embodiments, the first compound is SHR2554. In some embodiments, the first compound is GSK126. In some embodiments, the first compound is CPI-1328. In some embodiments, the first compound is C24. In some embodiments, the first compound is UNC1999.

### Second Compound

Disclosed herein, in some embodiments, are methods, compositions, and combinations comprising a second compound. The second compound may be used in any of the methods described herein, such as in a method of treatment. The second compound may be included in a composition for use in a method of treatment. The second compound may be included in a combination for use in the treatment of cancer. In some embodiments, the second compound comprises an immunomodulatory drug. The immunomodulatory drug may include an immunomodulatory imide drug (IMiD). The IMiD may include a cereblon modulator such as a cereblon inhibitor. In some embodiments, the cereblon modulator is an IKZF1 and/or IZKF3 degrader or modulator. In some embodiments, the cereblon modulator is an IKZF1/3 degrader or modulator. In some embodiments, the IMiD is an IKZF1/3 degrader or modulator.

In some embodiments, the immunomodulatory drug is included in a method of treatment, or in a composition with a first compound.

Cereblon (CRBN) may include a protein encoded by the CRBN gene. In some embodiments, the IMiD comprises a CRBN modulator. The CRBN modulator may bind CRBN. In some embodiments, the second compound (e.g., a cereblon modulator) comprises a compound of Formula II:

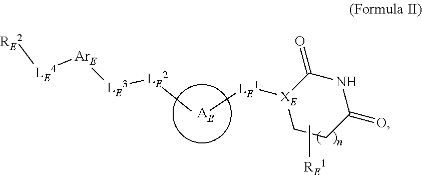

or a pharmaceutically acceptable salt, hydrate, solvate, prodrug, stereoisomer, tautomer, or analog thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring; L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}⁷ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring; Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

In some embodiments, ring A_{E} is a divalent group selected from groups consisting of Formulae A_{E}¹, A_{E}², A_{E}³, A_{E}⁴, A_{E}⁵, A_{E}⁶ and A_{E}⁷.

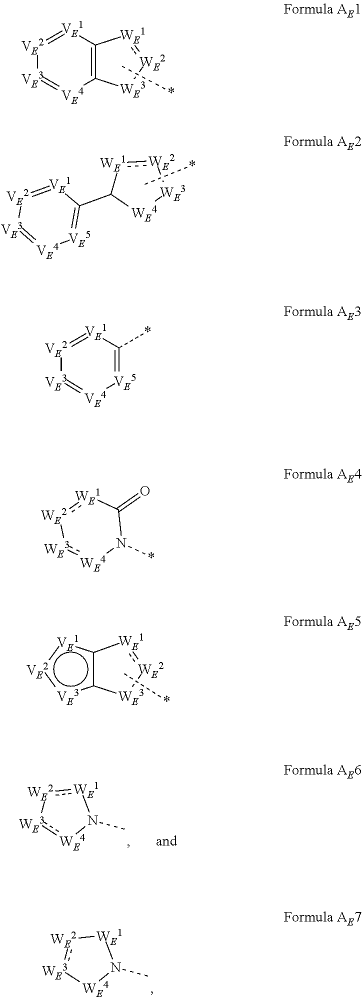

wherein * indicates the attachment to L_{E}¹, or X_{E} when L_{E}¹ is null; L_{E}² is attached to any possible position on the Ring A_{E}; [symbol] indicates a single bond or a double bond; V_{E}¹, V_{E}², V_{E}³, V_{E}⁴ and V_{E}⁵, at each occurrence, are each independently selected from the group consisting of a bond, C, CR_{E″}, S, N, and NR_{E}¹¹; or V_{E}¹ and V_{E}², V_{E}² and V_{E}³, V_{E}³ and V_{E}⁴, or V_{E}⁴ and V_{E}⁵ are combined together to optionally form optionally substituted aryl ring or optionally substituted heteroaryl ring; R_{E″}, at each occurrence, is independently selected from the group consisting of absent, hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or R_{E}¹¹ and another R_{E}¹¹ together with the atom(s) to which they are connected form optionally substituted C₃-C₁₂ cycloalkyl, optionally substituted 3-12 membered heterocyclyl ring, optionally substituted aryl, and optionally substituted heteroaryl; and W_{E}¹, W_{E}², W_{E}³ and W_{E}⁴ are each independently selected from the group consisting of —N═, —C—, —CR_{E}¹²=, —CO—, —O—, —CR_{E}¹²R_{E}¹³-, —NR_{E}¹²-, —CR_{E}¹²=CR_{E}¹³-, —N═CR_{E}¹²-, and N═N—; or W_{E}¹ and W_{E}², W_{E}² and W_{E}³, or W_{E}³ and W_{E}⁴ are combined together to optionally form optionally substituted aryl ring or optionally substituted heteroaryl ring, wherein R_{E}¹² and R_{E}¹³ at each occurrence, are independently selected from the group consisting of absent, hydrogen, halogen, cyano, nitro, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or R_{E}¹² and R_{E}¹³, on the same atom or on the adjacent atoms, together with the atom(s) to which they are connected optionally form an optionally substituted C₃-C₁₂ cycloalkyl, optionally substituted 3-12 membered heterocyclyl ring, optionally substituted aryl, and optionally substituted heteroaryl.

In some embodiments, V_{E}¹, V_{E}², V_{E}³, V_{E}⁴ and V_{E}⁵, at each occurrence, are each independently selected from the group consisting of a bond, C, CR_{E}¹¹, S, N, and NR_{E}¹¹; or V_{E}¹ and V_{E}², V_{E}² and V_{E}³, V_{E}³ and V_{E}⁴, or V_{E}⁴ and V_{E}⁵ are combined to optionally form optionally substituted aryl ring or optionally substituted heteroaryl ring.

In some embodiments, Ring A_{E} is a group consisting of Formula A_{E}¹, and wherein V_{E}¹, V_{E}², V_{E}³, and V_{E}⁴ are each independently selected from the group consisting of a bond, C, CR_{E}¹¹, S, N, and NR_{E}¹¹.

In some embodiments, Ring A_{E} is a group consisting of Formula A_{E}², and wherein V_{E}¹, V_{E}², V_{E}³, V_{E}⁴ and V_{E}⁵, at each occurrence, are each independently selected from the group consisting of a bond, C, CR_{E}¹¹, S, N, and NR_{E}¹¹.

In some embodiments, Ring A_{E} is a group consisting of Formula A_{E}³, and wherein V_{E}¹, V_{E}², V_{E}³, V_{E}⁴ and V_{E}⁵ are each independently selected from the group consisting of a bond, C, CR_{E″}, S, N, and NR_{E}¹¹; or V_{E}¹ and V_{E}², V_{E}² and V_{E}³, V_{E}³ and V_{E}⁴, or V_{E}⁴ and V_{E}⁵ are combined together to optionally form optionally substituted aryl ring or optionally substituted heteroaryl ring.

In some embodiments, Ring A_{E} is a group consisting of Formula A_{E}⁴, and wherein [symbol] is a single bond and W_{E}¹, W_{E}², W_{E}³ and W_{E}⁴ are each independently selected from the group consisting of —CO—, —O—, —CR_{E}¹²R_{E}¹³-, and —NR_{E}¹²-.

In some embodiments, Ring A_{E} is a group consisting of Formula A_{E}⁴, and wherein [symbol] is a double bond and W_{E}¹, W_{E}², W_{E}³ and W_{E}⁴ are each independently selected from the group consisting of —N═, and —CR_{E}¹²-.

In some embodiments, Ring A_{E} is a group consisting of Formula A_{E}⁵, and wherein V_{E}¹, V_{E}², and V_{E}³ are each independently selected from the group consisting of CR_{E}¹¹, S, N, and NR_{E}¹¹, with the proviso that at least one of V_{E}¹, V_{E}², and V_{E}³ is S, N or NR_{E}¹¹.

In some embodiments, Ring A_{E} is a group consisting of Formula A_{E}¹, A_{E}², and A_{E}⁵, and W_{E}¹, W_{E}², W_{E}³ and W_{E}⁴ are each independently selected from the group consisting of —N═, —CR_{E}¹²═, —CO—, —O—, —S—, —CR_{E}¹²R_{E}¹³-, and —NR_{E}¹²-.

In some embodiments, Ring A_{E} is a group consisting of Formula A_{E}⁶, and wherein [symbol] is a double bond and W_{E}¹, W_{E}², W_{E}³ and W_{E}⁴ are each independently selected from the group consisting of —N═, —CR_{E}¹²═.

In some embodiments, Ring A_{E} is a group consisting of Formula A_{E}⁷, and wherein [symbol] is a double bond and W_{E}¹ and W_{E}⁴ are independently selected from —CO—, and CR_{E}¹²R_{E}¹³-; and W_{E}² and W_{E}³ are independently selected from the group consisting of —N═, and —CR_{E}¹²═.

In some embodiments, Ring A_{E} is a group consisting of Formula A_{E}⁷, and wherein [symbol] is a single bond and W_{E}¹, W_{E}², W_{E}³ and W_{E}⁴ are each independently selected from the group consisting of —CO—, —O—, —CR_{E}¹²R_{E}¹³-, and —NR_{E}¹²-.

In some embodiments, R_{E}¹¹, at each occurrence, is selected from hydrogen, halogen, cyano, nitro, hydroxy, amino, optionally substituted C₁-C₈ alkyl, optionally substituted C₁-C₈ heteroalkyl, optionally substituted C₁-C₈ alkoxyl, optionally substituted C₁-C₈ alkylamino, optionally substituted C₃-C₁₂ cycloalkyl, and optionally substituted 3 to 12 membered heterocyclyl, or two independent R_{E}¹¹ groups together with the atom(s) to which they are connected optionally form C₃-C₁₂ cycloalkyl, 3-12 membered heterocyclyl, optionally substituted aryl, and optionally substituted heteroaryl.

In some embodiments, R_{E}¹¹, at each occurrence, is selected from hydrogen, halogen, cyano, nitro, and optionally substituted C₁-C₈ alkyl, optionally substituted C₁-C₈ heteroalkyl, optionally substituted C₁-C₈ alkoxyl, optionally substituted C₃-C₁₂ cycloalkyl, and optionally substituted 3 to 12 membered heterocyclyl, or two independent R_{E}¹¹ groups together with the atom(s) to which they are connected optionally form optionally substituted C₃-C₁₂ cycloalkyl, optionally substituted 3-12 membered heterocyclyl, optionally substituted aryl, and optionally substituted heteroaryl. In some embodiments, R_{E}¹¹, at each occurrence, is selected from H, F, Cl, Me, OMe, OCF₃, O-iPr, or O-cPr. In some embodiments, two adjacent R_{E}¹¹ together with the atom(s) to which they are connected optionally form optionally substituted C₃-C₁₂ cycloalkyl, optionally substituted 3-12 membered heterocyclyl, optionally substituted aryl, and optionally substituted heteroaryl.

In some embodiments, R_{E}¹² and R_{E}¹³, at each occurrence, are independently selected from hydrogen, halogen, cyano, nitro, optionally substituted C₁-C₈ alkyl, optionally substituted C₁-C₈ heteroalkyl, optionally substituted C₃-C₁₂ cycloalkyl, and optionally substituted 3 to 12 membered heterocyclyl; or two independent R_{E}¹², two independent R_{E}¹³, or one R_{E}¹² and one R_{E}¹³, together with the atom(s) to which they are connected optionally form optionally substituted C₃-C₁₂ cycloalkyl, or optionally substituted 3-12 membered heterocyclyl.

In some embodiments, R_{E}¹² and R_{E}¹³, at each occurrence, are independently selected from H, F, or Me.

In some embodiments, two independent R_{E}¹², two independent R_{E}¹³, or one R_{E}¹² and one R_{E}¹³ together with the atom(s) to which they are connected optionally form optionally substituted C₃-C₁₂cycloalkyl, optionally substituted 3-12 membered heterocyclyl, optionally substituted aryl, and optionally substituted heteroaryl.

In some embodiments, ring A_{E} is a moiety selected from the group consisting of Formulae A_{E}¹-a, A_{E}¹-b, A_{E}¹-c, A_{E}¹-d, A_{E}²-a, A_{E}³-a, A_{E}³-b, A_{E}⁴-a, A_{E}⁴-b, A_{E}⁴-c, A_{E}⁵-a, A_{E}⁶-a, and A_{E}⁷-a:

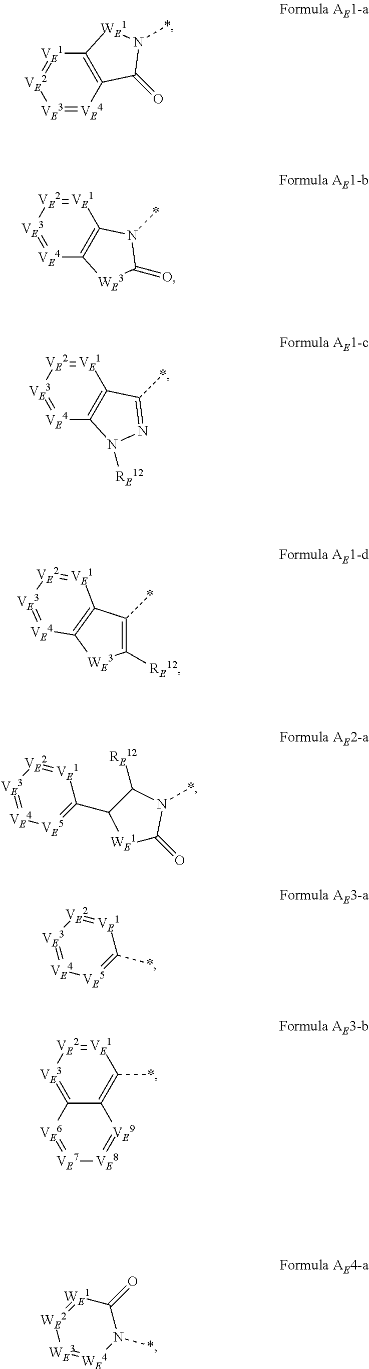

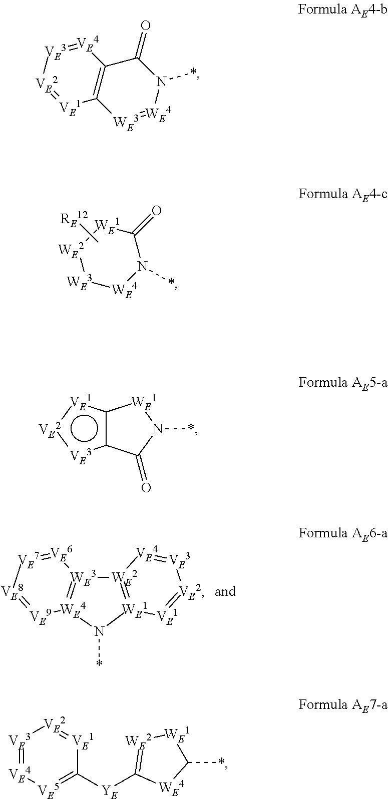

wherein * indicates the attachment to L_{E}¹, or X_{E} when L_{E}¹ is null; L_{E}² is attached to any possible position on the Ring A_{E}; V_{E}¹, V_{E}², V_{E}³, V_{E}⁴, V_{E}⁵, W_{E}¹, W_{E}², W_{E}³, W_{E}⁴, and R_{E}¹² are defined as in Formulae A_{E}¹, A_{E}², A_{E}³, A_{E}⁴, A_{E}⁵, A_{E}⁶ and A_{E}⁷; V_{E}⁶, V_{E}¹, V_{E}⁸, and V_{E}⁹ are each independently selected from a bond, C, CR_{E}¹⁴ and N; or V_{E}¹ and V_{E}², V_{E}² and V_{E}³, V_{E}³ and V_{E}⁴, or V_{E}⁴ and V_{E}⁵ are combined together to optionally form C₆ aryl ring or a 5, 6 or 7 membered heteroaryl ring; Y_{E} is selected from null, —CO—, —O—, —S—, —CR_{E}¹⁵R_{E}¹⁶- and —NR_{E}¹⁵-; and R_{E}¹⁴, R_{E}¹⁵, and R_{E}¹⁶, at each occurrence, is independently selected from the group consisting of hydrogen, halogen, cyano, nitro, hydroxy, amino, optionally substituted C₁-C₆ alkyl, optionally substituted C₂-C₆ alkenyl, optionally substituted C₂-C₆ alkynyl, optionally substituted 1-6 membered heteroalkyl, optionally substituted 2-6 membered heteroalkenyl, optionally substituted 2-6 membered heteroalkynyl, optionally substituted C₁-C₆ alkoxy, optionally substituted C₁-C₆ alkylamino, optionally substituted 3-8 membered carbocyclyl, and optionally substituted 3-8 membered heterocyclyl. In some embodiments, Y_{E} is selected from null, —CO—, —O—, —S—, —CH₂—, and —NH—. In some embodiments, Y_{E} is selected from —NH—.

In some embodiments, ring A_{E} is a moiety selected from the optionally substituted group consisting of

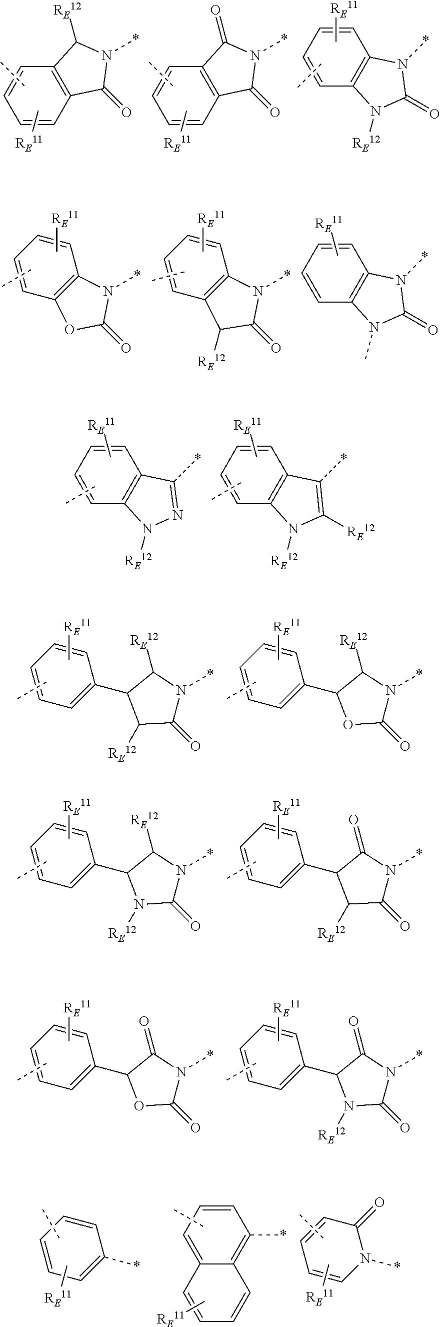

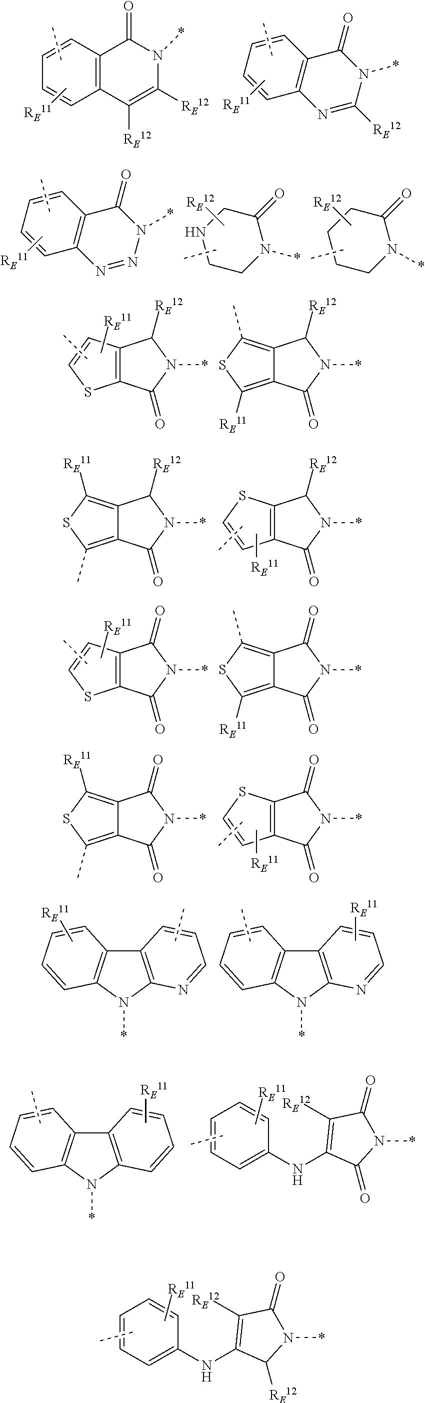

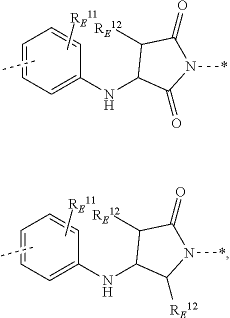

wherein Dashed bond with * indicates the attachment to L_{E}¹, or X_{E} when L_{E}¹ is null; dashed bond without * indicates the attachment to L_{E}²; and R_{E}¹¹ and R_{E}¹² is defined as in Formulae A_{E}¹, A_{E}², A_{E}³, A_{E}⁴, A_{E}⁵, A_{E}⁶ and A_{E}⁷. In some embodiments, Ring A_{E} is of Formulae A_{E}¹, A_{E}², A_{E}³, A_{E}⁴, A_{E}⁵, A_{E}⁶ and A_{E}⁷, and L_{E}¹ is null. In some embodiments, Ring A_{E} is of Formula A_{E}³, and L_{E}¹ is not null.

In some embodiments, the CRBN modulator is a compound consisting of

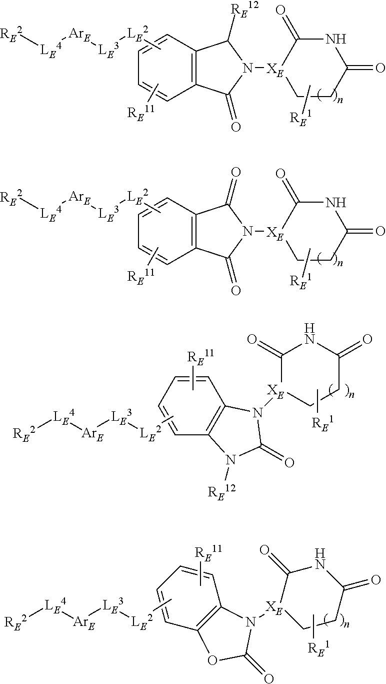

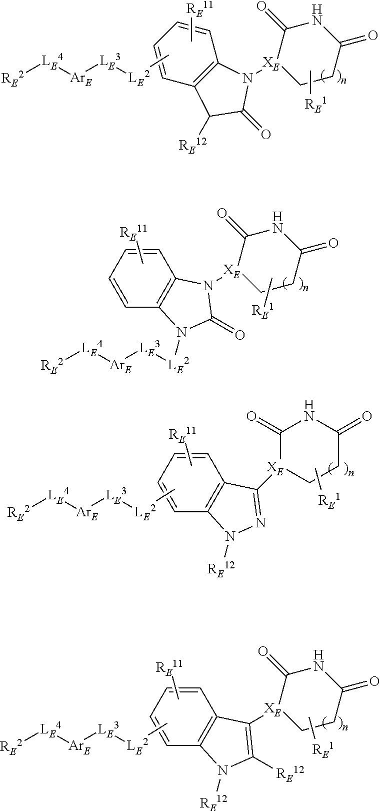

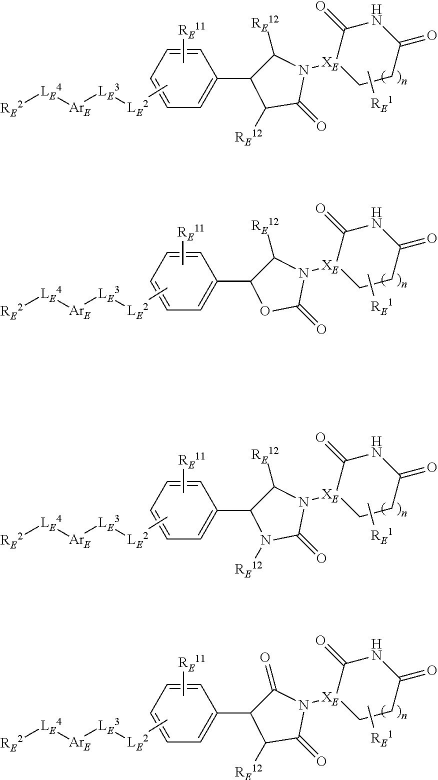

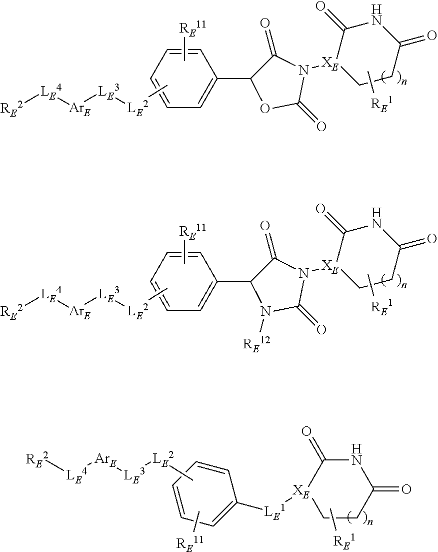

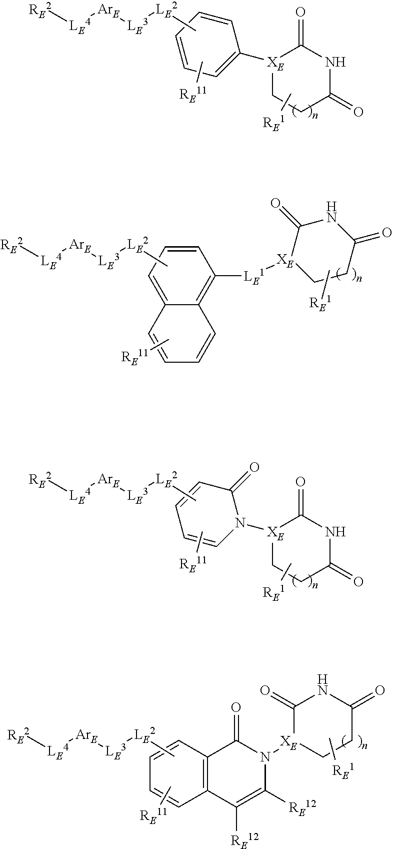

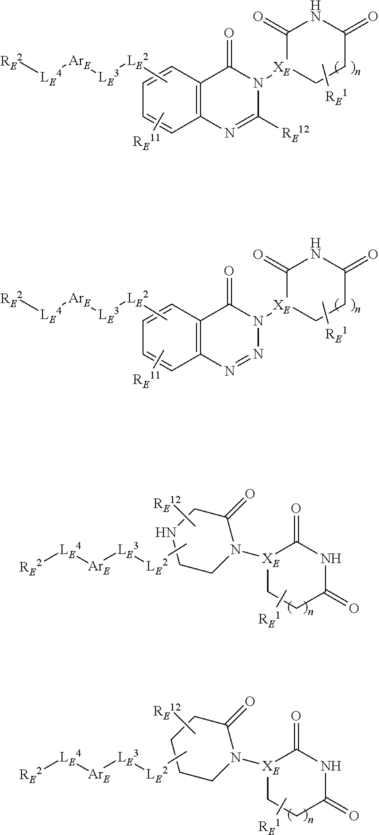

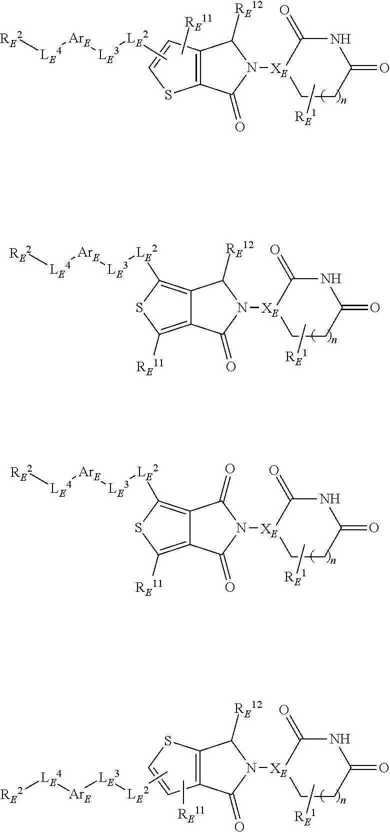

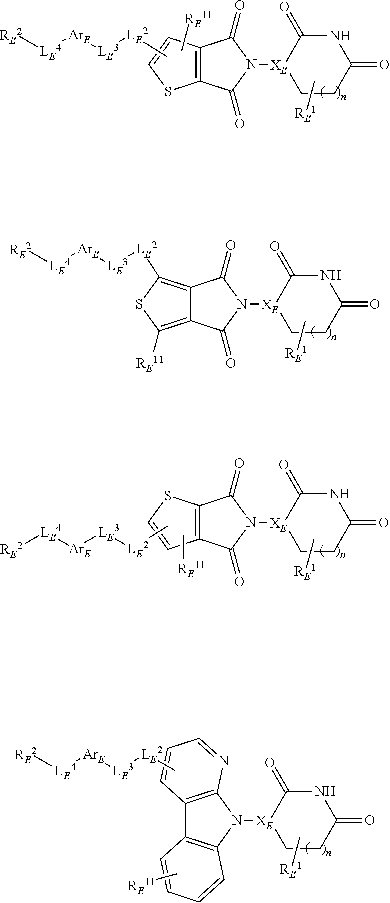

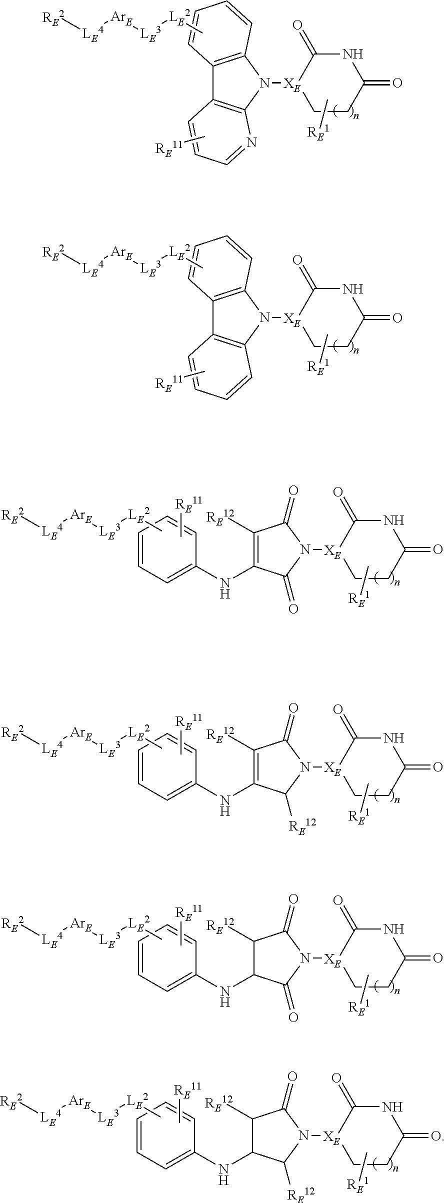

In some embodiments, X_{E} is CR_{E}¹. In some embodiments, X_{E} is CH. In some embodiments, X_{E} is N.

In some embodiments, the second compound (e.g., cereblon modulator) is a compound consisting of

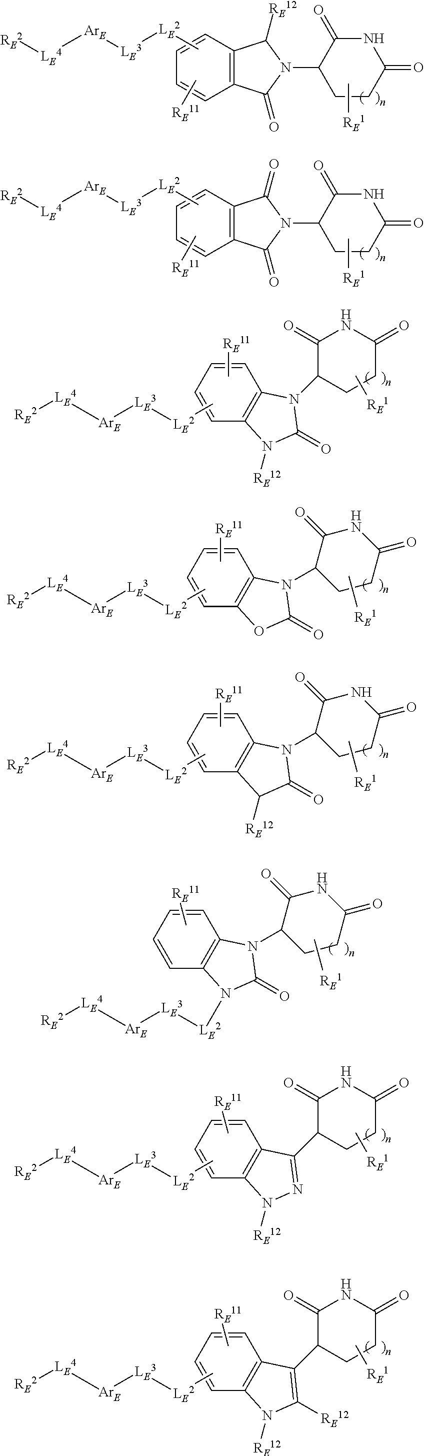

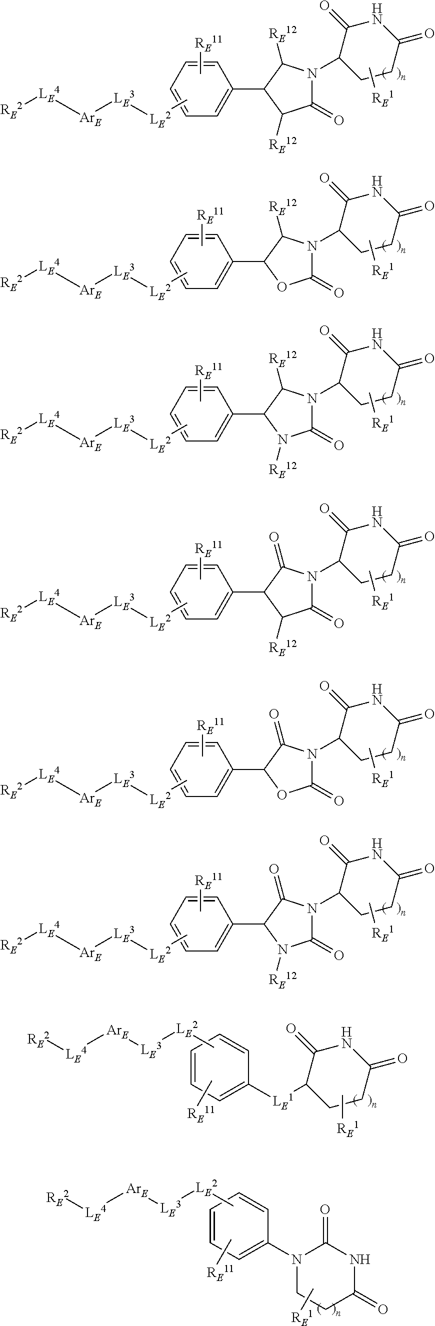

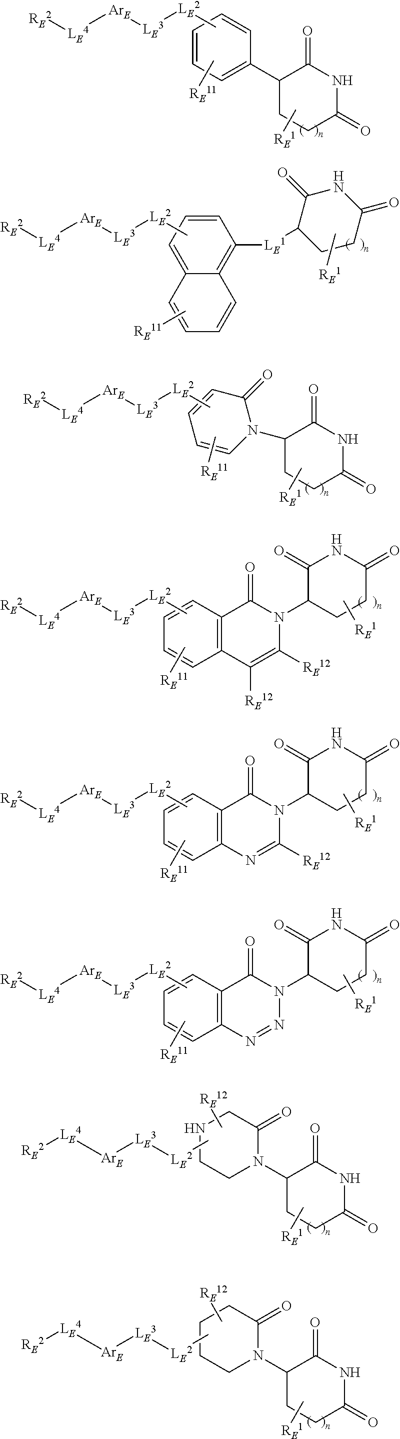

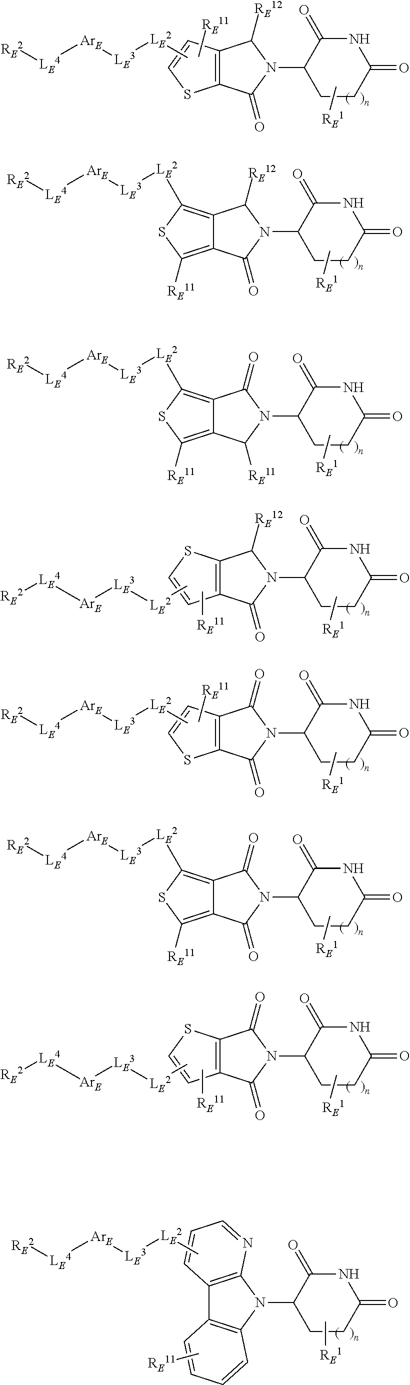

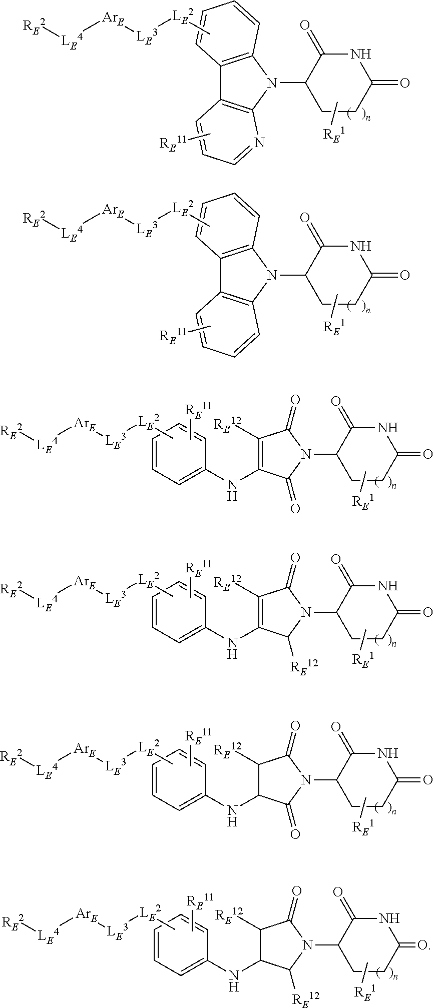

In some embodiments, L_{E}¹, L_{E}², L_{E}³ and L_{E}⁴ are divalent groups independently selected from the group consisting of -L_{E}^{a}-L_{E}^{b}-; wherein L_{E}^{a} and L_{E}^{b}, at each occurrence, are independently selected from the groups consisting of null, —CO—, —O—, —S—, —CR_{E}⁶R_{E}⁷- and —NR_{E}⁶-, with the proviso that -L_{E}^{a}-L_{E}b- is not —O—O—, wherein R_{E}⁶ and R_{E}¹ are independently selected from the group consisting of null, H, F, Cl, Br, CN, OH, NH₂, —NH(C₁-C₄ alkyl), —N(C₁-C₄ alkyl)₂, —NH(C₃-C₆ cycloalkyl), —N(C₁-C₄ alkyl)(C₃-C₆ cycloalkyl), and C₁-C₄ alkyl, C₁-C₄ heteroalkyl, or R_{E}⁶ and R_{E}⁷, together with the atoms to which they are connected, can optionally form a C₃-C₁₂ membered cycloalkyl ring, or a 3-12 membered heterocyclyl ring.

In some embodiments, L_{E}¹, L_{E}², L_{E}³ and L_{E}⁴ are divalent groups independently selected from the group consisting of -L_{E}^{a}-L_{E}^{b}-; wherein L_{E}^{a} and L_{E}^{b}, at each occurrence, are independently selected from the groups consisting of null, —CO—, —O—, —S—, —CR_{E}⁶R_{E}⁷- and —NR_{E}⁶-, with the proviso that -L_{E}^{a}-L_{E}b- is not —O—O—, wherein R_{E}⁶ and R_{E}¹ are independently selected from the group consisting of null, H, F, Cl, Br, CN, OH, NH₂, —NHCH₃, —N(CH₃)₂, —NHCF₃, —NH(iPr), —NH(cPr), CH₃, —CH₂CH₃, —CH₂CF₃, —CH₂CHF₂, -iPr, and -cPr, or R_{E}⁶ and R_{E}⁷, together with the atoms to which they are connected, can optionally form a C₃-C₆ membered cycloalkyl ring, or a 3-6 membered heterocyclyl ring.

In some embodiments, L_{E}¹, L_{E}², L_{E}³ and L_{E}⁴ are divalent groups independently selected from the group consisting of -L_{E}^{a}-L_{E}^{b}-; wherein L_{E}^{a} and L_{E}^{b}, at each occurrence, are independently selected from the groups consisting of null, —CO—, —O—, —S—, —NH—, —N(C₁-C₄ alkyl)-, —N(C₃-C₆ cycloalkyl)-, —CH₂—, —CH(C₁-C₄ alkyl)-, —CH(C₃-C₆ cycloalkyl)-, —C(C₁-C₄ alkyl)(C₁-C₄ alkyl)-, —C(C₁-C₄ alkyl)(C₃-C₆ cycloalkyl)-, —C(C₂-C₆ alkylene)-, and —C(C₂-C₆ heteroalkylene)-.

In some embodiments, L_{E}¹, L_{E}², L_{E}³ and L_{E}⁴ are divalent groups independently selected from the group consisting of null, —CONH—, —CO—N(C₁-C₄ alkyl)-, —CO—N(C₃-C₆ cycloalkyl)-, —NH—, —N(C₁-C₄ alkyl)-, —N(C₃-C₆ cycloalkyl)-, —CO—, —NH—CO—, —N(C₁-C₄ alkyl)-CO—, —N(C₃-C₆ cycloalkyl)-CO—, —O—, —S—, —CH₂—, —CH(C₁-C₄ alkyl)-, —CH(C₃-C₆ cycloalkyl)-, —C(C₁-C₄ alkyl)(C₁-C₄ alkyl)-, —C(C₁-C₄ alkyl)(C₃-C₆ cycloalkyl)-, —C(C₂-C₆ alkylene)-, —C(C₂-C₆ heteroalkylene)-, —O—CH₂—, —O—CH(C₁-C₄ alkyl)-, —O—CH(C₃-C₆ cycloalkyl)-, —O—C(C₁-C₄ alkyl)(C₁-C₄ alkyl)-, —O—C(C₁-C₄ alkyl)(C₃-C₆ cycloalkyl)-, —O—C(C₂-C₆ alkylene)-, —O—C(C₂-C₆ heteroalkylene)-, —NH—CH₂—, —NH—CH(C₁-C₄ alkyl)-, —NH—CH(C₃-C₆ cycloalkyl)-, —NH—C(C₁-C₄ alkyl)(C₁-C₄ alkyl)-, —NH—C(C₁-C₄ alkyl)(C₃-C₆ cycloalkyl)-, —NH—C(C₂-C₆ alkylene)-, and —NH—C(C₂-C₆ heteroalkylene)-.

In some embodiments, L_{E}¹, L_{E}², L_{E}³ and L_{E}⁴ are divalent groups independently selected from the groups consisting of -L_{E}^{a}-L_{E}^{b}-; wherein L_{E}^{a} and L_{E}^{b}, at each occurrence, are independently selected from null, —CO—, —O—, —S—, —CH₂—, —CH(CH₃)—, —C(CH₃)₂—, —CF₂—, —C(CH₂)₂—, —NH—, —N(CH₃)—, —N(CF₃)—, —N(iPr)-, and —N(cPr)-. In some embodiments, L_{E}¹, L_{E}², L_{E}³ and L_{E}⁴ are divalent groups independently selected from null, —CONH—, —NH—, —O—, —S—, and —CH₂—. In some embodiments, L_{E}¹ is a divalent group selected from null, —CONH—, and —NH—. In some embodiments, Ring A_{E} is of Formulae A_{E}¹, A_{E}², A_{E}³, A_{E}⁴, A_{E}⁵, A_{E}⁶ and A_{E}⁷, and L_{E}¹ is null.

In some embodiments, Ring A_{E} is of Formula A_{E}³, L_{E}¹ is —CONH— or —NH—, and the CRBN modulator is a compound consisting of

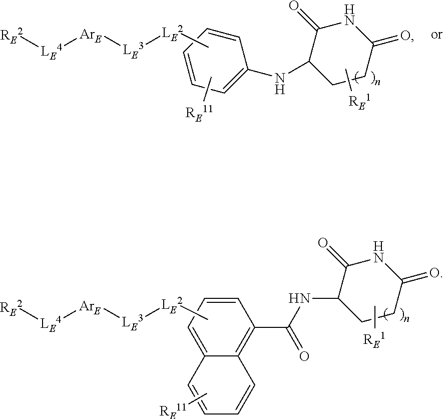

In some embodiments, L_{E}² is a divalent group selected from the group consisting of null, —O—, —NH—, —CH₂—, —S—, and —CONH—. In some embodiments, L_{E}³ is a divalent group selected from null and —CH₂—. In some embodiments, L_{E}⁴ is a divalent group selected from null and —CH₂—. In some embodiments, R_{E}¹ is selected from H, F, Cl, CH₃, CF₃, or CHF₂. In some embodiments, R_{E}¹ is H. In some embodiments, R_{E}² is selected from the group consisting of —R_{E}²b-R_{E}^{2a}; wherein R_{E}²b is null, or selected from the group consisting of optionally substituted C₃-C₁₂ membered cycloalkylene, and optionally substituted 3-12 membered heterocyclylene; and R_{E}^{2a} is selected from hydrogen, halogen, cyano, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl.

In some embodiments, R_{E}² is selected from the group consisting of —R_{E}²b-R_{E}^{2a}; wherein R_{E}²b is null, or selected from the group consisting of optionally substituted C₃-C₁₂ cycloalkylene, optionally substituted 3-12 membered heterocyclylene, optionally substituted aryl, and optionally substituted heteroaryl; and R_{E}^{2a} is selected from optionally substituted C₃-C₁₂ cycloalkyl, optionally substituted 3-12 membered heterocyclyl, optionally substituted aryl, and optionally substituted heteroaryl.

In some embodiments, R_{E}^{2b} is selected from null, or optionally substituted groups consisting of

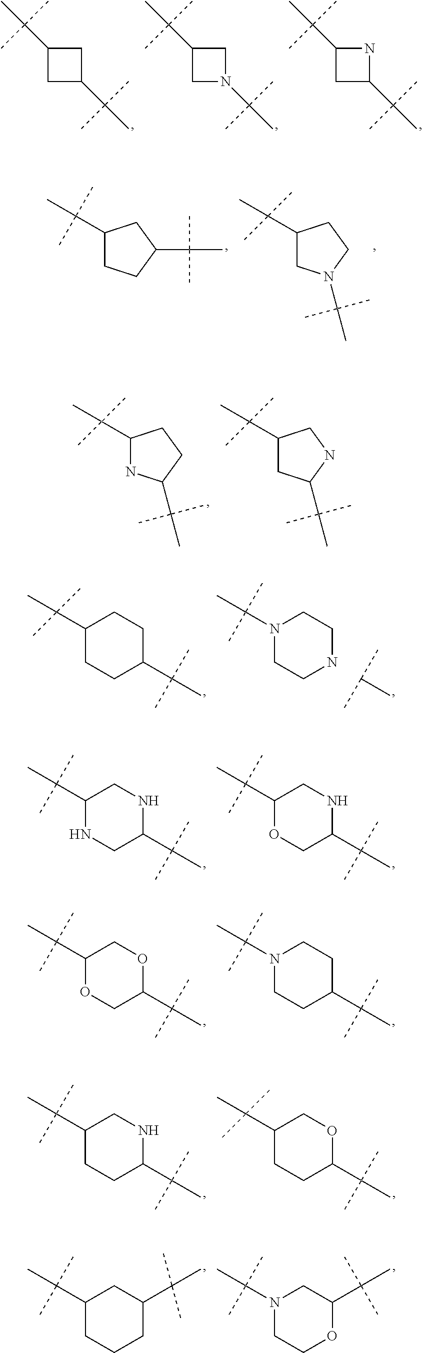

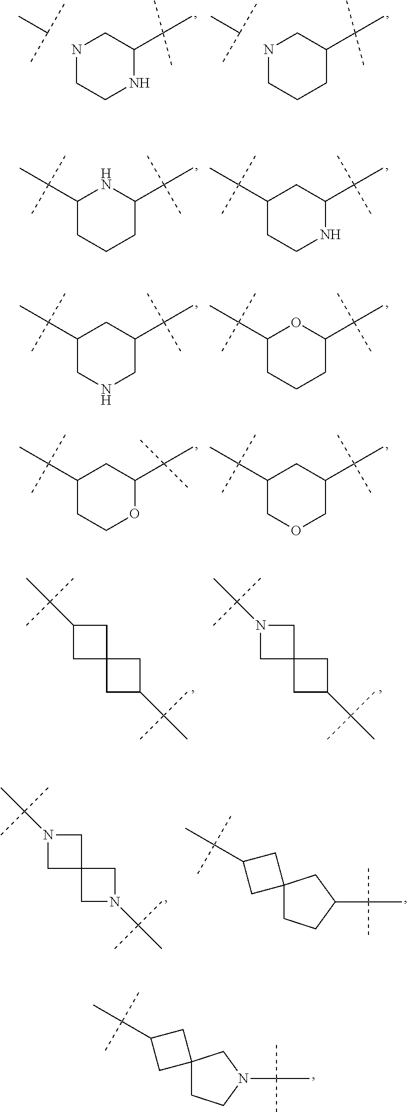

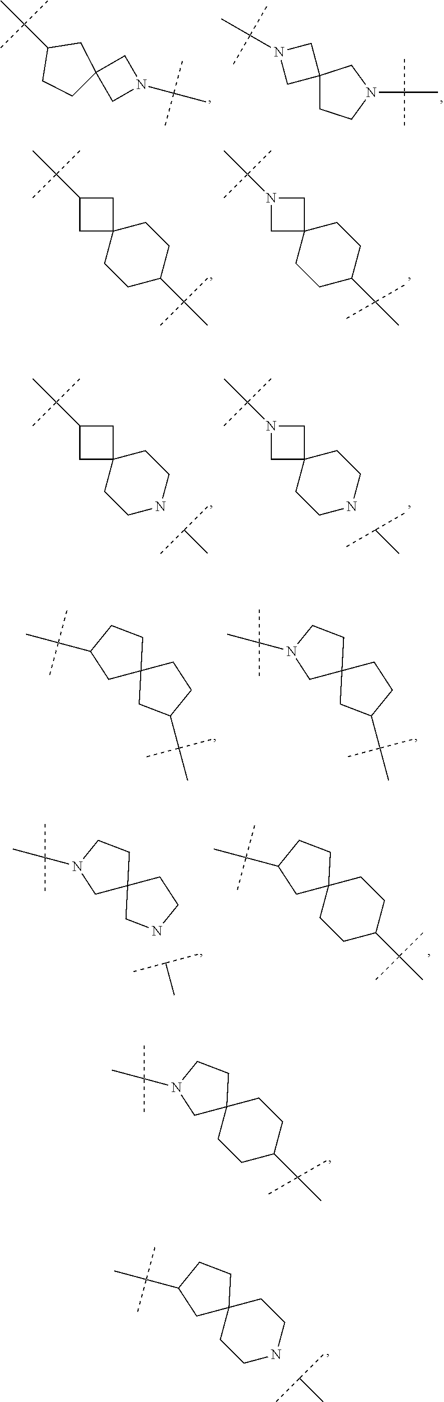

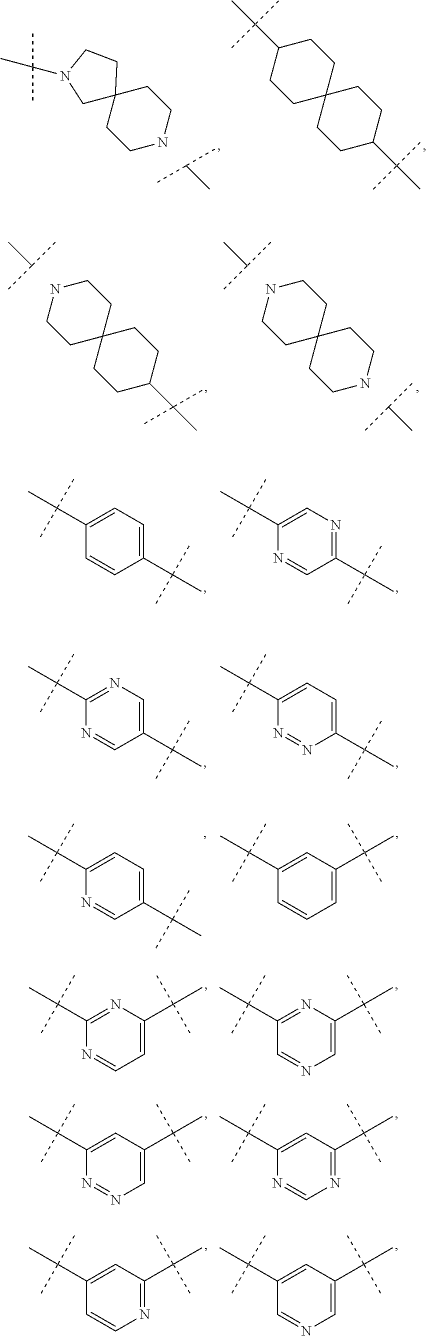

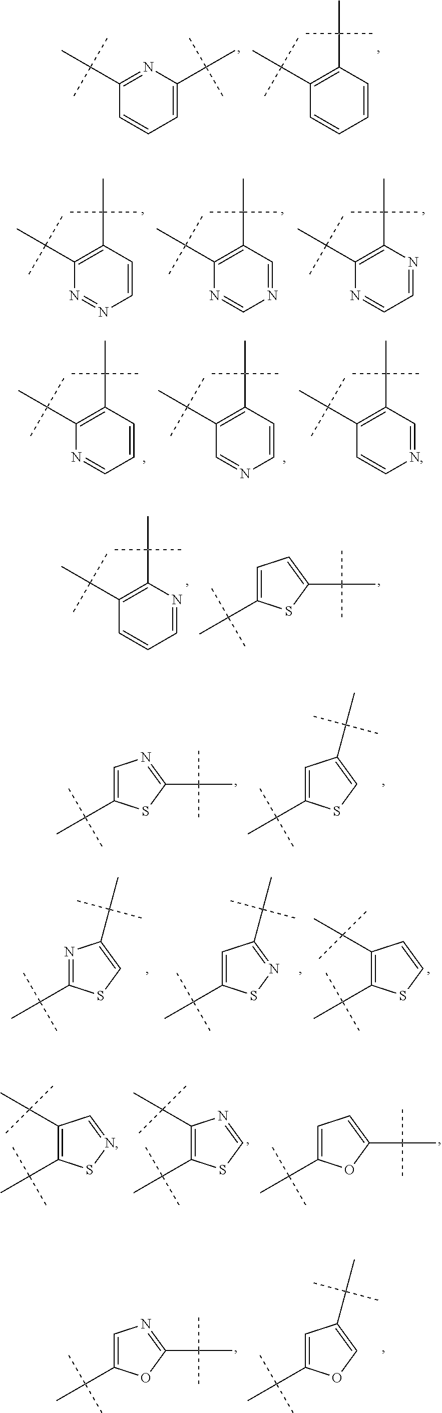

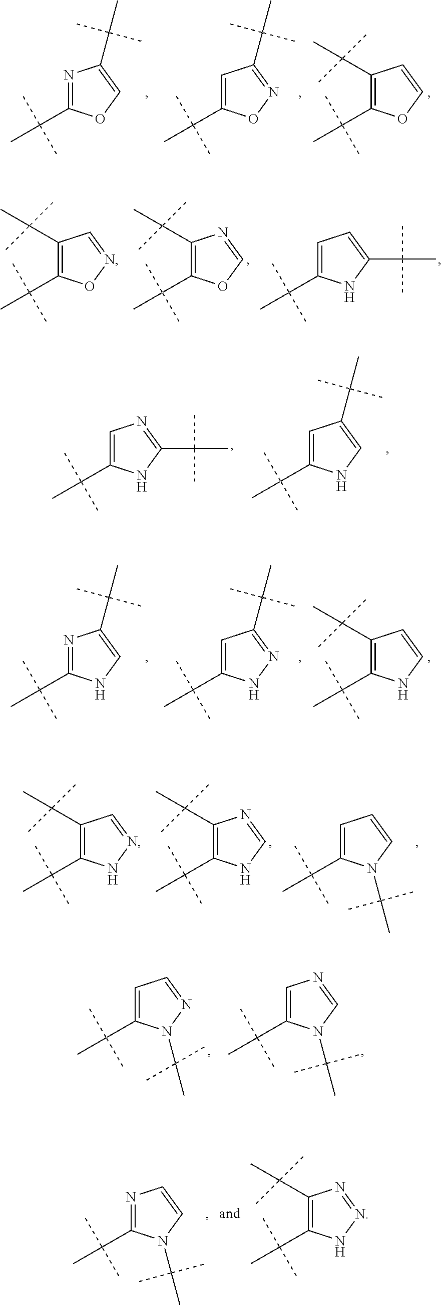

In some embodiments, R_{E}^{2a} is selected from null, hydrogen, halogen, cyano, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, or optionally substituted groups consisting of

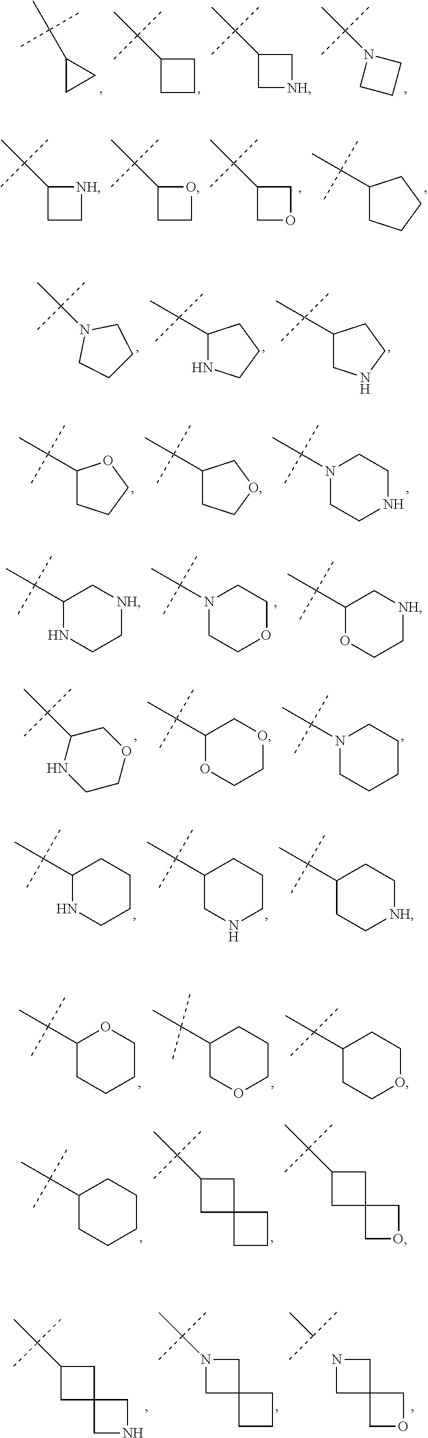

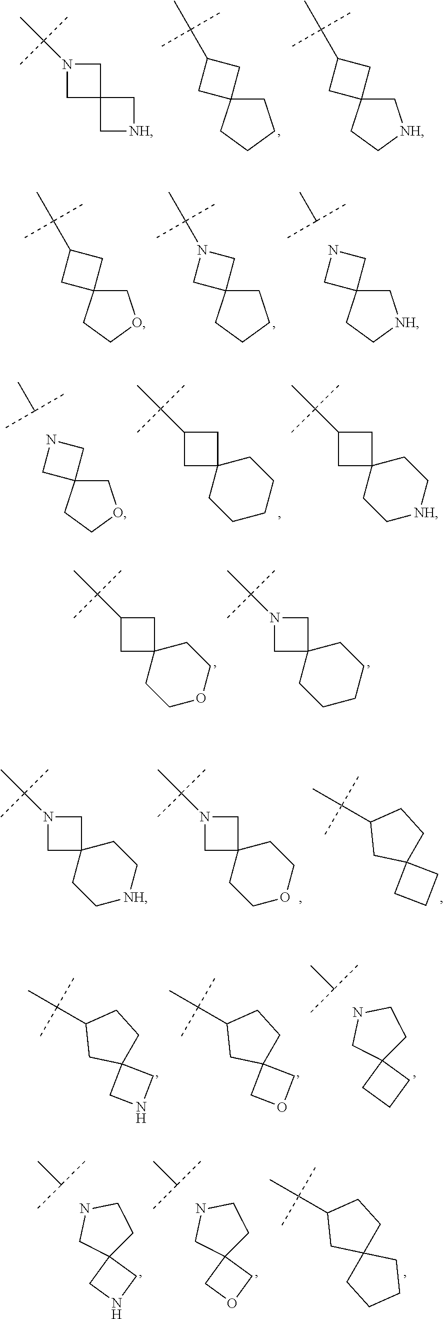

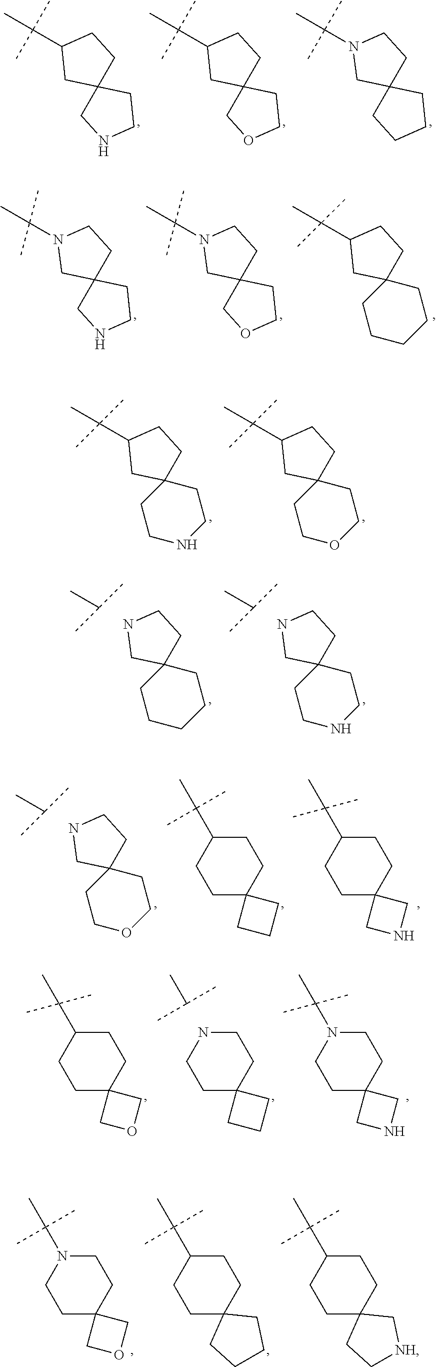

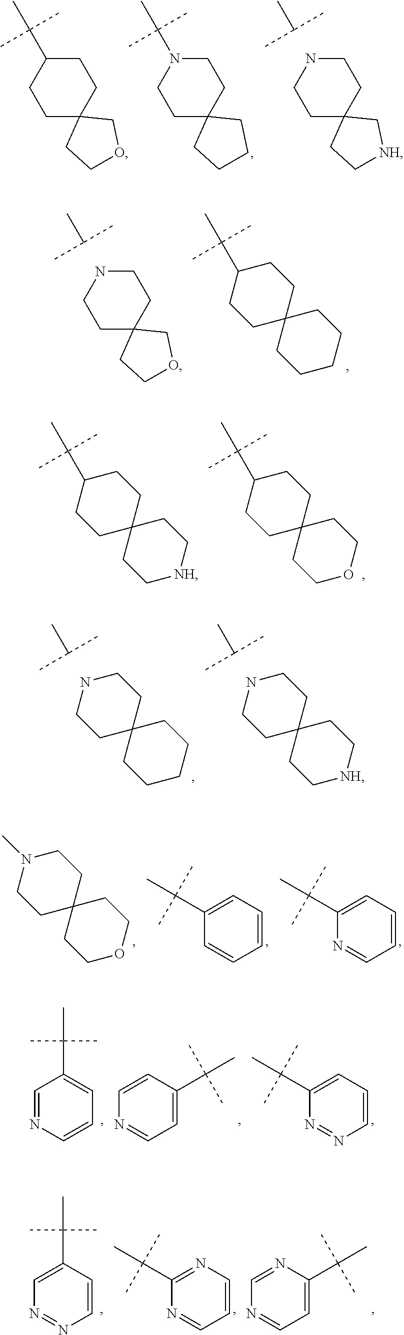

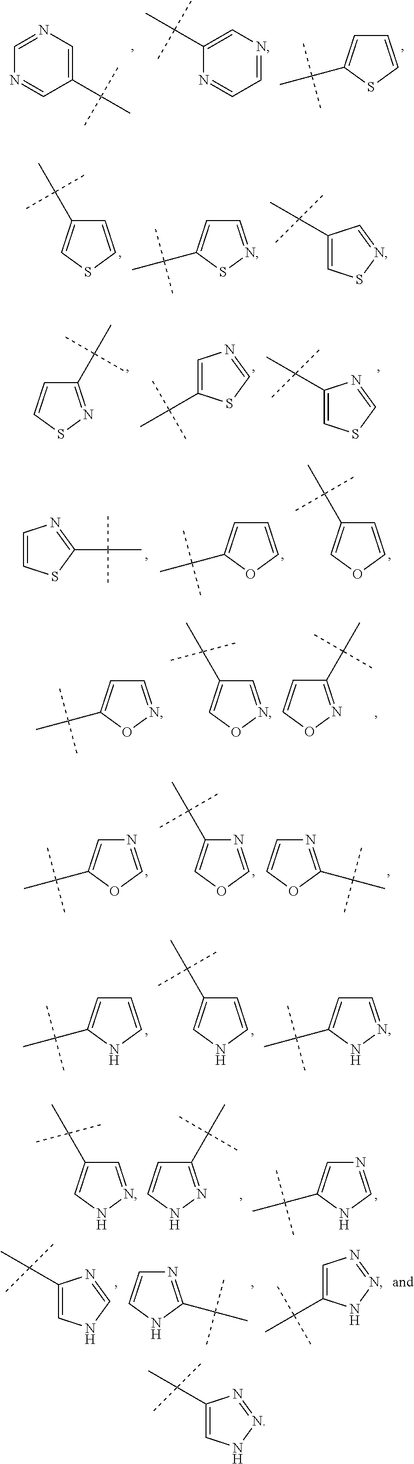

In some embodiments, R_{E}² is selected from

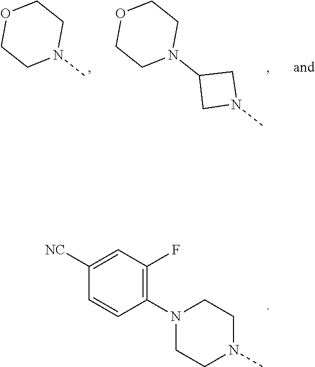

In some embodiments, AR_{E} is selected from optionally substituted groups consisting of

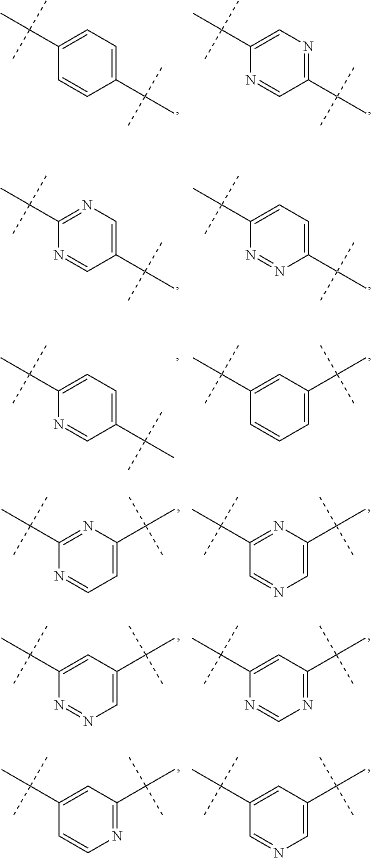

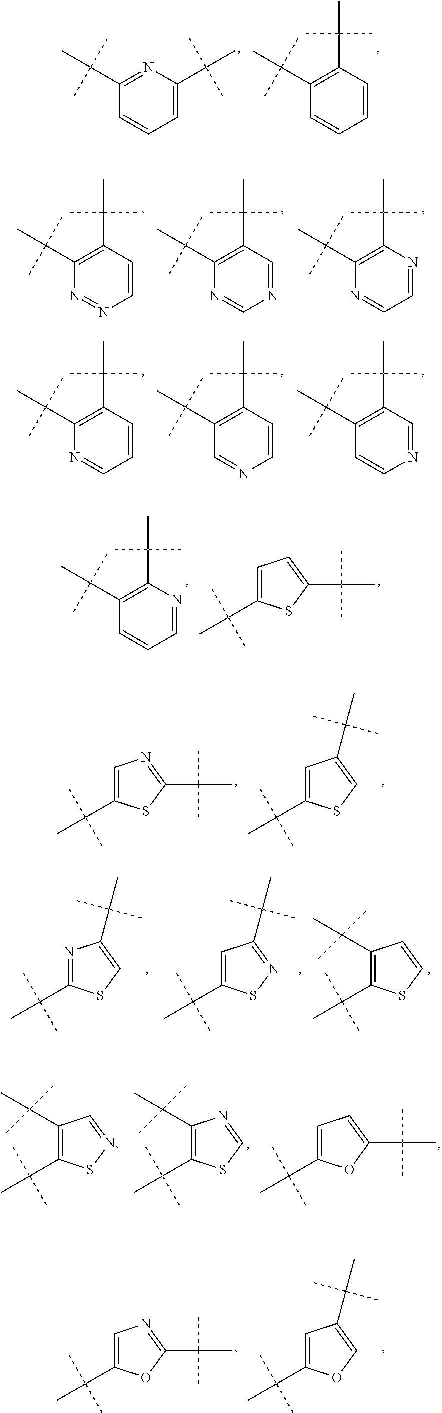

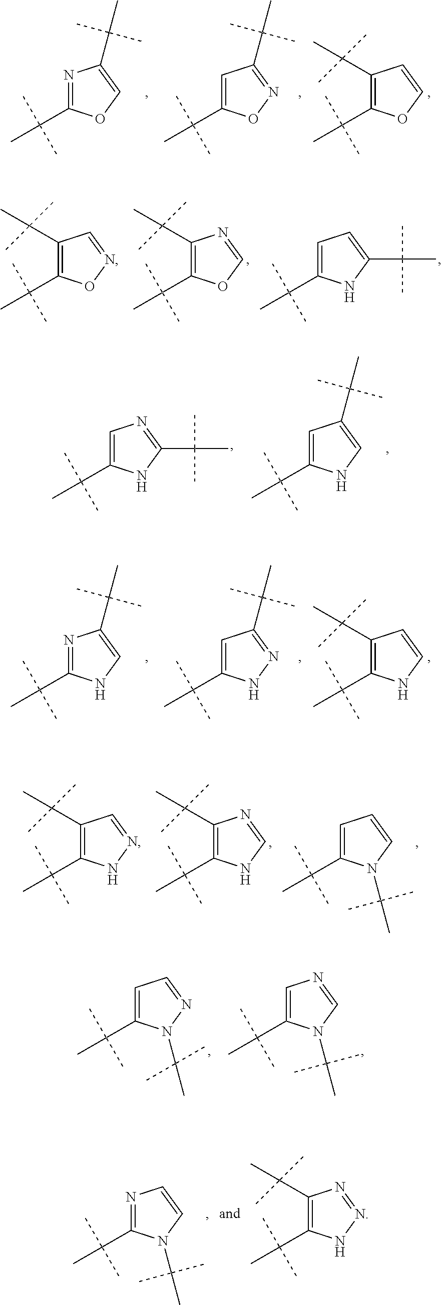

In some embodiments, Ar_{E} is selected from optionally substituted aryl. In some embodiments, Ar_{E} is selected from optionally substituted phenyl. In some embodiments, Ar_{E} is selected from

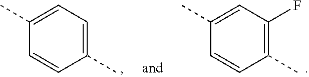

In some embodiments, n is 0 or 1. In some embodiments, n is 1.

In some embodiments, ring A_{E} is Formulae A_{E}¹, and the second compound (e.g., CRBN modulator) is a compound consisting of

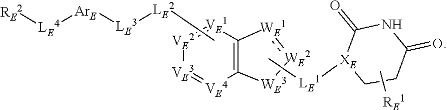

In some embodiments, ring A_{E} is Formulae A_{E}1-a, and the second compound (e.g., CRBN modulator) is a compound consisting of

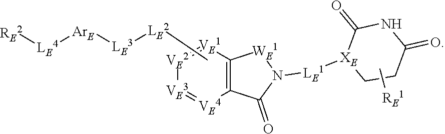

In some embodiments, ring A_{E} is Formulae A_{E}1-a; L_{E}¹ is null; and the second compound (e.g., CRBN modulator) is a compound consisting of

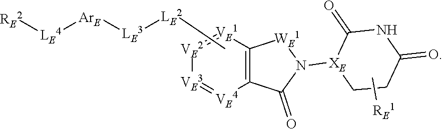

In some embodiments, ring A_{E} is Formulae A_{E}¹-a; L_{E}¹ is null; V_{E}¹, V_{E}², V_{E}³, and V_{E}⁴ are CH; W_{E}¹ is CHR_{E}¹² or CO; and the second compound (e.g. CRBN modulator) is a compound consisting of

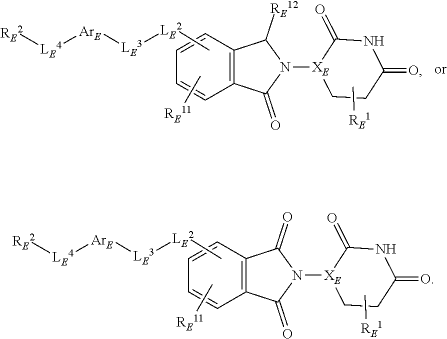

In some embodiments, ring A_{E} is Formulae A_{E}¹-a; L_{E}¹ is null; V_{E}¹, V_{E}², V_{E}³, and V_{E}⁴ are CH; W_{E}¹ is CHR_{E}¹² or CO; X_{E} is CH, and the second compound (e.g. CRBN modulator) is a compound consisting of

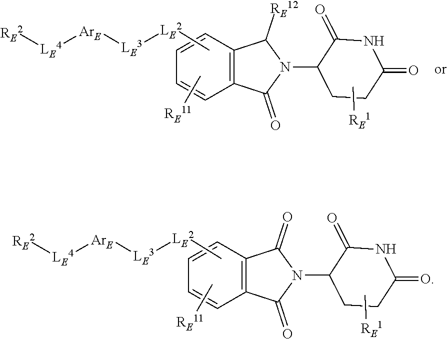

In some embodiments, ring A_{E} is Formulae A_{E}l-a; L_{E}¹ is null; V_{E}¹, V_{E}², V_{E}³, and V_{E}⁴ are CH; W_{E}¹ is CHR_{E}¹² or CO; X_{E} is CH, Ar_{E} is optionally substituted phenyl; and the second compound (e.g., CRBN modulator) is a compound consisting of

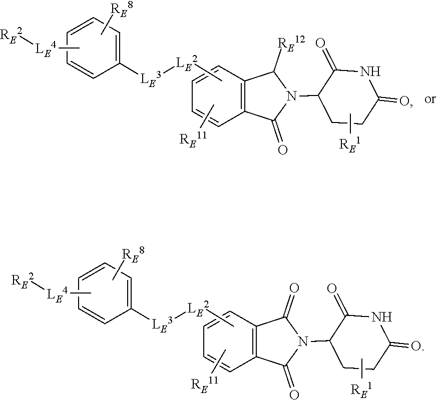

In some embodiments, the second compound is an IMiD, preferably a cereblon modulator. In some embodiments, the cereblon modulator is selected from the group consisting of CC-220 (iberdomide), CC-92480 (mezigdomide), CC-99282 ((S)-2-(2,6-dioxopiperidin-3-yl)-4-((2-fluoro-4-((3-morpholinoazetidin-1-yl)methyl)benzyl)amino)isoindoline-1,3-dione; CAS No.: 2379572-34-4), DKY709 (3-(5-(1-benzylpiperidin-4-yl)-1-oxoisoindolin-2-yl)piperidine-2,6-dione; CAS No.: 2291360-73-9), CFT7455 ((S)-3-(6-(4-(morpholinomethyl)benzyl)-2-oxobenzo[cd]indol-1(2H)-yl)piperidine-2,6-dione; CAS No.: 2504235-67-8), and CC-122 (avadomide), or a pharmaceutically acceptable salt thereof. In some embodiments, the second compound is CC-122, CC-220, or CC-92480. In some embodiments, the second compound is CC-99282 or CFT7455. In some embodiments, the second compound is thalidomide, lenalidomide, or pomalidomide. In some embodiments, the IMiD is an IKZF1 and/or IZKF3 degrader or modulator. In some embodiments, the IMiD is an IKZF1/3 degrader or modulator.

In some embodiments, the second compound (e.g., CRBN modulator) is selected from the group consisting of CC-220, CC-92480, CC-99282, DKY709, CFT7455, and CC-122, or a pharmaceutically acceptable salt thereof. In some embodiments, the second compound is CC-220. In some embodiments, the second compound is CC-92480. In some embodiments, the second compound is CC-99282. In some embodiments, the second compound is DKY709. In some embodiments, the second compound is CFT7455. In some embodiments, the second compound is CC-122.

In some embodiments, the second compound (e.g. CRBN modulator) is a compound consisting of

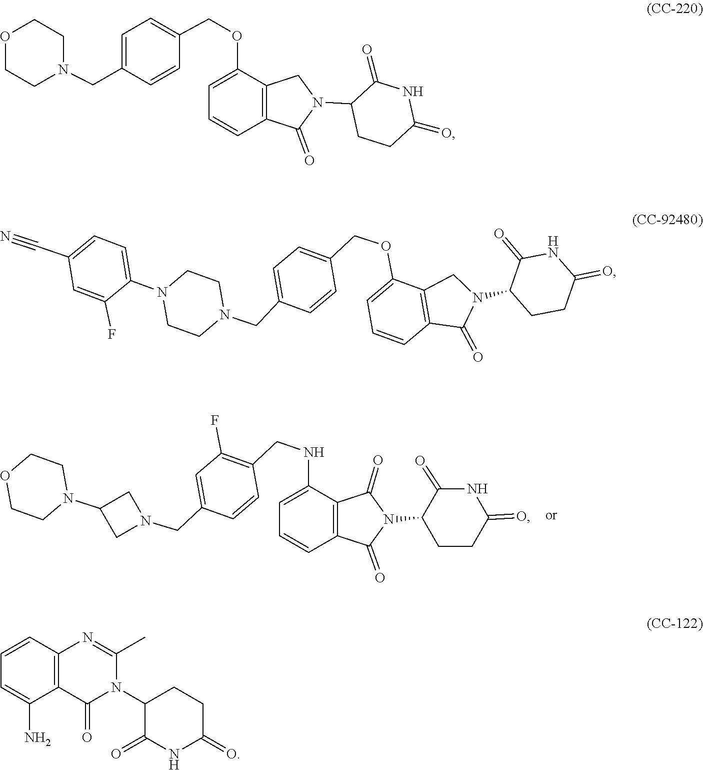 [SMILES: O=C1CCC(N2Cc3c(OCc4ccc(CN5CCOCC5)cc4)cccc3C2=O)C(=O)N1 ; N#Cc1ccc(N2CCN(Cc3ccc(COc4cccc5c4CN([C@H]4CCC(=O)NC4=O)C5=O)cc3)CC2)c(F)c1 ; O=C1CC[C@H](N2C(=O)c3cccc(NCc4ccc(CN5CC(N6CCOCC6)C5)cc4F)c3C2=O)C(=O)N1 ; "or" ; Cc1nc2cccc(N)c2c(=O)n1C1CCC(=O)NC1=O]

In some embodiments, the second compound (e.g., CRBN modulator) comprises thalidomide, lenalidomide, or pomalidomide. In some embodiments, the CRBN modulator is CC-220. In some embodiments, the CRBN modulator is CC-92480. In some embodiments, the CRBN modulator is CC-122. In some embodiments, the CRBN modulator is thalidomide. In some embodiments, the CRBN modulator is lenalidomide. In some embodiments, the CRBN modulator is pomalidomide. In some embodiments, the CRBN modulator is iberdomide. In some embodiments, the second compound excludes thalidomide, lenalidomide, and/or pomalidomide.

In some embodiments, the cereblon modulator is an IKZF1 and/or IZKF3 degrader or modulator. In some embodiments, the cereblon modulator is an IKZF1/3 degrader or modulator. In some embodiments, the IMiD is an IKZF1/3 degrader or modulator.

### Combination of First Compound and Second Compound

The first compound and the second compound may be combined in a method described herein. For example, the first compound and the second compound may both be administered (e.g., coadministered or administered separately) to a subject in a method of treatment. The first compound and the second compound may be combined in a composition such as in a composition for use in a method of treatment. The first compound and the second compound may be used together in a combination.

The combination of two compounds to treat a cancer can be useful in overcoming cancer resistance. In some embodiments, the first compound is an EZH2 inhibitor, and the second compound is an immunomodulatory imide drug (IMiD). In some embodiments, the first compound is an EZH2 inhibitor, and the second compound is a cereblon inhibitor. IMiDs such as cereblon inhibitors may suppress growth of cancerous cells, improve immune system function, such as through T cell or natural killer (NK) cells, or suppress angiogenesis.

In some embodiments, the methods, compositions, and combinations comprise a first compound and a second compound. In some embodiments, the methods, compositions, and combinations comprise an EZH2 inhibitor and a second compound which is an immunomodulatory drug. In some embodiments, the methods, compositions, and combinations comprise an EZH2 inhibitor and an IMiD. In some compositions, the methods, compositions, and combinations comprise and EZH2 inhibitor and a cereblon modulator.

In some embodiments, the methods, compositions, and combinations comprise a first compound, a second compound, and one or more additional cancer treatments. The additional cancer treatment may be any of those disclosed herein. In some embodiments, the methods, compositions, and combinations comprise an EZH2 inhibitor, an IMiD (e.g., a cereblon modulator) and one additional cancer treatment. In some embodiments, the methods, compositions, and combinations comprise an EZH2 inhibitor, a cereblon modulator and one or more additional cancer treatments. In some embodiments, the methods, compositions, and combinations comprise an EZH2 inhibitor and a cereblon modulator and two additional cancer treatments. In some embodiments, the methods, compositions, and combinations comprise an EZH2 inhibitor and a cereblon modulator and three additional cancer treatments. In some embodiments, the methods, compositions, and combinations comprise an EZH2 inhibitor and a cereblon modulator and four additional cancer treatments. In some embodiments, the methods, compositions, and combinations comprise an EZH2 inhibitor and a cereblon modulator and five additional cancer treatments.

In some cases, the first compound is EPZ-6438 (tazemetostat). In some embodiments, the compositions, combinations and methods comprise EPZ-6438 (tazemetostat) and a second compound. In some embodiments, the compositions, combinations and methods comprise EPZ-6438 (tazemetostat) and an IMiD. In some embodiments, the compositions, combinations and methods comprise EPZ-6438 (tazemetostat) and a cereblon modulator. In some such embodiments, the first compound is EPZ-6438 (tazemetostat) and the second compound is a cereblon modulator selected from the group consisting of CC-122, CC-220, CC-92480, CC-99282 and CFT7455.

In some embodiments, the compositions, combinations and methods comprise EPZ-6438 (tazemetostat) and CC-122 (avadomide). In some embodiments, the compositions, combinations and methods comprise EPZ-6438 (tazemetostat) and CC-122 (avadomide) for the treatment of cancer. In some embodiments, the compositions, combinations and methods comprise EPZ-6438 (tazemetostat) and CC-122 (avadomide) for the treatment of lymphoma. In some embodiments, the compositions, combinations and methods comprise EPZ-6438 (tazemetostat) and CC-122 (avadomide) for the treatment of multiple myeloma.

In some embodiments, the compositions, combinations and methods comprise EPZ-6438 (tazemetostat) and CC-220 (iberdomide). In some embodiments, the compositions, combinations and methods comprise EPZ-6438 (tazemetostat) and CC-220 (iberdomide) for the treatment of a cancer. In some embodiments, the compositions, combinations and methods comprise EPZ-6438 (tazemetostat) and CC-220 (iberdomide) for the treatment of lymphoma. In some embodiments, the compositions, combinations and methods comprise EPZ-6438 (tazemetostat) and CC-220 (iberdomide) for the treatment of multiple myeloma.

In some embodiments, the compositions, combinations and methods comprise EPZ-6438 (tazemetostat) and CC-92480. In some embodiments, the compositions, combinations and methods comprise EPZ-6438 (tazemetostat) and CC-92480 for the treatment of cancer. In some embodiments, the compositions, combinations and methods comprise EPZ-6438 (tazemetostat) and CC-92480 for the treatment of lymphoma. In some embodiments, the composition, combinations s and methods comprise EPZ-6438 (tazemetostat) and CC-92480 for the treatment of multiple myeloma.

In some embodiments, the compositions, combinations and methods comprise EPZ-6438 (tazemetostat) and thalidomide. In some embodiments, the compositions, combinations and methods comprise EPZ-6438 (tazemetostat) and thalidomide for the treatment of cancer. In some embodiments, the compositions, combinations and methods comprise EPZ-6438 (tazemetostat) and thalidomide for the treatment of lymphoma. In some embodiments, the compositions, combinations and methods comprise EPZ-6438 (tazemetostat) and thalidomide for the treatment of multiple myeloma.

In some embodiments, the compositions, combinations and methods comprise EPZ-6438 (tazemetostat) and lenalidomide. In some embodiments, the compositions, combinations and methods comprise EPZ-6438 (tazemetostat) and lenalidomide for the treatment of cancer. In some embodiments, the compositions, combinations and methods comprise EPZ-6438 (tazemetostat) and lenalidomide for the treatment of lymphoma. In some embodiments, the compositions, combinations and methods comprise EPZ-6438 (tazemetostat) and lenalidomide for the treatment of multiple myeloma.

In some embodiments, the compositions, combinations and methods comprise EPZ-6438 (tazemetostat) and pomalidomide. In some embodiments, the compositions, combinations and methods comprise EPZ-6438 (tazemetostat) and pomalidomide for the treatment of cancer. In some embodiments, the compositions, combinations and methods comprise EPZ-6438 (tazemetostat) and pomalidomide for the treatment of lymphoma. In some embodiments, the compositions, combinations and methods comprise EPZ-6438 (tazemetostat) and pomalidomide for the treatment of multiple myeloma.

In some cases, the first compound is GSK126. In some embodiments, the compositions, combinations and methods comprise GSK126 and a second compound. In some embodiments, the compositions, combinations and methods comprise GSK126 and an IMiD. In some embodiments, the compositions, combinations and methods comprise GSK126 and a cereblon modulator. In some such embodiments, the first compound is GSK126, and the second compound is a cereblon modulator selected from the group consisting of CC-122, CC-220, CC-92480, CC-99282 and CFT7455.

In some embodiments, the compositions, combinations and methods comprise GSK126 and CC-122 (avadomide). In some embodiments, the compositions, combinations and methods comprise GSK126 and CC-122 (avadomide) for the treatment of cancer. In some embodiments, the compositions, combinations and methods comprise GSK126 and CC-122 (avadomide) for the treatment of lymphoma. In some embodiments, the compositions, combinations and methods comprise GSK126 and CC-122 (avadomide) for the treatment of multiple myeloma.

In some embodiments, the compositions, combinations and methods comprise GSK126 and CC-220 (iberdomide). In some embodiments, the compositions, combinations and methods comprise GSK126 and CC-220 (iberdomide) for the treatment of a cancer. In some embodiments, the compositions, combinations and methods comprise GSK126 and CC-220 (iberdomide) for the treatment of lymphoma. In some embodiments, the compositions, combinations and methods comprise GSK126 and CC-220 (iberdomide) for the treatment of multiple myeloma.

In some embodiments, the compositions, combinations and methods comprise GSK126 and CC-92480. In some embodiments, the compositions, combinations and methods comprise GSK126 and CC-92480 for the treatment of cancer. In some embodiments, the compositions, combinations and methods comprise GSK126 and CC-92480 for the treatment of lymphoma. In some embodiments, the compositions, combinations and methods comprise GSK126 and CC-92480 for the treatment of multiple myeloma.

In some cases, the first compound is CPI-1205 (lirametostat). In some embodiments, the compositions, combinations and methods comprise is CPI-1205 (lirametostat) and a second compound. In some embodiments, the compositions, combinations and methods comprise is CPI-1205 (lirametostat) and an IMiD. In some embodiments, the compositions, combinations and methods comprise is CPI-1205 (lirametostat) and a cereblon modulator. In some such embodiments, the first compound is CPI-1205 (lirametostat) and the second compound is a cereblon modulator selected from the group consisting of CC-122, CC-220, CC-92480, CC-99282 and CFT7455.

In some embodiments, the compositions, combinations and methods comprise CPI-1205 (lirametostat) and CC-122 (avadomide). In some embodiments, the compositions, combinations and methods comprise CPI-1205 (lirametostat) and CC-122 (avadomide) for the treatment of a cancer. In some embodiments, the compositions, combinations and methods comprise CPI-1205 (lirametostat) and CC-122 (avadomide) for the treatment of lymphoma. In some embodiments, the compositions, combinations and methods comprise CPI-1205 (lirametostat) and CC-122 (avadomide) for the treatment of multiple myeloma.

In some embodiments, the compositions, combinations and methods comprise CPI-1205 (lirametostat) and CC-220 (iberdomide). In some embodiments, the compositions, combinations and methods comprise CPI-1205 (lirametostat) and CC-220 (iberdomide) for the treatment of cancer. In some embodiments, the compositions, combinations and methods comprise CPI-1205 (lirametostat) and CC-220 (iberdomide) for the treatment of lymphoma. In some embodiments, the compositions, combinations and methods comprise CPI-1205 (lirametostat) and CC-220 (iberdomide) for the treatment of multiple myeloma.

In some embodiments, the compositions, combinations and methods comprise CPI-1205 (lirametostat) and CC-92480. In some embodiments, the compositions, combinations and methods comprise CPI-1205 (lirametostat) and CC-92480 for the treatment of cancer. In some embodiments, the compositions, combinations and methods comprise CPI-1205 (lirametostat) and CC-92480 for the treatment of lymphoma. In some embodiments, the compositions, combinations and methods comprise CPI-1205 (lirametostat) and CC-92480 for the treatment of multiple myeloma.

In some embodiments, the compositions, combinations and methods comprise CPI-1205 (lirametostat) and thalidomide. In some embodiments, the compositions, combinations and methods comprise CPI-1205 (lirametostat) and thalidomide for the treatment of cancer. In some embodiments, the compositions, combinations and methods comprise CPI-1205 (lirametostat) and thalidomide for the treatment of lymphoma. In some embodiments, the compositions, combinations and methods comprise CPI-1205 (lirametostat) and thalidomide for the treatment of multiple myeloma.

In some embodiments, the compositions, combinations and methods comprise CPI-1205 (lirametostat) and lenalidomide. In some embodiments, the compositions, combinations and methods comprise CPI-1205 (lirametostat) and lenalidomide for the treatment of cancer. In some embodiments, the compositions, combinations and methods comprise CPI-1205 (lirametostat) and lenalidomide for the treatment of lymphoma. In some embodiments, the compositions, combinations and methods comprise CPI-1205 (lirametostat) and lenalidomide for the treatment of multiple myeloma.

In some embodiments, the compositions, combinations and methods comprise CPI-1205 (lirametostat) and pomalidomide. In some embodiments, the compositions, combinations and methods comprise CPI-1205 (lirametostat) and pomalidomide for the treatment of cancer. In some embodiments, the compositions, combinations and methods comprise CPI-1205 (lirametostat) and pomalidomide for the treatment of lymphoma. In some embodiments, the compositions, combinations and methods comprise CPI-1205 (lirametostat) and pomalidomide for the treatment of multiple myeloma.

In some embodiments, the first compound is C24. In some embodiments, the compositions and method comprise C24 and a second compound. In some embodiments, the compositions, combinations and methods comprise is C24 and an IMiD. In some embodiments, the compositions, combinations and methods comprise is C24 and a cereblon modulator. In some such embodiments, the first compound is C24 and the second compound is a cereblon modulator selected from the group consisting of CC-122, CC-220, CC-92480, CC-99282 and CFT7455.

In some embodiments, the compositions, combinations and methods comprise C24 and CC-122 (avadomide). In some embodiments, the compositions, combinations and methods comprise C24 and CC-122 (avadomide) for the treatment of cancer. In some embodiments, the compositions, combinations and methods comprise C24 and CC-122 (avadomide) for the treatment of lymphoma. In some embodiments, the compositions, combinations and methods comprise C24 and CC-122 (avadomide) for the treatment of multiple myeloma.

In some embodiments, the compositions, combinations and methods comprise C24 and CC-220 (iberdomide). In some embodiments, the compositions, combinations and methods comprise C24 and CC-220 (iberdomide) for the treatment of cancer. In some embodiments, the compositions, combinations and methods comprise C24 and CC-220 (iberdomide) for the treatment of lymphoma. In some embodiments, the compositions, combinations and methods comprise C24 and CC-220 (iberdomide) for the treatment of multiple myeloma.

In some embodiments, the compositions, combinations and methods comprise C24 and CC-92480. In some embodiments, the compositions, combinations and methods comprise C24 and CC-92480 for the treatment of cancer. In some embodiments, the compositions, combinations and methods comprise C24 and CC-92480 for the treatment of lymphoma. In some embodiments, the compositions, combinations and methods comprise C24 and CC-92480 for the treatment of multiple myeloma.

In some embodiments, the compositions, combinations and methods comprise C24 and thalidomide. In some embodiments, the compositions, combinations and methods comprise C24 and thalidomide for the treatment of cancer. In some embodiments, the compositions, combinations and methods comprise C24 and thalidomide for the treatment of lymphoma. In some embodiments, the compositions, combinations and methods comprise C24 and thalidomide for the treatment of multiple myeloma.

In some embodiments, the compositions, combinations and methods comprise C24 and lenalidomide. In some embodiments, the compositions, combinations and methods comprise C24 and lenalidomide for the treatment of cancer. In some embodiments, the compositions, combinations and methods comprise C24 and lenalidomide for the treatment of lymphoma. In some embodiments, the compositions, combinations and methods comprise C24 and lenalidomide for the treatment of multiple myeloma.

In some embodiments, the compositions, combinations and methods comprise C24 and pomalidomide. In some embodiments, the compositions, combinations and methods comprise C24 and pomalidomide for the treatment of cancer. In some embodiments, the compositions, combinations and methods comprise C24 and pomalidomide for the treatment of lymphoma. In some embodiments, the compositions, combinations and methods comprise C24 and pomalidomide for the treatment of multiple myeloma.

In some cases, the first compound is CPI-0209. In some embodiments, the compositions, combinations and methods comprise CPI-0209 and a second compound. In some embodiments, the compositions, combinations and methods comprise CPI-0209 and an IMiD. In some embodiments, the compositions, combinations and methods comprise CPI-0209 and a cereblon modulator. In some such embodiments, the first compound is CPI-0209 and the second compound is a cereblon modulator selected from the group consisting of CC-122, CC-220, CC-92480, CC-99282 and CFT7455.

In some embodiments, the compositions, combinations and methods comprise CPI-0209 and CC-99282. In some embodiments, the compositions, combinations and methods comprise CPI-0209 and CC-99282 for the treatment of cancer. In some embodiments, the compositions, combinations and methods comprise CPI-0209 and CC-99282 for the treatment of lymphoma. In some embodiments, the compositions, combinations and methods comprise CPI-0209 and CC-99282 for the treatment of multiple myeloma.

In some embodiments, the compositions, combinations and methods comprise CPI-0209 and CFT7455. In some embodiments, the compositions, combinations and methods comprise CPI-0209 and CFT7455 for the treatment of a cancer. In some embodiments, the compositions, combinations and methods comprise CPI-0209 and CFT7455 for the treatment of lymphoma. In some embodiments, the compositions, combinations and methods comprise CPI-0209 and CFT7455 for the treatment of multiple myeloma.

In some embodiments, the compositions, combinations and methods comprise CPI-0209 and thalidomide. In some embodiments, the compositions, combinations and methods comprise CPI-0209 and thalidomide for the treatment of cancer. In some embodiments, the compositions, combinations and methods comprise CPI-0209 and thalidomide for the treatment of lymphoma. In some embodiments, the compositions, combinations and methods comprise CPI-0209 and thalidomide for the treatment of multiple myeloma.

In some embodiments, the compositions, combinations and methods comprise CPI-0209 and lenalidomide. In some embodiments, the compositions, combinations and methods comprise CPI-0209 and lenalidomide for the treatment of cancer. In some embodiments, the compositions, combinations and methods comprise CPI-0209 and lenalidomide for the treatment of lymphoma. In some embodiments, the compositions, combinations and methods comprise CPI-0209 and lenalidomide for the treatment of multiple myeloma.

In some embodiments, the compositions, combinations and methods comprise CPI-0209 and pomalidomide. In some embodiments, the compositions, combinations and methods comprise CPI-0209 and pomalidomide for the treatment of cancer. In some embodiments, the compositions, combinations and methods comprise CPI-0209 and pomalidomide for the treatment of lymphoma. In some embodiments, the compositions, combinations and methods comprise CPI-0209 and pomalidomide for the treatment of multiple myeloma.

In some cases, the first compound is SHR2554. In some embodiments, the compositions, combinations and methods comprise SHR2554 and a second compound. In some embodiments, the compositions, combinations and methods comprise SHR2554 and an IMiD. In some embodiments, the compositions, combinations and methods comprise SHR2554 and a cereblon modulator. In some such embodiments, the first compound is SHR2554, and the second compound is a cereblon modulator selected from the group consisting of CC-122, CC-220, CC-92480, CC-99282 and CFT7455.

In some embodiments, the compositions, combinations and methods comprise SHR2554 and CC-99282. In some embodiments, the compositions, combinations and methods comprise SHR2554 and CC-99282 for the treatment of cancer. In some embodiments, the compositions, combinations and methods comprise SHR2554 and CC-99282 for the treatment of lymphoma. In some embodiments, the compositions, combinations and methods comprise SHR2554 and CC-99282 for the treatment of multiple myeloma.

In some embodiments, the compositions, combinations and methods comprise SHR2554 and CFT7455. In some embodiments, the compositions, combinations and methods comprise SHR2554 and CFT7455 for the treatment of a cancer. In some embodiments, the compositions, combinations and methods comprise SHR2554 and CFT7455 for the treatment of lymphoma. In some embodiments, the compositions, combinations and methods comprise SHR2554 and CFT7455 for the treatment of multiple myeloma.

In some embodiments, the compositions, combinations and methods comprise SHR2554 and thalidomide. In some embodiments, the compositions, combinations and methods comprise SHR2554 and thalidomide for the treatment of cancer. In some embodiments, the compositions, combinations and methods comprise SHR2554 and thalidomide for the treatment of lymphoma. In some embodiments, the compositions, combinations and methods comprise SHR2554 and thalidomide for the treatment of multiple myeloma.

In some embodiments, the compositions, combinations and methods comprise SHR2554 and lenalidomide. In some embodiments, the compositions, combinations and methods comprise SHR2554 and lenalidomide for the treatment of cancer. In some embodiments, the compositions, combinations and methods comprise SHR2554 and lenalidomide for the treatment of lymphoma. In some embodiments, the compositions, combinations and methods comprise SHR2554 and lenalidomide for the treatment of multiple myeloma.

In some embodiments, the compositions, combinations and methods comprise SHR2554 and pomalidomide. In some embodiments, the compositions, combinations and methods comprise SHR2554 and pomalidomide for the treatment of cancer. In some embodiments, the compositions, combinations and methods comprise SHR2554 and pomalidomide for the treatment of lymphoma. In some embodiments, the compositions, combinations and methods comprise SHR2554 and pomalidomide for the treatment of multiple myeloma.

### Pharmaceutical Compositions

Disclosed herein, in some embodiments, are pharmaceutical compositions comprising a first compound or a second compound. A first pharmaceutical composition may include the first compound. A second pharmaceutical composition may include the second compound. Disclosed herein, in some embodiments, are pharmaceutical compositions comprising a first compound and a second compound. For example, the pharmaceutical composition may include both the first compound and the second compound. In some embodiments, the pharmaceutical composition comprises one or more additional pharmaceutical cancer treatments. In some embodiments, any of the compounds described herein are combined with a pharmaceutically acceptable carrier. The pharmaceutically acceptable carrier may also be referred to herein as a pharmaceutically acceptable excipient, physiologically acceptable excipient, or physiologically acceptable carrier. The pharmaceutically acceptable carrier may be selected on the basis of a chosen route of administration.

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### Methods of Treatment

Disclosed herein, in some embodiments, are methods of treating a cancer. The method can include administering any of the compositions described herein. In some embodiments, treating a cancer comprises administering the composition to a subject in need thereof. In some embodiments, the method comprises administering the composition to a subject comprising a cancer. Administration of the first compound before, after, or at the same time as the second compound. In some embodiments, the EZH2 inhibitor is administered before the IMiD. In some embodiments, the EZH2 inhibitor is administered with the IMiD. In some embodiments, the EZH2 inhibitor is administered after the IMiD. In some embodiments, the EZH2 inhibitor is administered before the cereblon modulator. In some embodiments, the EZH2 inhibitor is administered with the cereblon modulator. In some embodiments, the EZH2 inhibitor is administered after the cereblon modulator.

Disclosed herein, in certain embodiments, are compositions, combinations and methods for treating cancers. The treatment comprises administering to a subject in need of a cancer treatment, a first compound and administering to the subject a second compound. In some embodiments, the first compound is an Enhancer of Zeste Homolog 2 (EZH2) inhibitor. The EZH2 inhibitor may comprise one or more EZH2 inhibitors. In some embodiments, the second compound comprises an immunomodulatory drug. The first compound may be administered before the second compound, at the same time as the second compound, or after the second compound.

The compositions, combinations and methods disclosed herein may be useful in the treatment of cancer. In some embodiments, the cancer is a blood cancer. In some embodiments, the blood cancer comprises a lymphoma, leukemia, or multiple myeloma. In some embodiments, the blood cancer comprises lymphoma. In some embodiments, the blood cancer comprises leukemia. In some embodiments, the blood cancer comprises multiple myeloma.

In some embodiments, the compositions, combinations and methods are used to treat a blood cancer. Lymphomas, leukemias and multiple myelomas may each demonstrate different symptom presentation, prognoses, and test/imaging results. For example, lymphomas involve lymphocytes, whereas myelomas involve plasma cells that make an abnormal protein. The abnormal protein is known by different names, such as monoclonal immunoglobulin, monoclonal protein (M-protein), M-spike, or paraprotein. Due to the complexity of these cancers, treatment approaches may vary when comparing one cancer to another cancer. While treatment approaches vary based on multiple factors, such as the type and stage of cancer, patient comorbidities, age, etc., the general first line treatments often differ depending on the cancer being treated. For example, diffuse large B-cell lymphoma may be treated with cyclophosphamide, doxorubicin, vincristine, or prednisone plus rituximab, whereas for multiple myeloma, induction therapy for transplant eligible patients may include bortezomib, lenalidomide or dexamethasone.

In some embodiments, the blood cancer comprises lymphoma. In some embodiments, the lymphoma comprises Hodgkin's lymphoma. In some embodiments, the lymphoma comprises Non-Hodgkin's lymphoma. In some embodiments, the Non-Hodgkin's lymphoma comprises Burkitt lymphoma. In some embodiments, the Non-Hodgkin's lymphoma comprises diffuse large B-cell lymphoma (DLBCL). In some embodiments, the Non-Hodgkin's lymphoma comprises lymphoblastic lymphoma. In some embodiments, the Non-Hodgkin's lymphoma comprises anaplastic large cell lymphoma. In some embodiments, the Non-Hodgkin's lymphoma comprises mantle cell lymphoma. In some embodiments, the Non-Hodgkin's lymphoma comprises follicular lymphoma. In some embodiments, the Non-Hodgkin's lymphoma comprises a mucosa-associated lymphoid tissue (MALT) lymphoma. In some embodiments, the Non-Hodgkin's lymphoma comprises a marginal zone lymphoma. In some embodiments, the Non-Hodgkin's lymphoma comprises primary central nervous system lymphoma. In some embodiments, the Non-Hodgkin's lymphoma comprises peripheral T-cell lymphoma. In some embodiments, the Non-Hodgkin's lymphoma comprises acute lymphoblastic leukemia/lymphoma. In some embodiments, the Non-Hodgkin's lymphoma comprises adult T-cell leukemia-lymphoma. In some embodiments, the Non-Hodgkin's lymphoma comprises small lymphocytic lymphoma. In some embodiments, the Non-Hodgkin's lymphoma comprises a primary cutaneous B-cell lymphoma. In some embodiments, the Non-Hodgkin's lymphoma comprises Mycosis fungoides/Sezary syndrome. In some embodiments, the Non-Hodgkin's lymphoma comprises hairy cell leukemia. In some embodiments, the Non-Hodgkin's lymphoma comprises Waldenstrom Macroglobulinemia. In some embodiments, the Non-Hodgkin's lymphoma is post-transplant lymphoproliferative disorder. In some embodiments, the non-Hodgkin's lymphoma comprises follicular lymphoma or a diffuse large B-cell lymphoma.

In some embodiments, the subject in need of a cancer treatment comprises a cancer. In some embodiments, the subject comprises stage 1 lymphoma, stage 2 lymphoma, stage 3 lymphoma, or stage 4 lymphoma. Stage 1 lymphoma is present when lymphoma is present one group of lymph nodes or one organ. Stage 2 lymphoma is present when two or more lymph nodes are affected, and the cancer is either above or below the diaphragm. Stage 3 lymphoma is present when the lymphoma is present both above and below the diaphragm. Stage 4 lymphoma is present when outside of the lymphatic system. The lymphatic system comprises the spleen and the thymus. In some embodiments, the treatment decreases the stage of the cancer from stage 4 to stage 3, stage 4 to stage 2, stage 4 to stage 1, stage 3 to stage 2, stage 3 to stage 1, or stage 2 to stage 1.

In some embodiments, the compositions, combinations and methods reduce the cancer's ability to metastasize. In some embodiments, the compositions, combinations and methods reduce the size or amount of a secondary tumor. In some embodiments, the compositions, combinations and methods reduce the size or amount of a primary tumor.

In some embodiments, the compositions, combinations and methods are administered to a subject demonstrating signs and/or symptoms of lymphoma. In some embodiments, the compositions, combinations and methods are administered to a subject suspected of having lymphoma. In some embodiments, the compositions, combinations and methods are administered to a subject comprising lymphoma.

In some embodiments, the disclosed treatment increases the survival rate of a population of subjects. In some cases, the survival rate is measured as the 5-year survival rate. In some embodiments, the survival rate is increased as compared to a population not receiving a treatment. In some embodiments, the survival rate is increased as compared to a population receiving a different treatment regimen. In some embodiments, the survival rate is increased by 1%, 2%, 3%, 4%, 5%, 6%, 7%, 8%, 9%, 10%, 11% 12%, 13%, 14% 15%, or 20%. In some embodiments, the survival rate is increased by at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, or at least 20%.

In some embodiments, the composition or method reduces cancer cell viability. In some embodiments, an EZH2 inhibitor reduces cancer cell viability. In some embodiments, an IMiD reduces cancer cell viability. In some embodiments, a cereblon modulator reduces cancer cell viability. In some embodiments, the first and second compounds reduce cancer cell viability. In some embodiments, the first and second compounds decrease viability of the cancer cells in the subject. In some embodiments, the first and second compounds decrease viability of the cancer cells when removed from the subject. Cancer cell viability may include the cell's ability to survive, live successfully (i.e., survive), ability to reproduce, ability to spread (i.e., metastasize), or ability to grow. In some embodiments, the first or second compound reduces cancer cell viability by at least 10%, at least 25%, or at least 50%. In some embodiments, the first or second compound reduces cancer cell viability by at least about 10% to about 100%. In some embodiments, the first or second compound reduces cancer cell viability by at least about 10% to about 20%, about 10% to about 25%, about 10% to about 30%, about 10% to about 40%, about 10% to about 50%, about 10% to about 60%, about 10% to about 70%, about 10% to about 75%, about 10% to about 80%, about 10% to about 90%, about 10% to about 100%, about 20% to about 25%, about 20% to about 30%, about 20% to about 40%, about 20% to about 50%, about 20% to about 60%, about 20% to about 70%, about 20% to about 75%, about 20% to about 80%, about 20% to about 90%, about 20% to about 100%, about 25% to about 30%, about 25% to about 40%, about 25% to about 50%, about 25% to about 60%, about 25% to about 70%, about 25% to about 75%, about 25% to about 80%, about 25% to about 90%, about 25% to about 100%, about 30% to about 40%, about 30% to about 50%, about 30% to about 60%, about 30% to about 70%, about 30% to about 75%, about 30% to about 80%, about 30% to about 90%, about 30% to about 100%, about 40% to about 50%, about 40% to about 60%, about 40% to about 70%, about 40% to about 75%, about 40% to about 80%, about 40% to about 90%, about 40% to about 100%, about 50% to about 60%, about 50% to about 70%, about 50% to about 75%, about 50% to about 80%, about 50% to about 90%, about 50% to about 100%, about 60% to about 70%, about 60% to about 75%, about 60% to about 80%, about 60% to about 90%, about 60% to about 100%, about 70% to about 75%, about 70% to about 80%, about 70% to about 90%, about 70% to about 100%, about 75% to about 80%, about 75% to about 90%, about 75% to about 100%, about 80% to about 90%, about 80% to about 100%, or about 90% to about 100%. In some embodiments, the first or second compound reduces cancer cell viability by at least about 10%, about 20%, about 25%, about 30%, about 40%, about 50%, about 60%, about 70%, about 75%, about 80%, about 90%, or about 100%. In some embodiments, the first or second compound reduces cancer cell viability by at least at least about 10%, about 20%, about 25%, about 30%, about 40%, about 50%, about 60%, about 70%, about 75%, about 80%, or about 90%. In some embodiments, the first or second compound reduces cancer cell viability by at least at most about 20%, about 25%, about 30%, about 40%, about 50%, about 60%, about 70%, about 75%, about 80%, about 90%, or about 100%.

In some embodiments, the first and second compounds synergistically reduce cancer cell viability. The synergistic effect may be accomplished when the first compound and the second compound are administered at the same time or at different times.

In some embodiments, the subject is an animal. Non-limiting examples of animals include mammals, such as a primates, cows, horses, goats, hogs, dogs, cats, or mice. In some embodiments, the subject is a mammal. In some embodiments, the subject is a primate. In some embodiments, the subject is a human.

### Cancer Symptoms

Cancers may include deadly diseases that often prove to be fatal, and the symptoms of cancer can often cause quality of life issues in subjects comprising a cancer. In some embodiments, the compositions, combinations and methods are used for treating a cancer symptom. In some embodiments, the compositions, combinations and methods are use used for lessening the severity of a cancer symptom. In some embodiments, the compositions, combinations and methods improve symptoms. In some embodiments, the disclosed treatment improves a cancer symptom in the subject, relative to a baseline cancer symptom. A baseline symptom may be measured at a time before the subject receives a cancer treatment, before the subject is diagnosed with a cancer, or after the subject receives a cancer treatment other than the compositions described herein. In some embodiments, the disclosed treatment improves a cancer symptom in the subject relative to a cancer symptom experienced while undergoing treatment with a different cancer therapy. Exemplary cancer symptoms include unexplained pain, fever, night sweats, fatigue, unexplained weight loss, weakness, anorexia, or unexplained bleeding. In some embodiments, the cancer symptom comprises a lymphoma symptom. Symptoms may also include those experienced from compression on organs by enlarged lymph nodes, lymphadenopathy, splenomegaly, cough, shortness of breath, chest discomfort, decreased appetite, nausea or vomiting, abdominal pain or swelling, feeling of fullness, rash or pruritis, alcohol intolerance, swelling in the face and arms, primary cutaneous lesions, neurologic symptoms, pain or superior vena cava syndrome. Additionally, cancer symptoms may include B symptoms. B symptoms refer to systemic symptoms of fever, night sweats, and weight loss, and are often associated with both Hodgkin and non-Hodgkin lymphoma. In some embodiments, the cancer symptom includes an enlarging mass at a single nodal region or extranodal sites. In some cases, the compositions, combinations and methods are used for the treatment of B symptoms.

Disclosed treatments may be useful for the treatment of or prevention of complications. In some embodiments, the disclosed treatment results in a lesser number or lesser severity of complications. Complications can occur due to the disease itself, or from the treatment of the disease. Often times, the treatment of cancers may involve complications that may include tumor lysis syndrome, febrile neutropenia, central nervous system recurrence, viral reactivation of hepatitis B, bleeding due to thrombocytopenia secondary to a therapy or disease, thrombosis, or progressive multifocal leukoencephalopathy. In some embodiments, the compositions, combinations and methods disclosed herein result in a change of prognosis. Treatments disclosed herein may be useful in lessening complications associated with previous treatment of a cancer due the present invention's ability to overcome resistance. Cancers exhibiting resistance to certain treatments may require higher doses of medications or may lead to the exposure of a treatment without any subsequent benefit. In either case, the compositions, combinations and methods described herein may be useful in lessening the severity or number of complications experienced by treatments to which the cancer is resistant. Additionally, the compositions, combinations and methods may be useful in lessening the complications associated with the cancer itself due to the ability of the treatments to treat the cancer, lessen the amount of cancer, or maintain the amount of cancer.

### Lab Results, Imaging, and Biopsy

In some embodiments, a lab result, image or biopsy is obtained from a subject, for example in making a measurement or determination that a method, composition, or combination described herein results in treatment of a cancer. In some embodiments, the subject is administered the composition when the lab result, image or biopsy is indicative of the presence of a cancer. In some embodiments, the subject is administered the composition when the lab result, image or biopsy is indicative of the presence of a lymphoma. The lab result, image or biopsy may be obtained to determine treatment effectiveness. In some embodiments, the lab result is obtained before the disclosed treatment is begun. In some embodiments, the lab result is obtained after the disclosed treatment has begun. In some embodiments, the compositions, combinations and methods result in an increase, decrease or maintain a value associated with a lab result, imaging, or biopsy. In some embodiments, a tissue sample is obtained from the subject. The tissue sample may be obtained before treatment, during treatment, or after treatment. In some embodiments, the disclosed treatment is administered after a lab result, image, or biopsy is obtained from a patient who received a different therapy. The lab result, image, or biopsy may demonstrate that the composition or method is beneficial in treating a cancer, that a cancer is not progressing, or that a cancer is regressing. Additionally, the lab result, image or biopsy may demonstrate that a previous treatment is not effective, or not as effective as desired. The biopsy sample may be a liquid biopsy, a bone marrow biopsy, lymph node or extranodal biopsy, cutaneous biopsy, stereotactic biopsy, skin biopsy, transbronchial or gastrointestinal tract biopsy, biopsy of other organs based on results of abnormal function tests, for example liver or kidney biopsy, or the biopsy may comprise excisional biopsy of lymph nodes, or needle core biopsy.

Non-limiting examples of imaging includes imaging with fluorodeoxyglucose positron emission tomography (FDG-PET)/computed tomography (CT), computed tomography, positron emission tomography (PET). Imaging of the chest, abdomen, neck, heard or pelvis may be conducted to help determine treatment effectiveness or disease responsiveness.

In some embodiments, the compositions, combinations and methods will be useful in reducing signs and symptoms of the cancer, such as anemia, thrombocytopenia, elevated lactate dehydrogenase, leukopenia, elevated erythrocyte sedimentation rate, hypoalbuminemia, and abnormalities associated with the liver, kidneys, or thyroids. In some embodiments, subject symptomology decreases in subjects receiving the disclosed treatment as compared to before the subjects received the disclosed treatment. In some embodiments, subject symptomology in subjects receiving the disclosed treatment decreases as compared to symptomology experienced with a different treatment regimen. In some embodiments, subject symptomology in subjects receiving the disclosed treatment decreases as compared to symptomology experienced when not receiving any treatment.

### Resistance

In some embodiments, the cancer is resistant to a treatment. In some embodiments, the composition or method may be useful in the treatment of a mixed cancer, such as in a patient comprising multiple primary cancers or a mix of primary and secondary cancers. In some embodiments, the cancer may be resistant to a previously received treatment. In some embodiments, the cancer is a relapsed or refractory cancer that is resistant to a treatment. In some embodiments, the composition or method is used to treat a resistant cancer. In some embodiments, the composition or method is used to treat a refractory cancer.

In some embodiments, the cancer is resistant to an EZH2 inhibitor. In some embodiments, the cancer is resistant to an immunomodulatory drug (IMiD). In some embodiments, the cancer is resistant to one or more EZH2 inhibitors and/or an immunomodulatory drug. In some embodiments, the cancer is refractory to one or more EZH2 inhibitors. In some embodiments, the cancer is refractory to one or more IMiDs. In some embodiments, the cancer is refractory to one or more EZH2 inhibitors and/or IMiDs. A cancer may be resistant to an EZH2 inhibitor such as EPZ-6438 (tazemetostat). The use of EZH2 inhibitors has demonstrated promise in the treatment of cancers; however, resistance to these drugs, such as with tazemetostat, has been identified as a problem. For example, in the treatment of DLBCL, studies have demonstrated the development of resistance to tazemetostat and GSK126. Therefore, there is a need to curtail resistance to EZH2 inhibitors. The treatments and methods provided herein may provide useful mechanisms for treating such a resistant cancer. Acquired resistance may arise in a recurring cancer or a refractory cancer and resistance may either be intrinsic or acquired. In cases involving resistant cancers, treatment options capable of overcoming the resistant are critical to treating the subject. Resistant cancers can be difficult to treat due to multiple factors and they can increase the complexity of treatment. Subjecting the subject comprising a cancer to multiple ineffective treatments can expose the subject to the risk of adverse drug effects without providing the benefit necessary for treating the cancer, and such ineffective treatments will cause the subject to potentially incur unnecessary costs. Recurring cancer can be difficult to treat, as the cancer may have developed resistance to the previous treatment and the subject may exhibit adverse effects related to the previous treatment. Previous treatments may not have been effective and may result in recurrent cancer. Throughout the disclosure, recurrent cancer and relapsed cancer are used synonymously.

Treatments useful in overcoming resistance may help lower costs associated with treating a cancer. The use of treatments to which a cancer is resistant can result in unnecessary costs and unnecessary exposure to adverse drug reactions. Therefore, treatments that overcome resistance may be useful in lowering unnecessary drug exposure. Additionally, providing a treatment option for a resistant cancer can help lower the number of different treatments to which the subject is exposed and/or reduce the total exposure of any one treatment to the subject. In either case, reducing exposure may be beneficial in preventing adverse drug reactions caused by such treatment. In some embodiments, the compositions, combinations and methods reduce the number of side effects and/or the severity of side effects experienced by treatment with previous treatments.

### Additional Cancer Treatments.

Cancer treatments may comprise the use of multiple different forms of treatment. In some cases, cancer treatments comprise the use of treatment regimen comprising multiple different cancer treatments. For example, subjects comprising a cancer are often treated with medications and radiation. The compositions, combinations and methods disclosed herein may be useful in the treatment of a cancer when used alone or in combination with another anti-cancer therapy. Although the compositions, combinations and methods described herein are useful in the treatment of a cancer, such as a resistant cancer, additional cancer treatments may be beneficial when used in combination with the disclosed compositions, combinations and methods. In some embodiments, the compositions, combinations and methods comprise one additional cancer treatment. In some embodiments, the compositions, combinations and methods comprise one or more additional cancer treatments. In some embodiments, the additional cancer treatment comprises chemotherapy, immunotherapy, radiation therapy, stem cell transplantation, or a targeted therapy. Exemplary additional cancer treatments include dexamethasone, cisplatin, cytarabine, rituximab, axicabtagene ciloleucel, tisagenlecleucel, bendamustine, brentuximab vedotin, ibrutinib, polatuzumab vedotin, Acalabrutinib, Copanlisib Hydrochloride, Nelarabine, Axicabtagene Ciloleucel, Belinostat, Bendamustine Hydrochloride, Carmustine, Bleomycin Sulfate, Bortezomib, Brexucabtagene Autoleucel, Lisocabtagene Maraleucel, Zanubrutinib, Acalabrutinib, Carmustine, Chlorambucil, Copanlisib Hydrochloride, Duvelisib, Crizotinib, Cyclophosphamide, Denileukin Diftitox, Doxorubicin Hydrochloride, Duvelisib, Pralatrexate, Obinutuzumab, Ibritumomab, Tiuxetan, Ibrutinib, Idelalisib, Ibrutinib, Recombinant Interferon Alfa-2b, Romidepsin, Pembrolizumab, Tisagenlecleucel, Chlorambucil, Lisocabtagene Maraleucel, Methotrexate Sodium, Mogamulizumab-kpkc, Tafasitamab-cxix, Plerixafor, Nelarabine, Obinutuzumab, Denileukin Diftitox, Pembrolizumab, Plerixafor, Polatuzumab Vedotin-piiq, Polatuzumab Vedotin-piiq, Mogamulizumab-kpkc, Pralatrexate, Prednisone, Recombinant Interferon Alfa-2b, Rituximab and Hyaluronidase Human, Rituximab, Romidepsin, Selinexor, Tafasitamab-cxix, Tazemetostat Hydrobromide, Brexucabtagene, Autoleucel, Tisagenlecleucel, Methotrexate Sodium, Umbralisib Tosylate, Bortezomib, Venetoclax, Vinblastine Sulfate, Vincristine Sulfate, Vorinostat, Crizotinib, Selinexor, Axicabtagene Ciloleucel, Zanubrutinib, Ibritumomab Tiuxetan and Idelalisib, or any combination thereof. In some embodiments, the additional cancer treatment comprises

In some embodiments, the first or second compound may also be included with any of the aforementioned treatments. In some embodiments, the composition comprises a first compound, a second compound and a third compound. In some embodiments, the composition comprises the first compound, the second compound, and one or more additional cancer treatments. In some embodiments, the composition comprises a first compound, a second compound, a third compound, and a fourth compound. The one or more additional cancer treatments, the third compound and the fourth compound can include any of the aforementioned treatments.

In some embodiments, the composition is a first line treatment for a cancer. In some embodiments, the composition is a second line treatment for a cancer. In some embodiments, the composition is a third line treatment for a cancer.

### Compositions and Kits

The compound may be formulated into a composition. Some embodiments relate to a composition comprising the compound. The composition may include any compound described herein. For example, the composition may include the compositions disclosed herein. The composition may include an EZH2 inhibitor and an IMiD. Administering a compound may comprise administering a composition comprising the compound. The composition may be a pharmaceutical composition.

In certain embodiments, the compound as described herein is administered as two pure chemicals. In other embodiments, the compound described herein is combined with a pharmaceutically acceptable carrier selected on the basis of a chosen route of administration.

In some embodiments, the composition is a pharmaceutical composition. In some embodiments, the composition is sterile. In some embodiments, the composition further comprises a pharmaceutically acceptable carrier.

In some embodiments, the pharmaceutically acceptable carrier comprises water. In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

In certain embodiments the compounds of the current disclosure are included in a pharmaceutical composition comprising one or more pharmaceutically acceptable excipients, carriers, and diluents. In certain embodiments, the compounds of the current disclosure are administered suspended in a sterile solution.

In certain embodiments, the compounds of the current disclosure are shipped/stored lyophilized and reconstituted before administration. In certain embodiments, lyophilized antibody compositions comprise a bulking agent such as, mannitol, sorbitol, sucrose, trehalose, dextran 40, or combinations thereof. The lyophilized composition can be contained in a vial comprised of glass or other suitable non-reactive material. The compounds when formulated into a composition, whether reconstituted or not, can be buffered at a certain pH, generally less than 7.0. In certain embodiments, the pH can be between 4.5 and 6.5, 4.5 and 6.0, 4.5 and 5.5, 4.5 and 5.0, or 5.0 and 6.0.

Also described herein are kits comprising one or more of the compounds described herein in a suitable container and one or more additional components selected from: instructions for use; a diluent, an excipient, a carrier, and a device for administration.

In certain embodiments, described herein is a method of preparing a cancer treatment comprising admixing one or more pharmaceutically acceptable excipients, carriers, or diluents and compounds of the current disclosure. In certain embodiments, described herein is a method of preparing a cancer treatment for storage or shipping comprising lyophilizing one or more components of the current disclosure.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes one sample or a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” “individual,” or “patient” are often used interchangeably herein. A “subject” can be a biological entity containing expressed genetic materials. The biological entity can be a plant, animal, or microorganism, including, for example, bacteria, viruses, fungi, and protozoa. The subject can be tissues, cells and their progeny of a biological entity obtained in vivo or cultured in vitro. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 15% of that number. The term “about” a range refers to that range minus 15% of its lowest value and plus 15% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkoxy” refers to a radical bonded through an oxygen atom of the formula —O-alkyl, where alkyl is an alkyl chain as defined above.

“Haloalkyl” refers to an alkyl group that is substituted by one or more halogens. Exemplary haloalkyl groups include trifluoromethyl, difluoromethyl, trichloromethyl, 2,2,2 trifluoroethyl, 1,2 difluoroethyl, 3 bromo 2 fluoropropyl, and 1,2 dibromoethyl.

“Heteroalkyl”, “heteroalkenyl” and “heteroalkynyl” refer to substituted or unsubstituted alkyl, alkenyl and alkynyl groups which respectively have one or more skeletal chain atoms selected from an atom other than carbon. Exemplary skeletal chain atoms selected from an atom other than carbon include, e.g., O, N, P, Si, S, or combinations thereof, wherein the nitrogen, phosphorus, and sulfur atoms may optionally be oxidized and the nitrogen heteroatom may optionally be quaternized. If given, a numerical range refers to the chain length in total. For example, a 3- to 8-membered heteroalkyl has a chain length of 3 to 8 atoms. Connection to the rest of the molecule may be through either a heteroatom or a carbon in the heteroalkyl, heteroalkenyl or heteroalkynyl chain. Unless stated otherwise specifically in the specification, a heteroalkyl, heteroalkenyl, or heteroalkynyl group is optionally substituted by one or more substituents such as those substituents described herein.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like. Unless stated otherwise specifically in the specification, an alkynyl group is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜORₐ (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₈ alkylene). In other embodiments, an alkylene comprises three to five carbon atoms (e.g., C₃-C₅ alkylene). Unless stated otherwise specifically in the specification, an alkylene chain is optionally substituted by one or more of the following substituents: halo, cyano, nitro, oxo, thioxo, imino, oximo, trimethylsilanyl, R^{a}, —OR^{a}, —SR^{a}, —OC(O)—R^{a}, —N(R^{a})₂, —C(O)R^{a}, —C(O)OR^{a}, —C(O)N(R^{a})₂, —N(R^{a})C(O)OR^{a}, —OC(O)—N(R^{a})₂, —N(R^{a})C(O)R^{a}, —N(R^{a})S(O)ₜR^{a} (where t is 1 or 2), —S(O)ₜOR^{a} (where t is 1 or 2), —S(O)ₜR^{a} (where t is 1 or 2) and —S(O)ₜN(R^{a})₂ (where t is 1 or 2) where each R^{a} is independently hydrogen, alkyl (optionally substituted with halogen, hydroxy, methoxy, or trifluoromethyl), fluoroalkyl, carbocyclyl (optionally substituted with halogen, hydroxy, methoxy, or trifluoromethyl), carbocyclylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl).

“Aryl” refers to a radical derived from an aromatic monocyclic or multicyclic hydrocarbon ring system by removing a hydrogen atom from a ring carbon atom. The aromatic monocyclic or multicyclic hydrocarbon ring system contains only hydrogen and carbon from five to eighteen carbon atoms, where at least one of the rings in the ring system is fully unsaturated, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. Unless stated otherwise specifically in the specification, the term “aryl” or the prefix “ar-” (such as in “aralkyl”) is meant to include aryl radicals optionally substituted by one or more substituents independently selected from alkyl, alkenyl, alkynyl, halo, fluoroalkyl, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“Aralkyl” refers to a radical of the formula —R-aryl where R is an alkylene chain as defined above, for example, methylene, ethylene, and the like. The alkylene chain part of the aralkyl radical is optionally substituted as described above for an alkylene chain. The aryl part of the aralkyl radical is optionally substituted as described above for an aryl group.

“Carbocyclyl” or “cycloalkyl” refers to a stable non-aromatic monocyclic or polycyclic hydrocarbon radical consisting solely of carbon and hydrogen atoms, which includes fused or bridged ring systems, having from three to fifteen carbon atoms. In certain embodiments, a carbocyclyl comprises three to ten carbon atoms. In other embodiments, a carbocyclyl comprises five to seven carbon atoms. The carbocyclyl is attached to the rest of the molecule by a single bond. Carbocyclyl is saturated (i.e., containing single C—C bonds only) or unsaturated (i.e., containing one or more double bonds or triple bonds). A fully saturated carbocyclyl radical is also referred to as “cycloalkyl.” Examples of monocyclic cycloalkyls include, e.g., cyclopropyl, cyclobutyl, cyclopentyl, cyclohexyl, cycloheptyl, and cyclooctyl. An unsaturated carbocyclyl is also referred to as “cycloalkenyl.” Examples of monocyclic cycloalkenyls include, e.g., cyclopentenyl, cyclohexenyl, cycloheptenyl, and cyclooctenyl. Polycyclic carbocyclyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless otherwise stated specifically in the specification, the term “carbocyclyl” is meant to include carbocyclyl radicals that are optionally substituted by one or more substituents independently selected from alkyl, alkenyl, alkynyl, halo, fluoroalkyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R is a straight or branched alkylene or alkenylene chain, and where each of the above substituents is unsubstituted unless otherwise indicated.

“Carbocyclylalkyl” refers to a radical of the formula —R^{c}-carbocyclyl where R^{c} is an alkylene chain as defined above. The alkylene chain and the carbocyclyl radical are optionally substituted as defined above.

“Halo” or “halogen” refers to bromo, chloro, fluoro or iodo substituents.

“Fluoroalkyl” refers to an alkyl radical, as defined above, that is substituted by one or more fluoro radicals, as defined above, for example, trifluoromethyl, difluoromethyl, fluoromethyl, 2,2,2-trifluoroethyl, 1-fluoromethyl-2-fluoroethyl, and the like. In some embodiments, the alkyl part of the fluoroalkyl radical is optionally substituted as defined above for an alkyl group.

“Heterocyclyl” or “heterocycloalkyl” refers to a stable 3- to 18-membered non-aromatic ring radical that comprises two to twelve carbon atoms and from one to six heteroatoms selected from nitrogen, oxygen and sulfur. Unless stated otherwise specifically in the specification, the heterocyclyl radical is a monocyclic, bicyclic, tricyclic or tetracyclic ring system, which optionally includes fused or bridged ring systems. The heteroatoms in the heterocyclyl radical are optionally oxidized. One or more nitrogen atoms, if present, are optionally quaternized. The heterocyclyl radical is partially or fully saturated. The heterocyclyl is attached to the rest of the molecule through any atom of the ring(s). Examples of such heterocyclyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl. Unless stated otherwise specifically in the specification, the term “heterocyclyl” is meant to include heterocyclyl radicals as defined above that are optionally substituted by one or more substituents selected from alkyl, alkenyl, alkynyl, halo, fluoroalkyl, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R^{c} is a straight or branched alkylene or alkenylene chain, and where each of the above substituents is unsubstituted unless otherwise indicated.

“N-heterocyclyl” or “N-attached heterocyclyl” refers to a heterocyclyl radical as defined above containing at least one nitrogen and where the point of attachment of the heterocyclyl radical to the rest of the molecule is through a nitrogen atom in the heterocyclyl radical. An N-heterocyclyl radical is optionally substituted as described above for heterocyclyl radicals. Examples of such N-heterocyclyl radicals include, but are not limited to, 1-morpholinyl, 1-piperidinyl, 1-piperazinyl, 1-pyrrolidinyl, pyrazolidinyl, imidazolinyl, and imidazolidinyl.

“C-heterocyclyl” or “C-attached heterocyclyl” refers to a heterocyclyl radical as defined above containing at least one heteroatom and where the point of attachment of the heterocyclyl radical to the rest of the molecule is through a carbon atom in the heterocyclyl radical. A C-heterocyclyl radical is optionally substituted as described above for heterocyclyl radicals. Examples of such C-heterocyclyl radicals include, but are not limited to, 2-morpholinyl, 2- or 3- or 4-piperidinyl, 2-piperazinyl, 2- or 3-pyrrolidinyl, and the like.

“Heteroaryl” refers to a radical derived from a 3- to 18-membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is fully unsaturated, i.e., it contains a cyclic, delocalized (4n+2) π-electron system in accordance with the Hickel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzooxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl, benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a,7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pridinyl, and thiophenyl (i.e. thienyl). Unless stated otherwise specifically in the specification, the term “heteroaryl” is meant to include heteroaryl radicals as defined above which are optionally substituted by one or more substituents selected from alkyl, alkenyl, alkynyl, halo, fluoroalkyl, haloalkenyl, haloalkynyl, oxo, thioxo, cyano, nitro, optionally substituted aryl, optionally substituted aralkyl, optionally substituted aralkenyl, optionally substituted aralkynyl, optionally substituted carbocyclyl, optionally substituted carbocyclylalkyl, optionally substituted heterocyclyl, optionally substituted heterocyclylalkyl, optionally substituted heteroaryl, optionally substituted heteroarylalkyl, R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2), where each R^{a} is independently hydrogen, alkyl (optionally substituted with halogen, hydroxy, methoxy, or trifluoromethyl), fluoroalkyl, cycloalkyl (optionally substituted with halogen, hydroxy, methoxy, or trifluoromethyl), cycloalkylalkyl (optionally substituted with halogen, hydroxy, methoxy, or trifluoromethyl), aryl (optionally substituted with halogen, hydroxy, methoxy, or trifluoromethyl), aralkyl (optionally substituted with halogen, hydroxy, methoxy, or trifluoromethyl), heterocyclyl (optionally substituted with halogen, hydroxy, methoxy, or trifluoromethyl), heterocyclylalkyl (optionally substituted with halogen, hydroxy, methoxy, or trifluoromethyl), heteroaryl (optionally substituted with halogen, hydroxy, methoxy, or trifluoromethyl), or heteroarylalkyl (optionally substituted with halogen, hydroxy, methoxy, or trifluoromethyl), each R^{b} is independently a direct bond or a straight or branched alkylene or alkenylene chain, and R is a straight or branched alkylene or alkenylene chain, and where each of the above substituents is unsubstituted unless otherwise indicated.

“N-heteroaryl” refers to a heteroaryl radical as defined above containing at least one nitrogen and where the point of attachment of the heteroaryl radical to the rest of the molecule is through a nitrogen atom in the heteroaryl radical. An N-heteroaryl radical is optionally substituted as described above for heteroaryl radicals.

“C-heteroaryl” refers to a heteroaryl radical as defined above and where the point of attachment of the heteroaryl radical to the rest of the molecule is through a carbon atom in the heteroaryl radical. A C-heteroaryl radical is optionally substituted as described above for heteroaryl radicals.

The compounds disclosed herein, in some embodiments, contain one or more asymmetric centers and thus give rise to enantiomers, diastereomers, and other stereoisomeric forms that are defined, in terms of absolute stereochemistry, as (R)- or (S)-. Unless stated otherwise, it is intended that all stereoisomeric forms of the compounds disclosed herein are contemplated by this disclosure. When the compounds described herein contain alkene double bonds, and unless specified otherwise, it is intended that this disclosure includes both E and Z geometric isomers (e.g., cis or trans.) Likewise, all possible isomers, as well as their racemic and optically pure forms, and all tautomeric forms are also intended to be included. The term “geometric isomer” refers to E or Z geometric isomers (e.g., cis or trans) of an alkene double bond. The term “positional isomer” refers to structural isomers around a central ring, such as ortho-, meta-, and para-isomers around a benzene ring.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

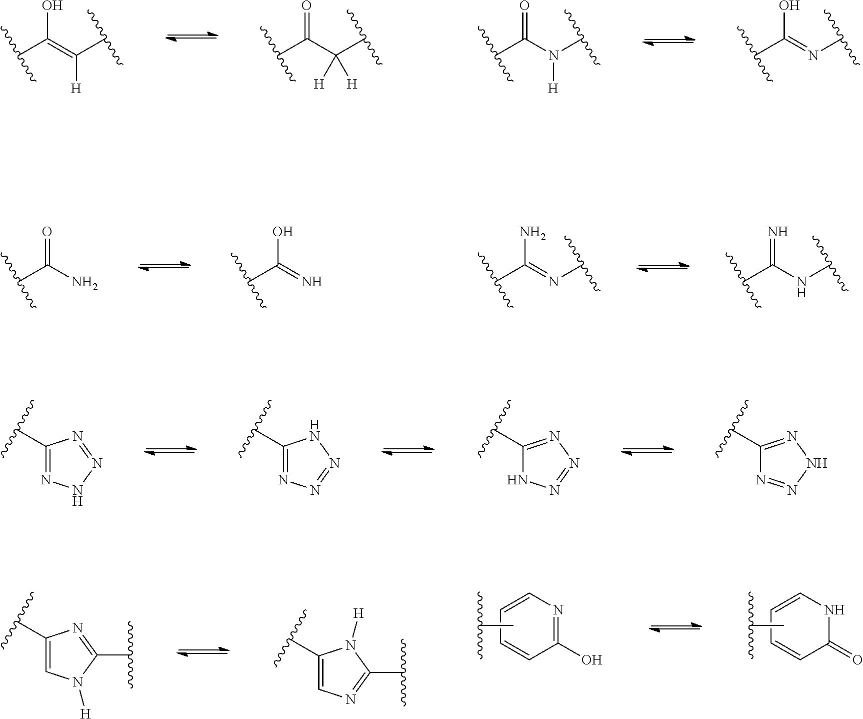

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, structures depicted herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

“Pharmaceutically acceptable salt” includes both acid and base addition salts. A pharmaceutically acceptable salt of any one of the compounds described herein is intended to encompass any and all pharmaceutically suitable salt forms. Preferred pharmaceutically acceptable salts of the compounds described herein are pharmaceutically acceptable acid addition salts and pharmaceutically acceptable base addition salts.

“Pharmaceutically acceptable acid addition salt” refers to those salts which retain the biological effectiveness and properties of the free bases, which are not biologically or otherwise undesirable, and which are formed with inorganic acids such as hydrochloric acid, hydrobromic acid, sulfuric acid, nitric acid, phosphoric acid, hydroiodic acid, hydrofluoric acid, phosphorous acid, and the like. Also included are salts that are formed with organic acids such as aliphatic mono- and dicarboxylic acids, phenyl-substituted alkanoic acids, hydroxy alkanoic acids, alkanedioic acids, aromatic acids, aliphatic and. aromatic sulfonic acids, etc. and include, for example, acetic acid, trifluoroacetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Exemplary salts thus include sulfates, pyrosulfates, bisulfates, sulfites, bisulfites, nitrates, phosphates, monohydrogenphosphates, dihydrogenphosphates, metaphosphates, pyrophosphates, chlorides, bromides, iodides, acetates, trifluoroacetates, propionates, caprylates, isobutyrates, oxalates, malonates, succinate suberates, sebacates, fumarates, maleates, mandelates, benzoates, chlorobenzoates, methylbenzoates, dinitrobenzoates, phthalates, benzenesulfonates, toluenesulfonates, phenylacetates, citrates, lactates, malates, tartrates, methanesulfonates, and the like. Also contemplated are salts of amino acids, such as arginates, gluconates, and galacturonates (see, for example, Berge S. M. et al., “Pharmaceutical Salts,” Journal of Pharmaceutical Science, 66:1-19 (1997)). Acid addition salts of basic compounds are, in some embodiments, prepared by contacting the free base forms with a sufficient amount of the desired acid to produce the salt according to methods and techniques with which a skilled artisan is familiar.

“Pharmaceutically acceptable base addition salt” refers to those salts that retain the biological effectiveness and properties of the free acids, which are not biologically or otherwise undesirable. These salts are prepared from addition of an inorganic base or an organic base to the free acid. Pharmaceutically acceptable base addition salts are, in some embodiments, formed with metals or amines, such as alkali and alkaline earth metals or organic amines. Salts derived from inorganic bases include, but are not limited to, sodium, potassium, lithium, ammonium, calcium, magnesium, iron, zinc, copper, manganese, aluminum salts and the like. Salts derived from organic bases include, but are not limited to, salts of primary, secondary, and tertiary amines, substituted amines including naturally occurring substituted amines, cyclic amines and basic ion exchange resins, for example, isopropylamine, trimethylamine, diethylamine, triethylamine, tripropylamine, ethanolamine, diethanolamine, 2-dimethylaminoethanol, 2-diethylaminoethanol, dicyclohexylamine, lysine, arginine, histidine, caffeine, procaine, N,N-dibenzylethylenediamine, chloroprocaine, hydrabamine, choline, betaine, ethylenediamine, ethylenedianiline, N-methylglucamine, glucosamine, methylglucamine, theobromine, purines, piperazine, piperidine, N-ethylpiperidine, polyamine resins and the like. See Berge et al., supra.

### Embodiments

In some preferred embodiments, the invention provides embodiments E1 to E49.
E1. A method of treatment, comprising:
administering to a subject in need of a cancer treatment, a first compound comprising a compound of Formula I:

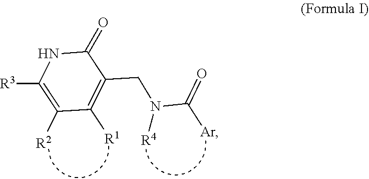

or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
administering to the subject a second compound comprising an immunomodulatory drug.
E2. A composition for use in treating a subject in need of a cancer treatment, comprising:
   a first compound comprising a compound of Formula I:

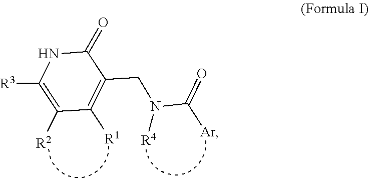

or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
   R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
   two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
   a second compound comprising an immunomodulatory drug.
E3. A combination therapy comprising:
   a first compound comprising a compound of Formula I:

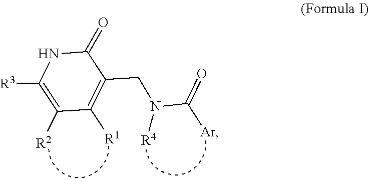

or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R, OR, SR, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and a second compound comprising an immunomodulatory drug.
E4. The method, composition, or combination of embodiment E1, E2 or E3, wherein the first compound is an Enhancer of Zeste Homolog 2 (EZH2) inhibitor.
E5. The method, composition, or combination of embodiment E4, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.
E6. The method, composition, or combination of any one of embodiments E1 to E5, wherein the immunomodulatory drug comprises an immunomodulatory imide drug (IMiD).
E7. The method, composition, or combination of embodiment E6, wherein the IMiD comprises a cereblon modulator.
E8. The method, composition, or combination of embodiment E7, wherein the cereblon modulator comprises a compound of Formula II:

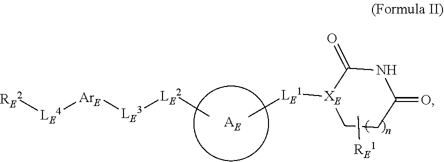

or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring; L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring; Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and n is selected from 0, 1, and 2.
E9. The method, composition, or combination of embodiment E7 or E8, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.
E10. The method, composition, or combination of embodiment E7 or E8, wherein the cereblon modulator excludes thalidomide, lenalidomide, and/or pomalidomidedomide.
E11. The method, composition, or combination of embodiment E7 or E8, wherein the cereblon modulator is an IKZF1 and/or IZKF3 degrader or modulator.
E12. The method, composition, or combination of embodiment E11, wherein the cereblon modulator is an IKZF1/3 degrader or modulator.
E13. A method of treatment, comprising:
   administering to a subject in need of a cancer treatment, a first compound comprising an EZH2 inhibitor; and
   administering to the subject a second compound comprising a compound of Formula II:

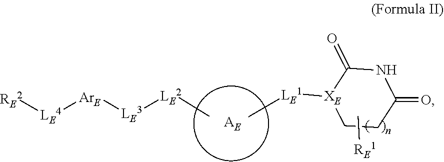

or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.
E14. A composition for use in treating a subject in need of a cancer treatment, comprising:
   a first compound comprising an EZH2 inhibitor; and
   a second compound comprising a compound of Formula II:

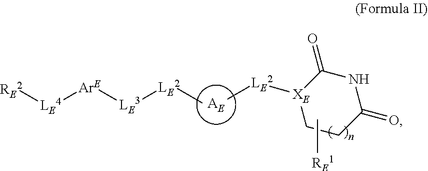

or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.
E15. A combination, comprising:
   a first compound comprising an EZH2 inhibitor; and
   a second compound comprising a compound of Formula II:

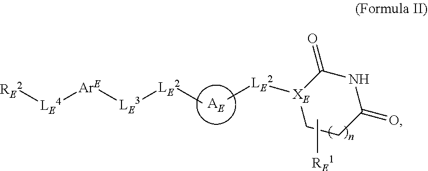

or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}b is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.
E16. The method, composition, or combination of any one of embodiments E13 to E15, wherein the EZH2 inhibitor comprises a compound of Formula I:

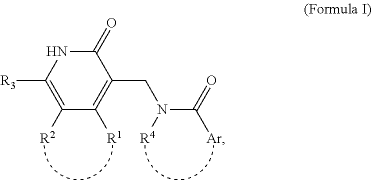

or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
   R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
   two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.
E17. The method, composition, or combination of any one of embodiments E13 to E16, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.
E18. The method, composition, or combination of any one of embodiments E13 to E17, wherein the compound of Formula II is a cereblon modulator.
E19. The method, composition, or combination of embodiment E18, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.
E20. The method, composition, or combination of embodiment E18, wherein the cereblon modulator excludes thalidomide, lenalidomide, and/or pomalidomide.
E21. The method, composition, or combination of embodiment E18, wherein the cereblon modulator is an IKZF1 and/or IZKF3 degrader or modulator.
E22. The method, composition, or combination of embodiment E18 or E21, wherein the cereblon modulator is an IKZF1/3 degrader or modulator.
E23. The method, composition, or combination of any one of embodiments E1 to E22, wherein the cancer is resistant to an EZH2 inhibitor or an IMiD.
   E24. A method of treatment, comprising:
   identifying a subject having a relapsed or refractory cancer resistant to one or more EZH2 inhibitors or IMiDs;
   administering to the subject a first compound comprising an EZH2 inhibitor; and
   administering to the subject a second compound comprising an IMiD.
E25. A composition for use in treating a subject identified as having a relapsed or refractory cancer resistant to one or more EZH2 inhibitors or IMiDs, comprising:
   a first compound comprising an EZH2 inhibitor; and
   a second compound comprising an IMiD.
E26. A combination for use in treating relapsed or refractory cancer resistant to one or more EZH2 inhibitors or IMiDs, comprising:
   a first compound comprising an EZH2 inhibitor; and
   a second compound comprising an IMiD.
E27. The method, composition, or combination of any one of embodiments E24 to E26, wherein the EZH2 inhibitor comprises a compound of Formula I:

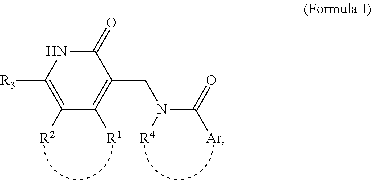

or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.
E28. The method, composition, or combination of any one of embodiments E24 to E27, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.
E29. The method, composition, or combination of any one of embodiments E24 to E28, wherein the IMiD comprises a cereblon modulator.
E30. The method, composition, or combination of embodiment E29, wherein the cereblon modulator comprises a compound of Formula II:

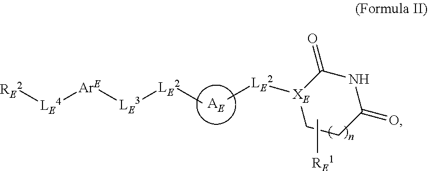

or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-.
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.
E31. The method, composition, or combination of embodiment E29 or E30, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.
E32. The method, composition, or combination of embodiment E29 or E30, wherein the cereblon modulator excludes thalidomide, lenalidomide, and/or pomalidomide.
E33. The method, composition, or combination of embodiment E29 or E30, wherein the cereblon modulator is an IKZF1 and/or IZKF3 degrader or modulator.
E34. The method, composition, or combination of embodiment E11, wherein the cereblon modulator is an IKZF1/3 degrader or modulator.
E235. The method, composition, or combination of any one of the aforementioned embodiments, wherein the first or second compound reduces cancer cell viability.
E36. The method, composition, or combination of any one of the aforementioned embodiments, wherein the first and second compounds synergistically reduce cancer cell viability.
E37. The method, composition, or combination of embodiment E35 or E36, wherein the cancer cell viability is reduced by at least 10%, at least 20%, or at least 50%.
E38. The method, composition, or combination of any one of embodiments E35 to E37, wherein administration of the first and second compounds decreases the cancer cell viability in the subject.
E39. The method, composition, or combination of any one of the aforementioned embodiments, wherein administration of the first and second compounds improves a cancer symptom in the subject, relative to a baseline cancer symptom.
E40. The method, composition, or combination of embodiment E39, wherein the cancer symptom includes uncontrolled cell growth or division, unexplained pain, fever, night sweats, fatigue, unexplained weight loss, weakness, anorexia, or unexplained bleeding.
E41. The method, composition, or combination of any one of the aforementioned embodiments, wherein the cancer comprises a blood cancer.
E42. The method, composition, or combination of embodiment E41, wherein the blood cancer comprises a lymphoma, multiple myeloma, or leukemia.
E43. The method, composition, or combination of embodiment E41, wherein the blood cancer comprises lymphoma.
E44. The method, composition, or combination of embodiment E41 or E42, wherein the lymphoma comprises a non-Hodgkin's lymphoma.
E45. The method, composition, or combination of embodiment E44, wherein the non-Hodgkin's lymphoma is follicular lymphoma (FL) or diffuse large B-cell lymphoma (DLBCL).
E46. The method, composition, or combination of any one of the aforementioned embodiments, further comprising administering an additional cancer treatment to the subject.
E47. The method, composition, or combination of any one of the aforementioned embodiments, wherein the additional cancer treatment comprises chemotherapy, immunotherapy, radiation therapy, stem cell transplantation, targeted therapy.
E48. The method, composition, or combination of any one of the aforementioned embodiments, wherein the subject is a mammal.
E49. The method, composition, or combination of any one of the aforementioned embodiments, wherein the subject is human.

### Examples

The following examples are included for illustrative purposes only and are not necessarily intended to limit the scope of the invention.

### Example 1: Combinations of EZH2 Inhibitors and IMiDs

EZH2 and the targets of IMiDs, such as IKZF1 and IKZF3, may be implicated in both lymphocyte development and lymphogenesis. Both EZH2 inhibitors and IMiDs may have clinical activities in a variety of lymphoma subtypes, but the benefits of these therapeutics in patients when administered alone may be modest. On the other hand, the combination of EZH2 inhibitors and IMiDs may be more effective.

### Cell Culture

Experiments were performed in cultured human cells to assess the efficacy of combined EZH2 inhibitor and immunomodulatory drug (e.g., an IMiD) treatment using an EZH2-mutant DLBCL cell line, SU-DHL-10, and an EZH2-wild type line, OCI-LY7. Cells were purchased from Nanjing Cobioer Biotechnology. SU-DHL-10 and OCI-LY7 cells were cultured at 37° C. with 5% CO₂ in RPMI 1640 Medium supplemented with 10% fetal bovine serum. Cells were authenticated using the short tandem repeat (STR) assays. Mycoplasma test results were negative.

### Cell Viability Assays

The cultured cells were seeded at a density of 5000 cells per well in 96-well assay plates and treated with test compounds at 10 μM followed by a 11-point 3-fold serial dilution or an 8-point 10-fold serial dilution. Cell viability was determined after 5 days, using the CellTiter-Glo assay kit (Promega) according to the manufacturer's instructions. The dose-response curves were determined and IC₅₀ values were calculated using the GraphPad Prism software following a nonlinear regression (least squares fit) method. Each data point in the figure represents the mean values of at least two technical replicates ± standard deviation.

Using SU-DHL-10 and OCI-LY7 cells, none of the tested EZH2 inhibitors and IMiDs effectively decreased cell viability as single agents. Cells were treated with EZH2 inhibitors in the presence of IMiDs at concentrations sufficient to induce degradation of IKZF1 and IKZF3 but not enough to kill cells. CC-122 and CC-220 were tested at a concentration of 1 μM; CC-92480 and CFT7455 were tested at a concentration of 1 nM; and CC-99282 was tested at a concentration of 10 nM.

SU-DHL-10 and OCI-LY7 cells were highly sensitive to EPZ-6438 (tazemetostat), GSK126 and CPI-1205, when combined with any of the IMiDs CC-122, CC-220 or CC-92480. SU-DHL-10 and OCI-LY7 cells were highly sensitive to CPI-0209 and SHR2554 when combined with the IMiDs CC-99282 or CFT7455.

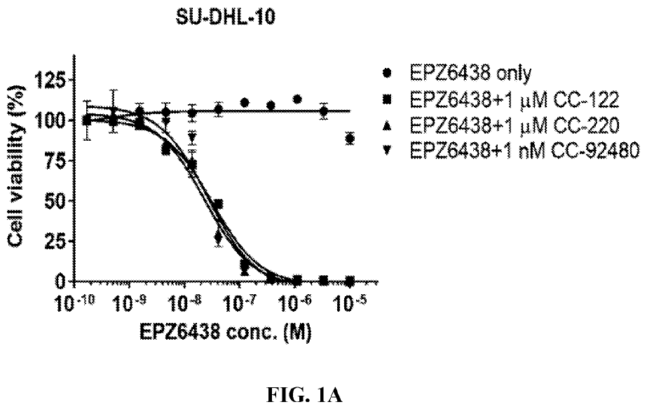

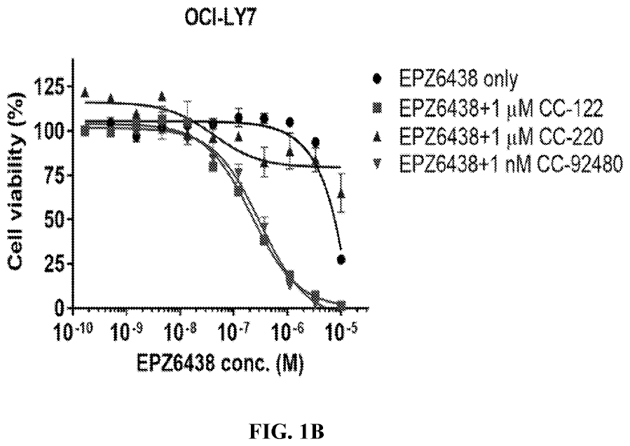

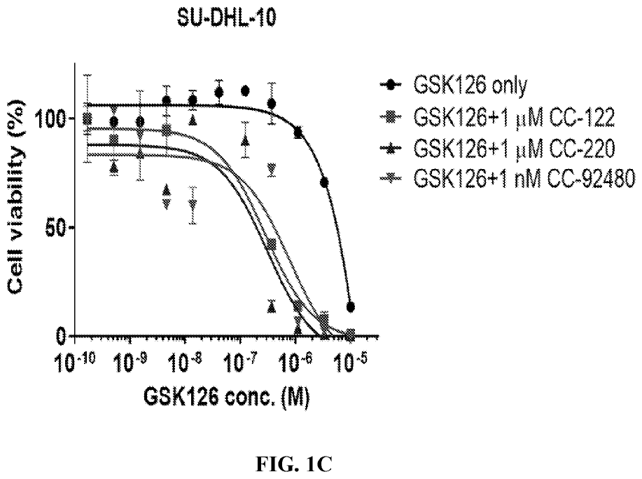

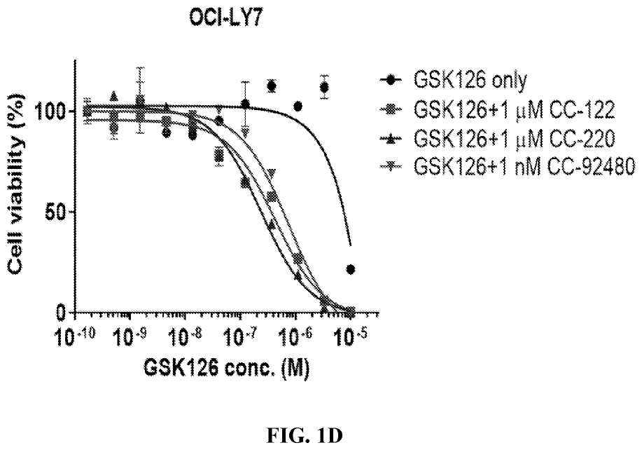

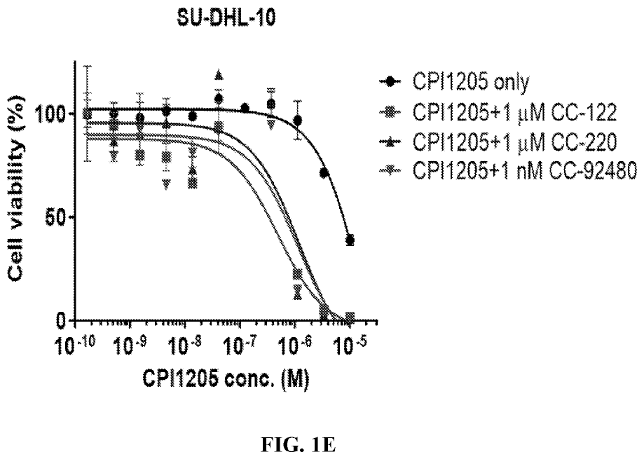

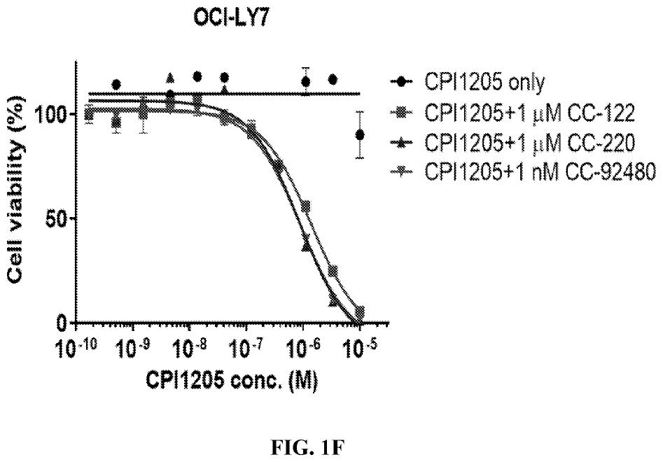

FIG. 1 demonstrates that the combination of EZH2 inhibitors with IMiDs improved lymphoma sensitivity relative to EZH2 inhibitors alone in SU-DHL-10 and OCI-LY7 cells. Cells were treated with EPZ-6438, GSK126 or CPI-1205 at 10 μM followed by an 11-point 3-fold serial dilution, in the absence or presence of 1 μM CC-122, 1 μM CC-220, or 1 nM CC-92480. Cells were incubated for 5 days prior to cell viability assays.

FIG. 1A shows that the combination of EPZ-6438 with an IMiD improved lymphoma sensitivity in SU-DHL-10 cells. The combination of EPZ-6438 with any of CC-122, CC-220, or CC-92480 lowered cell viability more than treatment with EPZ-6438 alone.

FIG. 1B demonstrates that different combinations of EPZ-6438 with an IMiD improved lymphoma sensitivity in OCI-LY7 cells. The combination of EPZ-6438 with any of CC-122 or CC-92480 lowered cell viability more than treatment with EPZ-6438 alone.

FIG. 1C demonstrates that different combinations of GSK126 with an IMiD improved lymphoma sensitivity in SU-DHL-10 cells. The combination of GSK126 with any of CC-122, CC-220, or CC-92480 lowered cell viability more than treatment with GSK126 alone.

FIG. 1D demonstrates that different combinations of GSK126 with an IMiD improved lymphoma sensitivity in OCI-LY7 cells. The combination of GSK126 with any of CC-122, CC-220, or CC-92480 lowered cell viability more than treatment with GSK126 alone.

FIG. 1E demonstrates that different combinations of CPI-1205 with an IMiD improved lymphoma sensitivity in SU-DHL-10 cells. The combination of CPI-1205 with any of CC-122, CC-220, or CC-92480 lowered cell viability more than treatment with CPI-1205 alone.

FIG. 1F demonstrates that different combinations of CPI-1205 with an IMiD improved lymphoma sensitivity in OCI-LY7 cells. The combination of CPI-1205 with any of CC-122, CC-220, or CC-92480 lowered cell viability more than treatment with CPI-1205 alone.

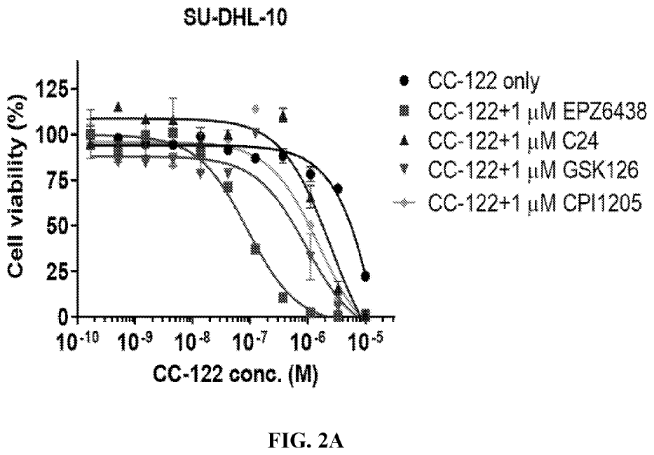

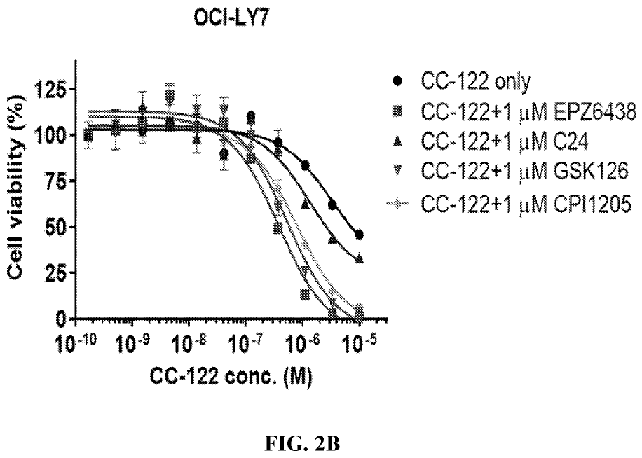

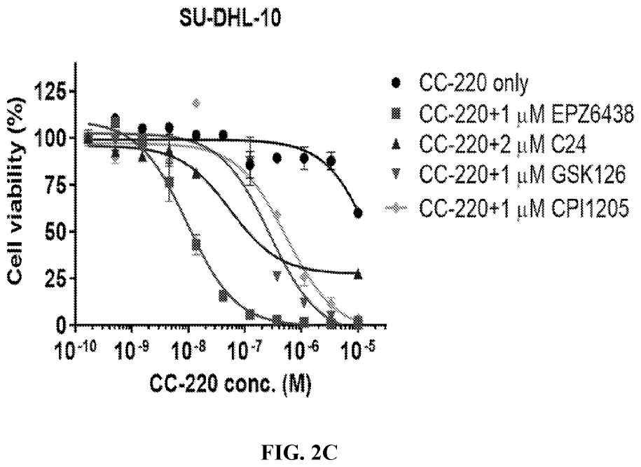

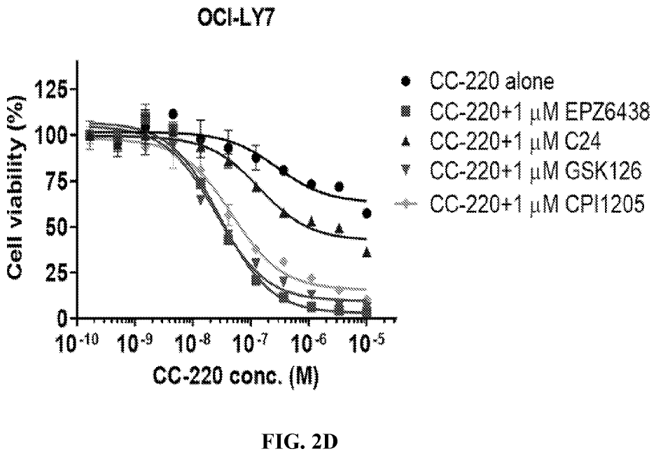

FIG. 2 demonstrated that EZH2 inhibitors improve lymphoma sensitivity to IMiDs. SU-DHL-10 and OCI-LY7 cells were treated with EZH2 inhibitors, EPZ-6438, GSK126 or CPI-1205, at M followed by a 11-point 3-fold serial dilution, in the absence or presence of 1 μM CC-122, 1 M CC-220, or 1 nM CC-92480. Cells were incubated for 5 days prior to cell viability assays. The combinations of the IMiDs and EZH2 inhibitors improved lymphoma sensitivity relative to the IMiD alone in SU-DHL-10 and OCI-LY7 cells. SU-DHL-10 cells were refractory to CC-122 and CC-220, while OCI-LY7 cells were modestly responsive to CC-122 and CC-220 (FIGS. 2A-2D). Both SU-DHL-10 and OCI-LY7 cells were highly sensitive to CC-122 and CC-220 in the presence of EPZ-6438, C24, GSK126 or CPI-1205 (FIGS. 2A-2D). The potent EZH2 inhibitor, EPZ-6438, resulted in the most dramatic improvement of cellular sensitivity in combination with CC-122 and CC-220. More specifically, in the presence of 1 μM EPZ-6438, the IC₅₀ value of CC-122 was 89 nM in SU-DHL-10 cells and 380 nM in OCI-LY7 cells. The more potent IKZF1/3 degrader CC-220 had IC₅₀ values of 9.5 nM and 30 nM in SU-DHL-10 and OCI-LY7 cell lines, respectively.

FIG. 2A demonstrated that different combinations of CC-122 with an IMiD improved lymphoma sensitivity in SU-DHL-10 cells. The combination of CC-122 with any of CC-122, CC-220, or CC-92480 lowered cell viability more than treatment with CC-122 alone.

FIG. 2B demonstrated that different combinations of CC-122 with an IMiD improved lymphoma sensitivity in OCI-LY7 cells. The combination of CC-122 with any of CC-122, CC-220, or CC-92480 lowered cell viability more than treatment with CC-122 alone.

FIG. 2C demonstrated that different combinations of CC-220 with an IMiD improved lymphoma sensitivity in SU-DHL-10 cells. The combination of CC-220 with any of CC-122, CC-220, or CC-92480 lowered cell viability more than treatment with CC-220 alone.

FIG. 2D demonstrated that different combinations of CC-220 with an IMiD improved lymphoma sensitivity in OCI-LY7 cells. The combination of CC-220 with any of CC-122, CC-220, or CC-92480 lowered cell viability more than treatment with CC-220 alone.

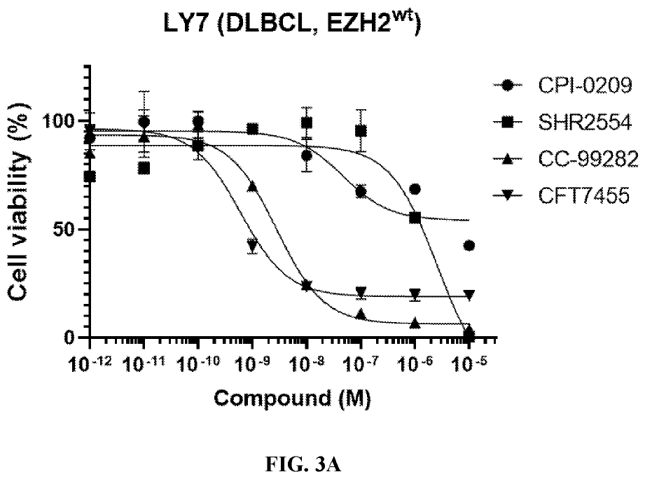

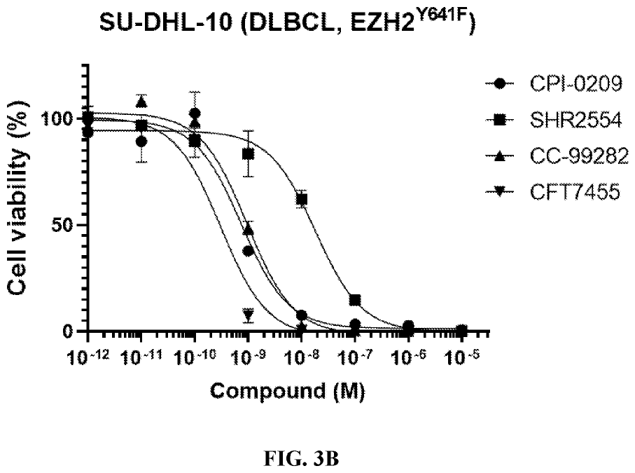

FIG. 3 shows the effect of single agent treatment with EZH2 inhibitors and IMiDs on cell viability in SU-DHL-10 and OCI-LY7 cells. Compounds were tested at a concentration 10 μM followed by an 8-point 10-fold serial dilution. OCI-LY7 cells were refractory to SHR2554, modestly responsive to CPI-0209, and moderately responsive to CC-99282 and CFT7455 (FIG. 3A). SU-DHL-10 cells were modestly responsive to SHR2554, and moderately responsive to CPI-0209, CC-99282 and CFT7455 (FIG. 3B).

FIG. 3A shows single-agent activities of two EZH2 inhibitors (CPI-0209 and SHR2554) and two IKZF1/3 degraders (CC-99282 and CFT7455) in OCI-LY7 cells after 5 days of treatment.

FIG. 3B shows single-agent activities of two EZH2 inhibitors (CPI-0209 and SHR2554) and two IKZF1/3 degraders (CC-99282 and CFT7455) in SU-DHL-10 cells after 5 days of treatment.

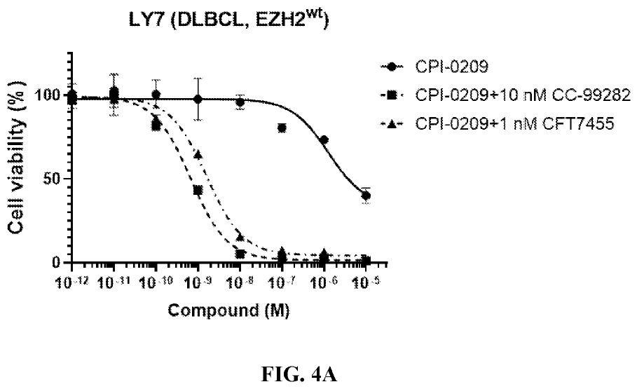

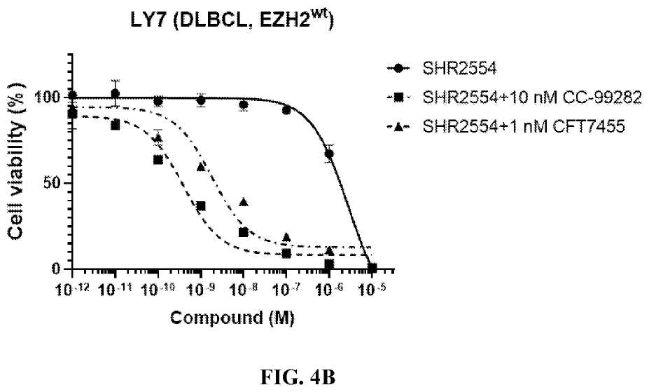

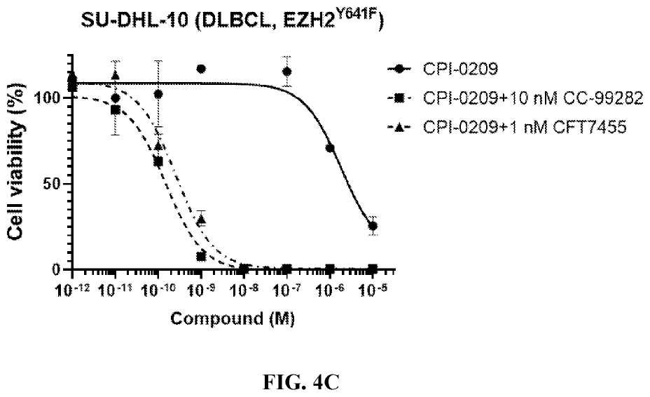

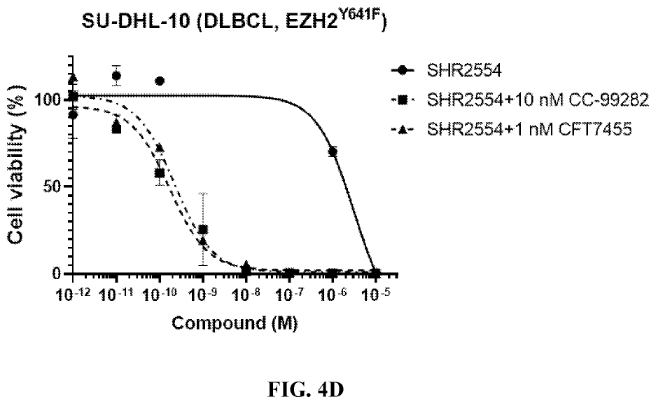

FIG. 4 further demonstrates that IMiDs improved lymphoma sensitivity to EZH2 inhibitors in SU-DHL-10 and OCI-LY7 cells. Cells were treated with EZH2 inhibitors, CPI-0209 and SHR2554, at 10 μM followed by an 8-point 10-fold serial dilution, in the absence or presence of 10 nM CC-99282 or 1 nM CFT7455. Cells were incubated for 5 days prior to cell viability assays.

FIG. 4A demonstrates that different combinations of CPI-0209 with an IMiD improved lymphoma sensitivity in OCI-LY7 cells. The combination of CPI-0209 with CC-99282 or CFT7455 lowered cell viability more than treatment with CPI-0209 alone.

FIG. 4B demonstrates that different combinations of SHR2554 with an IMiD improved lymphoma sensitivity in OCI-LY7 cells. The combination of SHR2554 with CC-99282 or CFT7455 lowered cell viability more than treatment with SHR2554 alone.

FIG. 4C demonstrates that different combinations of CPI-0209 with an IMiD improved lymphoma sensitivity in SU-DHL-10 cells. The combination of CPI-0209 with CC-99282 or CFT7455 lowered cell viability more than treatment with CPI-0209 alone.

FIG. 4D demonstrates that different combinations of SHR2554 with an IMiD improved lymphoma sensitivity in SU-DHL-10 cells. The combination of SHR2554 with CC-99282 or CFT7455 lowered cell viability more than treatment with SHR2554 alone.

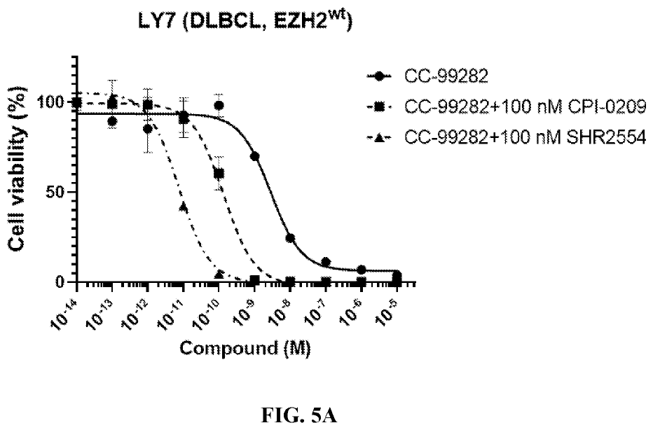

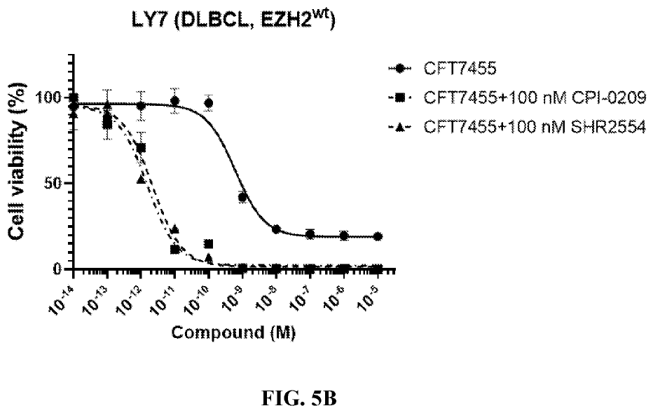

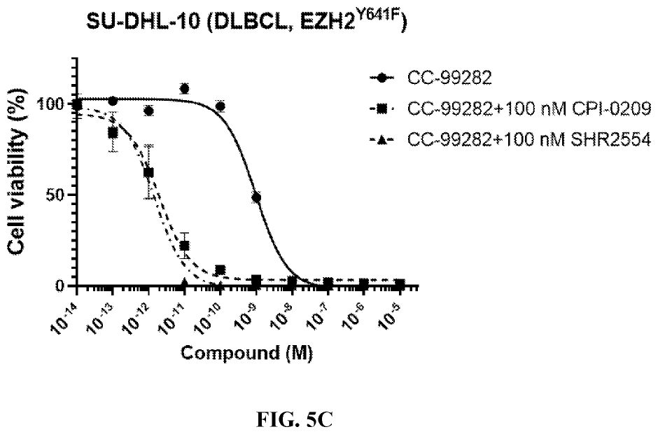

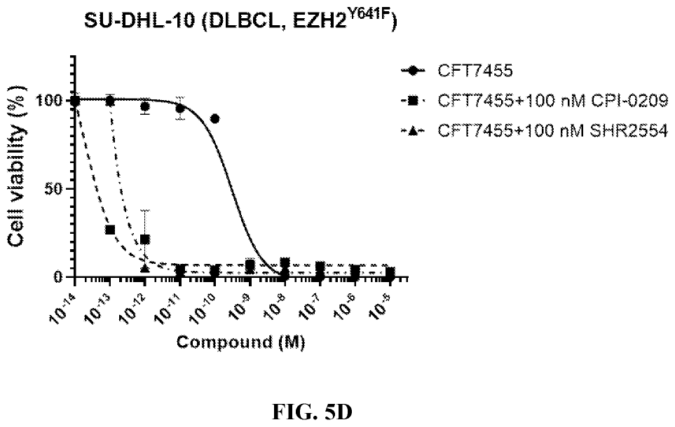

FIG. 5 further demonstrates that that IMiDs improve lymphoma sensitivity to EZH2 inhibitors in SU-DHL-10 and OCI-LY7 cells. Cells were treated with IMiDs CC-99282 or CFT7455, at 10 μM followed by an 8-point 10-fold serial dilution, in the absence or presence of 100 nM CPI-0209 or 100 nM SHR2554.

FIG. 5A demonstrates that different combinations of CC-99282 with an EZH2 inhibitor improved lymphoma sensitivity in OCI-LY7 cells. The combination of CC-99282 with CPI-0209 or SHR2554 lowered cell viability more than treatment with CC-99282 alone.

FIG. 5B demonstrates that different combinations of CFT7455 with an EZH2 inhibitor improved lymphoma sensitivity in OCI-LY7 cells. The combination of CFT7455 with CPI-0209 or SHR2554 lowered cell viability more than treatment with CFT7455 alone.

FIG. 5C shows that different combinations of CC-99282 with an EZH2 inhibitor improved lymphoma sensitivity in SU-DHL-10 cells. The combination of CC-99282 with CPI-0209 or SHR2554 lowered cell viability more than treatment with CC-99282 alone.

FIG. 5D shows that different combinations of CFT7455 with an EZH2 inhibitor improved lymphoma sensitivity in SU-DHL-10 cells. The combination of CFT7455 with CPI-0209 or SHR2554 lowered cell viability more than treatment with CFT7455 alone.

The Examples provided herein show that the combination of EZH2 inhibitors and IMiDs provided synergistic effects and was significantly more potent than either class of agents alone in SU-DHL-10 cells and OCI-LY7 cells. Such combinations may be useful for the treatment of lymphoma, irrespective of EZH2 mutational status. The data demonstrate that the combination of the EZH2 inhibitor and IMiD provided effects that resulted in lower cancer cell viability as compared to the use of the individual components alone. Combinations of an EZH2 inhibitor and an IMiD lowered the viability of cells comprising B cell lymphoma, such as diffuse large B-cell lymphoma, demonstrated by the lowered cell viability of SU-DHL-10 cells and OCI-LY7 cells, which comprise B cell lymphoma, such as diffuse large B-cell lymphoma.

Therefore, such combinations of EZH2 inhibitors and IMiDs may be useful in treating cancers, such as lymphoma or another blood cancer. The data demonstrate that the combinations of EZH2 inhibitors and IMiDs sensitized the cancer to the combination and improved the treatment effects of either compound alone. Thus, the combination may provide a useful treatment option for cancers resistant to a treatment, such as with a previous treatment with only one of the medications in the combination.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A method of treatment, comprising:
administering to a subject in need of a cancer treatment, a first compound comprising a compound of Formula I: 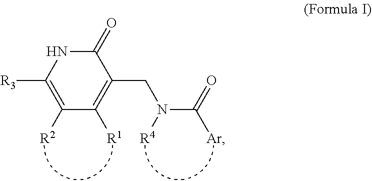
or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR⁵, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
administering to the subject a second compound comprising an immunomodulatory drug.

2. A composition for use in treating a subject in need of a cancer treatment, comprising:
a first compound comprising a compound of Formula I: 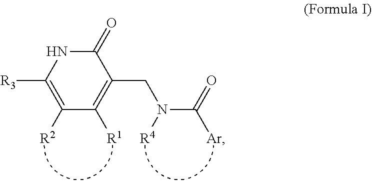
or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
a second compound comprising an immunomodulatory drug.

3. A combination comprising:
a first compound comprising a compound of Formula I: 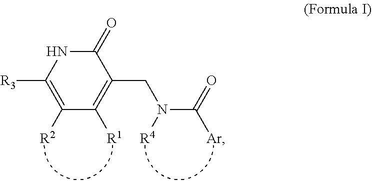
or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl;
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R, OR, SR, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring; and
a second compound comprising an immunomodulatory drug.

4. The method, composition, or combination of claim 1, 2 or 3, wherein the first compound is an Enhancer of Zeste Homolog 2 (EZH2) inhibitor.

5. The method, composition, or combination of claim 4, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.

6. The method, composition, or combination of any one of claims 1 to 5, wherein the immunomodulatory drug comprises an immunomodulatory imide drug (IMiD).

7. The method, composition, or combination of claim 6, wherein the IMiD comprises a cereblon modulator.

8. The method, composition, or combination of claim 7, wherein the cereblon modulator comprises a compound of Formula II: 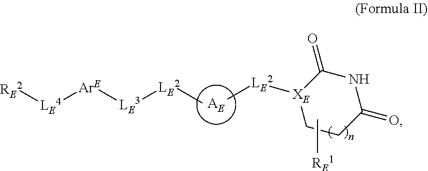
or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

9. The method, composition, or combination of claim 7 or 8, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.

10. The method, composition, or combination of claim 7 or 8, wherein the cereblon modulator excludes thalidomide, lenalidomide, and/or pomalidomide.

11. A method of treatment, comprising:
administering to a subject in need of a cancer treatment, a first compound comprising an EZH2 inhibitor; and
administering to the subject a second compound comprising a compound of Formula II: 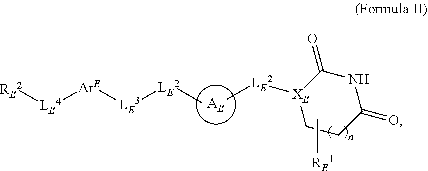
or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}?- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

12. A composition for use in treating a subject in need of a cancer treatment, comprising:
a first compound comprising an EZH2 inhibitor; and
a second compound comprising a compound of Formula II: 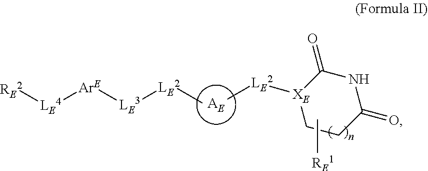
or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

13. A combination, comprising:
a first compound comprising an EZH2 inhibitor; and
a second compound comprising a compound of Formula II: 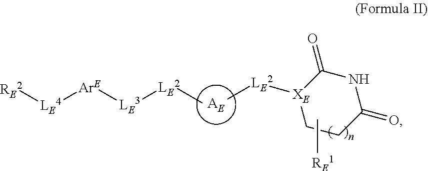
or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

14. The method, composition, or combination of any one of claims 11 to 13, wherein the EZH2 inhibitor comprises a compound of Formula I: 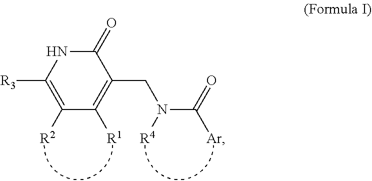
or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.

15. The method, composition, or combination of any one of claims 11 to 14, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.

16. The method, composition, or combination of any one of claims 11 to 15, wherein the compound of Formula II is a cereblon modulator.

17. The method, composition, or combination of claim 16, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.

18. The method, composition, or combination of claim 16, wherein the cereblon modulator excludes thalidomide, lenalidomide, and/or pomalidomide.

19. The method, composition, or combination of any one of claims 11 to 18, wherein the cancer is resistant to an EZH2 inhibitor or an IMiD.

20. A method of treatment, comprising:
identifying a subject having a relapsed or refractory cancer resistant to one or more EZH2 inhibitors or IMiDs;
administering to the subject a first compound comprising an EZH2 inhibitor; and
administering to the subject a second compound comprising an IMiD.

21. A composition for use in treating a subject identified as having a relapsed or refractory cancer resistant to one or more EZH2 inhibitors or IMiDs, comprising:
a first compound comprising an EZH2 inhibitor; and
a second compound comprising an IMiD.

22. A combination for use in treating relapsed or refractory cancer resistant to one or more EZH2 inhibitors or IMiDs, comprising:
a first compound comprising an EZH2 inhibitor; and
a second compound comprising an IMiD.

23. The method, composition, or combination of any one of claims 20 to 22, wherein the EZH2 inhibitor comprises a compound of Formula I: 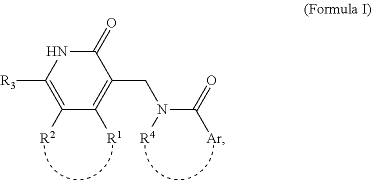
or a pharmaceutically acceptable salt thereof, wherein:
R¹ is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₁-C₈ alkylthio, optionally substituted C₃-C₁₂ cycloalkyl, optionally substituted C₃-C₁₂ cycloalkoxy, optionally substituted C₃-C₁₂ cycloalkylamino, optionally substituted C₃-C₁₂ cycloalkylthio, and optionally substituted 3-12 membered heterocyclyl; and
R² is selected from the group consisting of hydrogen, halogen, cyano, hydroxy, amino, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxy, optionally substituted C₁-C₈ alkylamino, optionally substituted C₃-C₁₂ cycloalkyl, optionally substituted C₃-C₁₂ cycloalkoxy, optionally substituted C₃-C₁₂ cycloalkylamino, and optionally substituted 3-12 membered heterocyclyl; or
R¹ and R², taken together with the atoms to which they are connected, can optionally form a 5-6 membered cycloalkyl, a 5-6 membered heterocyclyl, a 6 membered aryl, or a 5-6 membered heteroaryl;
R³ is selected from the group consisting of hydrogen, halogen, optionally substituted C₁-C₈ alkyl, optionally substituted C₁-C₈ heteroalkyl, optionally substituted C₃-C₁₂ cycloalkyl, optionally substituted 3-12 membered heterocyclyl, optionally substituted C₁-C₈ alkoxy, optionally substituted C₁-C₈ alkylamino, optionally substituted C₃-C₁₂ cycloalkoxy, and optionally substituted C₃-C₁₂ cycloalkylamino;
R⁴ is selected from the group consisting of hydrogen, optionally substituted C₁-C₈ alkyl, optionally substituted C₁-C₈ heteroalkyl, optionally substituted 3-12 membered cycloalkyl, and optionally substituted 3-12 membered heterocyclyl; or
R⁴ is a bond, optionally substituted C₁-C₈ alkylene, or optionally substituted C₁-C₈ heteroalkylene, and R⁴ connects to Ar, wherein R⁴ and Ar, together with the atoms to which they are connected, can optionally form a 4-7 membered heterocyclyl ring;
Ar is selected from aryl and heteroaryl, each of which could be mono-cyclic, bi-cyclic, or tri-cyclic rings that are optionally substituted with one or more substituents independently selected from the group consisting of R⁵, OR, SR⁵, NR⁶R⁷, S(O)R, S(O)₂R⁵, S(O)₂NR⁶R⁷, C(O)R⁵, and C(O)NR⁶R⁷;
R⁵, R⁶, and R⁷, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of each R⁵, R⁶ and/or R⁷, together with the atoms to which they are connected, can optionally form a 3-12 membered carbocyclyl ring, a 3-12 membered heterocyclyl ring, an aryl ring, or a heteroaryl ring.

24. The method, composition, or combination of any one of claims 20 to 23, wherein the EZH2 inhibitor is selected from the group consisting of: tazemetostat; lirametostat; N-((4,6-dimethyl-2-oxo-1,2-dihydropyridin-3-yl)methyl)-1-isopropyl-6-(6-(4-isopropylpiperazin-1-yl)pyridin-3-yl)-1H-indazole-4-carboxamide; (2R)-7-chloro-2-(4-(3-methoxyazetidin-1-yl)cyclohexyl)-2,4-dimethyl-N-((6-methyl-4-(methylthio)-2-oxo-1,2-dihydropyridin-3-yl)methyl)benzo[d]-[1,3]dioxole-5-carboxamide; (R)-9-chloro-2-((1r,4R)-4-(dimethylamino)cyclohexyl)-2,4-dimethyl-6-((6-methyl-4-(methylthio)-2-oxo-1,2-dihydropyridin-3-yl)methyl)-7,8-dihydro-[1,3]dioxolo[4,5-g]isoquinolin-5(6H)-one; (S)-5,8-dichloro-7-(methoxy(oxetan-3-yl)methyl)-2-((4-methoxy-6-methyl-2-oxo-1,2-dihydropyridin-3-yl)methyl)-3,4-dihydroisoquinolin-1(2H)-one; valemetostat; 1-isopropyl-6-(6-(4-isopropylpiperazin-1-yl)pyridin-3-yl)-N-((6-methyl-2-oxo-4-propyl-1,2-dihydropyridin-3-yl)methyl)-1H-indazole-4-carboxamide; N-((4,6-dimethyl-2-oxo-1,2-dihydropyridin-3-yl)methyl)-5-ethyl-6-(ethyl(tetrahydro-2H-pyran-4-yl)amino)-2-(piperidin-1-ylmethyl)benzofuran-4-carboxamide; and (S)-1-(sec-butyl)-N-((4,6-dimethyl-2-oxo-1,2-dihydropyridin-3-yl)methyl)-3-methyl-6-(6-(piperazin-1-yl)pyridin-3-yl)-1H-indole-4-carboxamide; or a pharmaceutically acceptable salt thereof.

25. The method, composition, or combination of any one of claims 20 to 23, wherein the IMiD comprises a cereblon modulator.

26. The method, composition, or combination of claim 25, wherein the cereblon modulator comprises a compound of Formula II: 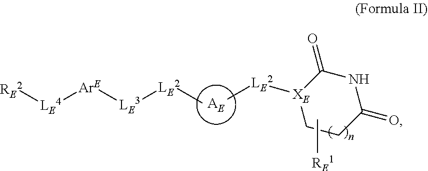
or a pharmaceutically acceptable salt thereof, wherein:
Ring A_{E} is a divalent group selected from 3-15 membered cycloalkyl, 3-15 membered heterocyclyl, 6-15 membered aryl, or 5-15 membered heteroaryl, each of which may be mono-cyclic, bi-cyclic, or tri-cyclic rings that are optionally substituted with one or more substituents independently selected from hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, optionally substituted 3-12 membered heterocyclyl, optionally substituted 6-membered aryl, and optionally substituted 5-6 membered heteroaryl;
X_{E} is selected from the group consisting of CR_{E}¹ and N;
R_{E}¹, at each occurrence, is selected from the group consisting of hydrogen, halogen, cyano, optionally substituted C₁-C₈ alkyl, optionally substituted C₁-C₈ heteroalkyl, optionally substituted C₃-C₁₂ cycloalkyl, and optionally substituted 3-12 membered heterocyclyl;
R_{E}² is selected from the group consisting of —R_{E}^{2b}-R_{E}^{2a};
wherein R_{E}^{2b} is null, or a divalent group selected from the group consisting of optionally substituted C₁-C₈ alkylene, optionally substituted C₂-C₈ alkenylene, optionally substituted C₂-C₈ alkynylene, optionally substituted C₁-C₈ heteroalkylene, optionally substituted C₂-C₈ heteroalkenylene, optionally substituted C₂-C₈ heteroalkynylene, optionally substituted C₃-C₁₂ membered cycloalkylene, optionally substituted 3-12 membered heterocyclylene, optionally substituted aryl, and optionally substituted heteroaryl;
R_{E}^{2a} is selected from the group consisting of R_{E}³, OR_{E}³, SR_{E}³, NR_{E}⁴R_{E}⁵, S(O)R_{E}³, S(O)₂R_{E}³, S(O)₂NR_{E}⁴R_{E}⁵, C(O)R_{E}³, and C(O)NR_{E}⁴R_{E}⁵;
R_{E}³, R_{E}⁴, and R_{E}⁵, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈ alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
R_{E}⁴ and R_{E}⁵, together with the atoms to which they are connected, can optionally form a C₃-C₁₂ membered cycloalkyl ring, or a 3-12 membered heterocyclyl ring;
L_{E}¹, L_{E}², L_{E}³ and L_{E}⁴ are divalent groups independently selected from the group consisting of -L_{E}^{a}-L_{E}^{b}-;
wherein L_{E}^{a} and L_{E}^{b}, at each occurrence, are independently selected from the group consisting of null, —CO—, —O—, —S—, —CR_{E}⁶R_{E}⁷- and —NR_{E}⁶-, with the proviso that -L_{E}^{a}-L_{E}^{b}- is not —O—O—;
R_{E}⁶ and R_{E}¹ are independently selected from the groups consisting of null, hydrogen, halogen, cyano, nitro, hydroxy, amino, optionally substituted C₁-C₈ alkyl, optionally substituted C₁-C₈ heteroalkyl, optionally substituted C₁-C₈ alkoxy, optionally substituted C₁-C₈ alkylamino, optionally substituted C₃-C₁₂ cycloalkyl, and optionally substituted 3-12 membered heterocyclyl; or
R_{E}⁶ and R_{E}⁷, together with the atoms to which they are connected, can optionally form a C₃-C₁₂ membered cycloalkyl ring, or a 3-12 membered heterocyclyl ring;
Ar_{E} is selected from aryl and heteroaryl, each of which is optionally substituted with one or more substituents independently selected from the group consisting of R_{E}⁸, OR_{E}⁸, SR_{E}⁸, NR_{E}⁹R_{E}¹⁰, S(O)R_{E}⁸, S(O)₂R_{E}⁸, S(O)₂NR_{E}⁹R_{E}¹⁰, C(O)R_{E}⁸, and C(O)NR_{E}⁹R_{E}¹⁰;
R_{E}⁸, R_{E}⁹, and R_{E}¹⁰, at each occurrence, are independently selected from the group consisting of null, hydrogen, halogen, cyano, nitro, optionally substituted C₁-C₈ alkyl, optionally substituted C₂-C₈ alkenyl, optionally substituted C₂-C₈ alkynyl, optionally substituted C₁-C₈ heteroalkyl, optionally substituted C₂-C₈ heteroalkenyl, optionally substituted C₂-C₈ heteroalkynyl, optionally substituted C₁-C₈ alkoxyC₁-C₈alkyl, optionally substituted C₁-C₈ haloalkyl, optionally substituted C₁-C₈ hydroxyalkyl, optionally substituted C₁-C₈alkylaminoC₁-C₈alkyl, optionally substituted C₃-C₁₂ cycloalkyl, optionally substituted 3-12 membered heterocyclyl, optionally substituted arylalkyl, optionally substituted heteroarylalkyl, optionally substituted aryl, and optionally substituted heteroaryl; or
two of independent R_{E}⁸, R_{E}⁹, and R_{E}¹⁰, together with the atoms to which they are connected, can optionally form a 3-12 membered carbocyclyl ring, a 3-12 membered heterocyclyl ring, an aryl ring, or an heteroaryl ring; and
n is selected from 0, 1, and 2.

27. The method, composition, or combination of claim 25 or 26, wherein the cereblon modulator is selected from the group consisting of: iberdomide; mezigdomide; (S)-2-(2,6-dioxopiperidin-3-yl)-4-((2-fluoro-4-((3-morpholinoazetidin-1-yl)methyl)benzyl)amino)isoindoline-1,3-dione; 3-(5-(1-benzylpiperidin-4-yl)-1-oxoisoindolin-2-yl)piperidine-2,6-dione; (S)-3-(6-(4-(morpholinomethyl)benzyl)-2-oxobenzo[cd]indol-1(2H)-yl)piperidine-2,6-dione; and avadomide; or a pharmaceutically acceptable salt thereof.

28. The method, composition, or combination of claim 25 or 26, wherein the cereblon modulator excludes thalidomide, lenalidomide, and/or pomalidomide.

29. The method, composition, or combination of any one of the aforementioned claims, wherein the first or second compound reduces cancer cell viability.

30. The method, composition, or combination of any one of the aforementioned claims, wherein the first and second compounds synergistically reduce cancer cell viability.

31. The method, composition, or combination of claim 29 or 30, wherein the cancer cell viability is reduced by at least 10%, at least 20%, or at least 50%.

32. The method, composition, or combination of any one of claims 29 to 31, wherein administration of the first and second compounds decreases the cancer cell viability in the subject.

33. The method, composition, or combination of any one of the aforementioned claims, wherein administration of the first and second compounds improves a cancer symptom in the subject, relative to a baseline cancer symptom.

34. The method, composition, or combination of claim 33, wherein the cancer symptom includes uncontrolled cell growth or division, unexplained pain, fever, night sweats, fatigue, unexplained weight loss, weakness, anorexia, or unexplained bleeding.

35. The method, composition, or combination of any one of the aforementioned claims, wherein the cancer comprises a blood cancer.

36. The method, composition, or combination of claim 35, wherein the blood cancer comprises a lymphoma, multiple myeloma, or leukemia.

37. The method, composition, or combination of claim 35, wherein the blood cancer comprises lymphoma.

38. The method, composition, or combination of claim 35 or 36, wherein the lymphoma comprises a non-Hodgkin's lymphoma.

39. The method, composition, or combination of claim 38, wherein the non-Hodgkin's lymphoma is follicular lymphoma (FL) or diffuse large B-cell lymphoma (DLBCL).

40. The method, composition, or combination of any one of the aforementioned claims, further comprising an additional cancer treatment to the subject.

41. The method, composition, or combination of any one of the aforementioned claims, wherein the additional cancer treatment comprises chemotherapy, immunotherapy, radiation therapy, stem cell transplantation, targeted therapy.

42. The method, composition, or combination of any one of the aforementioned claims, wherein the subject is a mammal.

43. The method, composition, or combination of any one of the aforementioned claims, wherein the subject is human.
